SEQUENCE LISTING (95 sequences)

SEQ ID NO: 1 - protein, 123 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCFDGFDIWGQGTMVTVSS

SEQ ID NO: 2 - protein, 8 aa, Homo sapiens
GFTFTSSA

SEQ ID NO: 3 - protein, 8 aa, Homo sapiens
IVVGSGNT

SEQ ID NO: 4 - protein, 18 aa, Homo sapiens
CAAPYCSGGSCFDGFDIW

SEQ ID NO: 5 - protein, 109 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGGLTGWTFGQGTKVEIR

SEQ ID NO: 6 - protein, 7 aa, Homo sapiens
QSVSSSY

SEQ ID NO: 7 - protein, 12 aa, synthetic construct
CQQYGGLTGWTF

SEQ ID NO: 8 - protein, 109 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYVGLTGWTFGQGTKVEIR

SEQ ID NO: 9 - protein, 12 aa, synthetic construct
CQQYVGLTGWTF

SEQ ID NO: 10 - protein, 123 aa, Homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCYDGFDIWGQGTMVTVSS

SEQ ID NO: 11 - protein, 18 aa, Homo sapiens
CAAPNCSNVVCYDGFDIW

SEQ ID NO: 12 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGGLTGWTFGQGTKVEIK

SEQ ID NO: 13 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYVGLTGWTFGQGTKVEIK

SEQ ID NO: 14 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYYCQQYVGLTGWTFGQGTKVEIK

SEQ ID NO: 15 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCADGFDIWGQGTMVTVSS

SEQ ID NO: 16 - protein, 18 aa, synthetic construct
CAAPNCSNVVCADGFDIW

SEQ ID NO: 17 - protein, 109 aa, synthetic construct
DIVLTQTPGTLSLSPGERATLSCRASQSVSSSYLAWYQQKPGQAPRLLIYGASSRATGIPDRFSGSGSGTDFTLTISRLEPEDFAVYYCQQYVGLTGWTFGQGTKVEIK

SEQ ID NO: 18 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSNVVCFDGFDIWGQGTMVTVSS

SEQ ID NO: 19 - protein, 18 aa, synthetic construct
CAAPYCSNVVCFDGFDIW

SEQ ID NO: 20 - protein, 108 aa, Homo sapiens
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPWTFGQGTKVEIR

SEQ ID NO: 21 - protein, 11 aa, Homo sapiens [sequence omitted]

SEQ ID NO: 22 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGVVCFDGFDIWGQGTMVTVSS

SEQ ID NO: 23 - protein, 18 aa, synthetic construct
CAAPYCSGVVCFDGFDIW

SEQ ID NO: 24 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGVCFDGFDIWGQGTMVTVSS

SEQ ID NO: 25 - protein, 18 aa, synthetic construct
CAAPYCSGGVCFDGFDIW

SEQ ID NO: 26 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCYDGFDIWGQGTMVTVSS

SEQ ID NO: 27 - protein, 18 aa, synthetic construct
CAAPYCSGGSCYDGFDIW

SEQ ID NO: 28 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSGGSCFDGFDIWGQGTMVTVSS

SEQ ID NO: 29 - protein, 18 aa, synthetic construct
CAAPNCSGGSCFDGFDIW

SEQ ID NO: 30 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSNGVCFDGFDIWGQGTMVTVSS

SEQ ID NO: 31 - protein, 18 aa, synthetic construct
CAAPYCSNGVCFDGFDIW

SEQ ID NO: 32 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSGGVCYDGFDIWGQGTMVTVSS

SEQ ID NO: 33 - protein, 18 aa, synthetic construct
CAAPNCSGGVCYDGFDIW

SEQ ID NO: 34 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSGGVCFDGFDIWGQGTMVTVSS

SEQ ID NO: 35 - protein, 18 aa, synthetic construct
CAAPNCSGGVCFDGFDIW

SEQ ID NO: 36 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSNVSCFDGFDIWGQGTMVTVSS

SEQ ID NO: 37 - protein, 17 aa, synthetic construct
AAPYCSNVSCFDGFDIW

SEQ ID NO: 38 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVSCFDGFDIWGQGTMVTVSS

SEQ ID NO: 39 - protein, 18 aa, synthetic construct
CAAPNCSNVSCFDGFDIW

SEQ ID NO: 40 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGVVCYDGFDIWGQGTMVTVSS

SEQ ID NO: 41 - protein, 18 aa, synthetic construct
CAAPYCSGVVCYDGFDIW

SEQ ID NO: 42 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGVSCFDGFDIWGQGTMVTVSS

SEQ ID NO: 43 - protein, 18 aa, synthetic construct
CAAPYCSGVSCFDGFDIW

SEQ ID NO: 44 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSNGSCFDGFDIWGQGTMVTVSS

SEQ ID NO: 45 - protein, 18 aa, synthetic construct
CAAPYCSNGSCFDGFDIW

SEQ ID NO: 46 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCSDGFDIWGQGTMVTVSS

SEQ ID NO: 47 - protein, 18 aa, synthetic construct
CAAPYCSGGSCSDGFDIW

SEQ ID NO: 48 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPYCSGGSCGDGFDIWGQGTMVTVSS

SEQ ID NO: 49 - protein, 18 aa, synthetic construct
CAAPYCSGGSCGDGFDIW

SEQ ID NO: 50 - protein, 108 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYVNSPWTFGQGTKVEIR

SEQ ID NO: 51 - protein, 11 aa, synthetic construct [sequence omitted]

SEQ ID NO: 52 - protein, 108 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGGSPWTFGQGTKVEIR

SEQ ID NO: 53 - protein, 11 aa, synthetic construct [sequence omitted]

SEQ ID NO: 54 - protein, 108 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGNLPWTFGQGTKVEIR

SEQ ID NO: 55 - protein, 11 aa, synthetic construct [sequence omitted]

SEQ ID NO: 56 - protein, 108 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSTWTFGQGTKVEIR

SEQ ID NO: 57 - protein, 11 aa, synthetic construct [sequence omitted]

SEQ ID NO: 58 - protein, 108 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGGLTWTFGQGTKVEIR

SEQ ID NO: 59 - protein, 11 aa, synthetic construct
CQQYGGLTWTF

SEQ ID NO: 60 - protein, 108 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYVGLTWTFGQGTKVEIR

SEQ ID NO: 61 - protein, 11 aa, synthetic construct
CQQYVGLTWTF

SEQ ID NO: 62 - protein, 109 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGNLTGWTFGQGTKVEIR

SEQ ID NO: 63 - protein, 12 aa, synthetic construct
CQQYGNLTGWTF

SEQ ID NO: 64 - protein, 109 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSTGWTFGQGTKVEIR

SEQ ID NO: 65 - protein, 12 aa, synthetic construct
CQQYGNSTGWTF

SEQ ID NO: 66 - protein, 109 aa, synthetic construct
EIVLTQSPGTLSLSPGERATLSCRASQSVSSSYLAWYQQKPGQAPRLLIYGASSRATGFPDRFSGSGSGTDFTLTISRLEPEDFAVYYCQQYGNSPGWTFGQGTKVEIR

SEQ ID NO: 67 - protein, 12 aa, synthetic construct
CQQYGNSPGWTF

SEQ ID NO: 68 - protein, 123 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAPNCSNVVCSDGFDIWGQGTMVTVSS

SEQ ID NO: 69 - protein, 18 aa, synthetic construct
CAAPNCSNVVCSDGFDIW

SEQ ID NO: 70 - protein, 108 aa, Homo sapiens
EIVLTQSPGTLSLSPGERATLSCRASQSVSSSYLAWYQQKPGQAPRLLIYSASSRATGIPDRFSGSGSGTDFTLTISRLEPEDFAVYFCQQYGTSPWTFGQGTKVEIK

SEQ ID NO: 71 - protein, 11 aa, Homo sapiens [sequence omitted]

SEQ ID NO: 72 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYFCQQYGTSPWTFGQGTKVEIK

SEQ ID NO: 73 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYYCQQYGTSPWTFGQGTKVEIK

SEQ ID NO: 74 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYVTSPWTFGQGTKVEIK

SEQ ID NO: 75 - protein, 11 aa, synthetic construct [sequence omitted]

SEQ ID NO: 76 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGGSPWTFGQGTKVEIK

SEQ ID NO: 77 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGTLPWTFGQGTKVEIK

SEQ ID NO: 78 - protein, 11 aa, synthetic construct [sequence omitted]

SEQ ID NO: 79 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGTSTWTFGQGTKVEIK

SEQ ID NO: 80 - protein, 11 aa, synthetic construct [sequence omitted]

SEQ ID NO: 81 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGGLTWTFGQGTKVEIK

SEQ ID NO: 82 - protein, 108 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYVGLTWTFGQGTKVEIK

SEQ ID NO: 83 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGTLTGWTFGQGTKVEIK

SEQ ID NO: 84 - protein, 12 aa, synthetic construct
CQQYGTLTGWTF

SEQ ID NO: 85 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGTSTGWTFGQGTKVEIK

SEQ ID NO: 86 - protein, 12 aa, synthetic construct
CQQYGTSTGWTF

SEQ ID NO: 87 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYFCQQYGTSPGWTFGQGTKVEIK

SEQ ID NO: 88 - protein, 12 aa, synthetic construct [sequence omitted]

SEQ ID NO: 89 - protein, 109 aa, synthetic construct
EIVLTQSPGTLSLSPGERATLSCRASQSVSSSYLAWYQQKPGQAPRLLIYSASSRATGIPDRFSGSGSGTDFTLTISRLEPEDFAVYYCQQYGGLTGWTFGQGTKVEIK

SEQ ID NO: 90 - protein, 123 aa, synthetic construct
QVQLVQSGPEVKKPGTSVRVSCKASGFTFTSSAVQWVRQARGQRLEWVGWIVVGSGNTNYAQKFHERVTITRDMSTSTAYMELSSLRSEDTAVYYCASPYCSGGSCSDGFDIWGQGTMVTVSS

SEQ ID NO: 91 - protein, 7 aa, synthetic construct
IVVGSGN

SEQ ID NO: 92 - protein, 18 aa, synthetic construct
CASPYCSGGSCSDGFDIW

SEQ ID NO: 93 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGNSPWTFGQGTKVEIR

SEQ ID NO: 94 - protein, 1270 aa, Severe acute respiratory syndrome-related coronavirus
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHENNKSRMESELRVYSSANNCTFEYVSQPFLMDLEGKQGNFKNLREFVFKNIDGYFKIYSKHTPVNLGRDLPQGFSALEPLVDLPIGINITRFQTLLALHRSYLTPGDSSSSWTAGAAAYYVGYLQPRTFLLKYNENGTITDAVDCALDPLSETKCTLKSFTVEKGIYQTSNFRVQPTESIVRFPNITNLCPFHEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 95 - protein, 1268 aa, Severe acute respiratory syndrome-related coronavirus
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2026/0146077 A1
(43) Pub. Date: May 28, 2026

(54) ENGINEERED SARS-COV-2 ANTIBODIES WITH INCREASED NEUTRALIZATION BREADTH

(71) Applicant: The U.S.A., as represented by the Secretary, Department of Health and Human Services, Bethesda, MD (US)

(72) Inventors: John Nicholas Misasi, Dedham, MD (US); Lingshu Wang, North Potomac, MD (US); Nancy J. Sullivan, Brookline, MD (US); John R. Mascola, Rockville, MD (US); Misook Choe, Potomac, MD (US); Tongqing Zhou, Boyds, MD (US); Peter D. Kwong, Washington, DC (US); Richard Alan Koup, Washington, DC (US); Wei Shi, Rockville, MD (US); Eun Sung Yang, Rockville, MD (US); Yi Zhang, Maryland, MD (US); Man Chen, Clarksburg, MD (US)

(73) Assignee: The U.S.A., as represented by the Secretary, Department of Health and Human Services, Bethesda, MD (US)

(21) Appl. No.: 19/109,315

(22) PCT Filed: Sep. 6, 2023
(86) PCT No.: PCT/US2023/073517

§ 371 Date: Mar. 6, 2025

Related U.S. Application Data
(60) Provisional application No. 63/404,473, filed on Sep. 7, 2022.

(51) Int. Cl. C07K 16/104; A61K 31/7088; A61K 38/00; C07K 1/14; C12N 15/11; C12N 15/63; G01N 33/569; G01N 33/577

(52) U.S. Cl. CPC C07K 16/104; A61K 31/7088; A61K 38/00; C07K 1/14; C12N 15/11; C12N 15/63; G01N 33/56983; G01N 33/577; C07K 2317/21; C07K 2317/31; C07K 2317/52; C07K 2317/54; C07K 2317/55; C07K 2317/565; C07K 2317/567; C07K 2317/622; C07K 2317/72; C07K 2317/76; C07K 2317/94; C12N 2830/34

(57) ABSTRACT

Disclosed are monoclonal antibodies, antigen binding fragments, and bi-specific antibodies that specifically bind SARS-CoV-2. Also disclosed is the use of these antibodies for inhibiting a coronavirus infection, such as a SARS-CoV-2 infection. In addition, disclosed are methods for detecting a coronavirus, such as SARS-CoV-2, in a biological sample, using the disclosed antibodies. In some embodiments, the SARS-CoV-2 is the BA.4 or BA.5 variant.

### CROSS REFERENCE TO RELATED APPLICATION(S)

This claims the benefit of U.S. Provisional Application No. 63/404,473, filed Sep. 7, 2022, which is incorporated herein by reference.

### FIELD OF THE DISCLOSURE

This relates to monoclonal antibodies and antigen binding fragments that specifically bind a severe acute respiratory syndrome coronavirus 2 (SARS-CoV-2) spike protein, and their use for inhibiting SARS-CoV-2 infection in a subject, and their use for detecting SARS-CoV-2.

### REFERENCE TO AN ELECTRONIC SEQUENCE LISTING

The contents of the electronic sequence listing, entitled sequences.xml, which has a size of 91,258 bytes and a Date of Creation of Aug. 3, 2023, is herein incorporated by reference in its entirety.

### BACKGROUND

In 2019, the International Committee on the Taxonomy of Viruses (ICTV) describes the Coronaviridae subfamily Orthocoronavirinae which included several viruses that are pathogenic to humans. The most common human coronaviruses cause the common cold and include the alpha-coronaviruses 229E and NL63, and the beta-coronaviruses OC43 and HKU1. In addition to the coronaviruses that cause common cold symptoms, three beta-coronaviruses have been shown to be highly pathogenic in humans. These viruses, Middle East Respiratory Syndrome Coronavirus (MERS), Severe Acute Respiratory Syndrome Coronavirus 1 (SARS-CoV-1) and SARS-CoV-2, can produce severe symptoms that can lead to death in human patients.

The genome of coronavirus is a large, enveloped, positive-sense, single-stranded RNA whose genome length varies by species and encodes multiple structural and non-structural proteins, encoded in several reading frames. The Spike protein(S) is expressed on the surface of the viral particle and is responsible for virus entry and infection of target cells. Transmission of coronaviruses can occur through multiple methods, including respiratory droplets, aerosols, fecal-oral and fomite routes.

At the end of 2019, a novel coronavirus was identified as the cause of a serve respiratory distress syndrome outbreak. This virus was later sequenced and identified to be highly similar to SARS-CoV-1 and based on this result, the novel Coronavirus was renamed SARS-CoV-2. The incubation period is typically between 4 to 14 days but can be as short as 1 day. Infection is characterized by fever, fatigue, cough, difficulty breathing and diarrhea. A subset of patients has significant respiratory distress, requiring hospitalization and oxygen supplementation. These patients can rapidly deteriorate and require intensive care unit admission and intubation. Severe disease is also characterized by abnormalities in multi-organ failure, blood clots and an apparent systemic inflammatory response syndrome.

The Omicron (Pango lineage B.1.1.529) initially emerged as the BA. 1 sub-lineage. BA.1 was first detected by genomic surveillance in South Africa and showed extensive immune escape. The BA.4 and BA.5 sub-lineages, which do not differ in their spike sequence from each other, were also first detected by genomic surveillance in South Africa. BA.4 and BA.5 have changes relative to the BA. 1 and BA.2 sub-lineages including the L452R and F486V mutations and the R493Q reversion in the spike receptor binding domain (RBD). BA.4 and BA.5 also differ from the BA.2 sub-lineage by a deletion of spike residues 69 and 70 (Khan et al., Nature Comm. 13. Article number 4686, doi.org/10.1038/s41467-022-32396-9, (2022)).

A need remains for antibodies that are highly potent for binding SARS-CoV-2, and can be used as therapeutics and diagnostics, such as to detect Omicron and the BA.4 and BA.5 variants.

### SUMMARY

Monoclonal antibodies that specifically bind SARS-CoV-2, are disclosed herein. In some embodiments, the antibodies bind a BA.4 or BA.5 variant. The antibodies are potent neutralizing antibodies and target unique epitopes in the spike glycoprotein of SARS-CoV-2.

In some embodiments, the antibody specifically binds to a SARS-CoV-2 spike protein and incudes a heavy chain variable (V_{H}) region and a light chain variable region (V_{L}) including a heavy chain complementarity determining region (HCDR)1, a HCDR2, and a HCDR3, and a light chain complementarity determining region (LCDR)1, a LCDR2, and a LCDR3 of the V_{H} and V_{L} set forth as one of:
a) SEQ ID NOs: 1 and 5, respectively (B1-182.1HC/182.1LC_94GLTG);
b) SEQ ID NOs: 1 and 8, respectively (B1-182.1HC/182.1LC_93VGLTG);
c) SEQ ID NOs: 10 and 12, respectively (A23-58.1HC/58.1LC_94GLTG);
d) a V_{H} and a V_{L} including a HCDR1, a HCDR2, and a HCDR3, and a LCDR1, a LCDR2, and a LCDR3 of the V_{H} and V_{L} set forth as SEQ ID NOs: 10 and 13, respectively (A23-58.1HC/58.1LC_93VGLTG);
e) SEQ ID NOs: 10 and 14, respectively (A23-58.1HC/58.1LC_F88Y-93VGLTG);
f) SEQ ID NOs: 15 and 13, respectively (A23-58.1HC_Y100cA/58.1LC_93VGLTG);
g) SEQ ID NOs: 10 and 17, respectively (A23-58.1HC/S2E12LC);
h) SEQ ID NOs: 15 and 17, respectively (A23-58.1HC_Y100cA/S2E12LC);
i) SEQ ID NOs: 1 and 17, respectively (B1-182.1HC/S2E12LC);
j) SEQ ID NOs: 18 and 20, respectively (B1-182.1HC_GGStoNVV/182.1LC);
k) SEQ ID NOs: 22 and 20, respectively (B1-182.1HC_GStoVV/182.1LC);
l) SEQ ID NOs: 24 and 20, respectively (B1-182.1HC_S100aV/182.1LC);
m) SEQ ID NOs: 26 and 20, respectively (B1-182.1HC_F100cY/182.ILC);
n) SEQ ID NOs: 28 and 20, respectively (B1-182.1HC_Y96N/182.1LC);
o) SEQ ID NOs: 30 and 20, respectively (B1-182.1HC_G-StoN-V/182.1LC);
p) SEQ ID NOs: 32 and 20, respectively (B1-182.1HC_YSFtoNVY/182.1LC);
q) SEQ ID NOs: 34 and 20, respectively (B1-182.1HC_Y-StoN-V/182.1LC);
r) SEQ ID NOs: 36 and 20, respectively (B1-182.1HC_GGtoNV/182.1LC);
s) SEQ ID NOs: 38 and 20, respectively (B1-182.1HC_YGGtoNNV/182.1LC);
t) SEQ ID NOs: 40 and 20, respectively (B1-182.1HC_-GSFto-VVY/182.ILC);
u) SEQ ID NOs: 42 and 20, respectively (B1-182.1HC_G100V/182.1LC);
v) SEQ ID NOs: 44 and 20, respectively (B1-182.1HC_G99N/182.1LC);
w) SEQ ID NOs: 46 and 20, respectively (B1-182.1HC_F100cS/182.1LC);
x) SEQ ID NOs: 1 and 50, respectively (B1-182.1HC/182.1LC_G93V);
y) SEQ ID NOs: 1 and 52, respectively (B1-182.1HC/182.1LC_N94G);
z) SEQ ID NOs: 1 and 54, respectively (B1-182.1HC/182.1LC_S95L);
aa) SEQ ID NOs: 1 and 58, respectively (B1-182.1HC/182.1LC_94GLT);
bb) SEQ ID NOs: 1 and 62, respectively (B1-182.1HC/182.1LC_95LTG);
cc) SEQ ID NOs: 1 and 64, respectively (B1-182.1HC/182.1LC_96TG);
dd) SEQ ID NOs: 1 and 66, respectively (B1-182.1HC/182.1LC_96+G);
ee) SEQ ID NOs: 46 and 5, respectively (B1-182.1HC_F100S/182.1LC_94GLTG);
ff) SEQ ID NOs: 46 and 8, respectively (B1-182.1HC_F100cS/182.1LC_93VGLTG);
gg) SEQ ID NOs: 68 and 70, respectively (A23-58.1HC_Y100cS/58.1LC);
hh) SEQ ID NOs: 15 and 70, respectively (A23-58.1HC_Y100cA/58.1LC);
ii) SEQ ID NOs: 10 and 72, respectively (A23-58.1HC/58.1LC_S51G);
jj) SEQ ID NOs: 10 and 73, respectively (A23-58.1HC/58.1LC_F88Y);
kk) SEQ ID NOs: 10 and 74, respectively (A23-58.1HC/58.1LC_G93V);
ll) SEQ ID NOs: 10 and 76, respectively (A23-58.1HC/58.1LC_T94G);
mm) SEQ ID NOs: 10 and 77, respectively (A23-58.1HC/58.1LC_S95L);
nn) SEQ ID NOs: 10 and 79, respectively (A23-58.1HC/58.1LC_P96T);
oo) SEQ ID NOs: 10 and 81, respectively (A23-58.1HC/58.1LC_94GLT);
pp) SEQ ID NOs: 10 and 82, respectively (A23-58.1HC/58.1LC_93VGLT);
qq) SEQ ID NOs: 10 and 83, respectively (A23-58.1HC/58.1LC_95LTG); IT) SEQ ID NOs: 10 and 85, respectively (A23-58.1HC/58.1LC_96TG);
ss) SEQ ID NOs: 10 and 89, respectively (A23-58.1HC/58.1LC_F88Y-94GLTG);
tt) SEQ ID NOs: 15 and 77, respectively (A23-58.1HC_Y100cA/58.1LC_S95L);
uu) SEQ ID NOs: 15 and 12, respectively (A23-58.1HC_Y100cA/58.1LC_94GLTG); or
vv) SEQ ID NOs: 46 and 17, respectively (B1-182.1HC_F100cS/S2E12LC).

In some embodiments, antigen binding fragments of these monoclonal antibodies are provided.

In further embodiments, disclosed are nucleic acid molecules encoding the V_{H} and/or V_{L} of the antibody, or antigen binding fragment, vectors including these nucleic acid molecules, and host cells transformed with these vectors.

In some embodiments, multi-specific antibodies including the disclosed monoclonal antibodies and antigen binding fragments. Methods of producing the disclosed monoclonal antibodies and antigen binding fragments are also disclosed.

In some embodiments, these antibodies are of use for inhibiting a SARS-CoV-2 infection, and for detecting SARS-CoV-2 in a biological sample.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1F. Neutralization activity of evaluated antibodies. Single cycle lentivirus particles pseudotyped with the indicated SARS-CoV-2 spike proteins were incubated with serial dilutions of the indicated antibodies and then added to pre-seeded 293T-ACE2 cells. Infection was determined by measuring luciferase signal and calculating the inhibitory concentration of antibody the reduces the signal by 50% (IC50) or 80% (IC80). n.t.=not tested. The sequence of BA.2.75 is provided as SEQ ID NO: 94. The sequence of BA.4.6 is provided as SEQ ID NO: 95.

### SEQUENCES

The nucleic and amino acid sequences are shown using standard letter abbreviations for nucleotide bases, and one letter code for amino acids, as defined in 37 C.F.R. 1.822. Only one strand of each nucleic acid sequence is shown, but the complementary strand is understood as included by any reference to the displayed strand. The sequences are also provided in Table A below.

TABLE A
Amino acid sequences and their corresponding SEQ ID NO.

| SEQ | |
|---|---|
| ID | |
| NO: | Sequence |
| 1 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGGSCFDGEDIWGQGTMVTVSS |
| 2 | GFTFTSSA |
| 3 | IVVGSGNT |
| 4 | CAAPYCSGGSCFDGFDIW |
| 5 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGGLTGWTFGQGTKVEIR |
| 6 | QSVSSSY |
| 7 | CQQYGGLTGWTF |
| 8 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYVGLTGWTFGQGTKVEIR |
| 9 | CQQYVGLTGWTF |
| 10 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSNVVCYDGFDIWGQGTMVTVSS |
| 11 | CAAPNCSNVVCYDGFDIW |
| 12 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGGLTGWTFGQGTKVEIK |
| 13 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYVGLTGWTFGQGTKVEIK |
| 14 | EIVLTQSPGTLSLSPGERATLSCRASQSVSSSYLAWYQQKPGQAPRLLIYSASSRATGIPDRFSGSGSG |
| | TDFTLTISRLEPEDFAVYYCQQYVGLTGWTFGQGTKVEIK |
| 15 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSNVVCADGFDIWGQGTMVTVSS |
| 16 | CAAPNCSNVVCADGFDIW |
| 17 | DIVLTQTPGTLSLSPGERATLSCRASQSVSSSYLAWYQQKPGQAPRLLIYGASSRATGIPDRFSGSGSG |
| | TDFTLTISRLEPEDFAVYYCQQYVGLIGWTFGQGTKVEIK |
| 18 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSNVVCFDGFDIWGQGTMVTVSS |
| 19 | CAAPYCSNVVCFDGFDIW |
| 20 | EIVLTQSPGTLSLSPGERATLSCRASQSVSSSYLAWYQQKPGQAPRLLIYGASSRATGFPDRESGSGSG |
| | TDFTLTISRLEPEDFAVYYCQQYGNSPWTFGQGTKVEIR |
| 21 | CQQYGNSPWTF |
| 22 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGVVCFDGFDIWGQGTMVTVSS |
| 23 | CAAPYCSGVVCFDGFDIW |
| 24 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGGVCFDGFDIWGQGTMVTVSS |
| 25 | CAAPYCSGGVCFDGFDIW |
| 26 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGGSCYDGFDIWGQGTMVTVSS |
| 27 | CAAPYCSGGSCYDGFDIW |
| 28 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSGGSCFDGEDIWGQGTMVTVSS |
| 29 | CAAPNCSGGSCFDGFDIW |
| 30 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSNGVCFDGFDIWGQGTMVTVSS |
| 31 | CAAPYCSNGVCFDGFDIW |
| 32 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSGGVCYDGFDIWGQGTMVTVSS |
| 33 | CAAPNCSGGVCYDGFDIW |
| 34 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSGGVCFDGFDIWGQGTMVTVSS |
| 35 | CAAPNCSGGVCFDGFDIW |
| 36 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSNVSCFDGFDIWGQGTMVTVSS |
| 37 | AAPYCSNVSCFDGFDIW |
| 38 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSNVSCFDGFDIWGQGTMVTVSS |
| 39 | CAAPNCSNVSCFDGFDIW |
| 40 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGVVCYDGFDIWGQGTMVTVSS |
| 41 | CAAPYCSGVVCYDGFDIW |
| 42 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGVSCFDGFDIWGQGTMVTVSS |
| 43 | CAAPYCSGVSCFDGFDIW |
| 44 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSNGSCFDGFDIWGQGTMVTVSS |
| 45 | CAAPYCSNGSCFDGFDIW |
| 46 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGGSCSDGFDIWGQGTMVTVSS |
| 47 | CAAPYCSGGSCSDGFDIW |
| 48 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPYCSGGSCGDGFDIWGQGTMVTVSS |
| 49 | CAAPYCSGGSCGDGFDIW |
| 50 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYVNSPWTFGQGTKVEIR |
| 51 | CQQYVNSPWTF |
| 52 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGGSPWTFGQGTKVEIR |
| 53 | CQQYGGSPWTF |
| 54 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGNLPWTFGQGTKVEIR |
| 55 | CQQYGNLPWTF |
| 56 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGNSTWTFGQGTKVEIR |
| 57 | CQQYGNSTWTF |
| 58 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGGLTWTFGQGTKVEIR |
| 59 | CQQYGGLTWTF |
| 60 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYVGLTWTFGQGTKVEIR |
| 61 | CQQYVGLTWTF |
| 62 | EIVLTQSPGTLSLSPGERATLSCRASQSVSSSYLAWYQQKPGQAPRLLIYGASSRATGFPDRESGSGSG |
| | TDFTLTISRLEPEDFAVYYCQQYGNLTGWTFGQGTKVEIR |
| 63 | CQQYGNLTGWTF |
| 64 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGNSTGWTFGQGTKVEIR |
| 65 | CQQYGNSTGWTF |
| 66 | EIVLTQSPGTLSLSPGERATLSCRASQSVSSSYLAWYQQKPGQAPRLLIYGASSRATGFPDRFSGSGSG |
| | TDFTLTISRLEPEDFAVYYCQQYGNSPGWTFGQGTKVEIR |
| 67 | CQQYGNSPGWTF |
| 68 | QMQLVQSGPEVKKPGTSVKVSCKASGFTFTSSAVQWVRQARGQRLEWIGWIVVGSGNTNYAQKFQERVT |
| | ITRDMSTSTAYMELSSLRSEDTAVYYCAAPNCSNVVCSDGFDIWGQGTMVTVSS |
| 69 | CAAPNCSNVVCSDGFDIW |
| 70 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGTSPWTFGQGTKVEIK |
| 71 | CQQYGTSPWTF |
| 72 | EIVLTQSPGTLSLSPGERATLSCRASQSVSSSYLAWYQQKPGQAPRLLIYGASSRATGIPDRFSGSGSG |
| | TDFTLTISRLEPEDFAVYFCQQYGTSPWTFGQGTKVEIK |
| 73 | EIVLTQSPGTLSLSPGERATLSCRASQSVSSSYLAWYQQKPGQAPRLLIYSASSRATGIPDRFSGSGSG |
| | TDFTLTISRLEPEDFAVYYCQQYGTSPWTFGQGTKVEIK |
| 74 | EIVLTQSPGTLSLSPGERATLSCRASQSVSSSYLAWYQQKPGQAPRLLIYSASSRATGIPDRESGSGSG |
| | TDFTLTISRLEPEDFAVYFCQQYVTSPWTFGQGTKVEIK |
| 75 | CQQYVTSPWTF |
| 76 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGGSPWTFGQGTKVEIK |
| 77 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGTLPWTFGQGTKVEIK |
| 78 | CQQYGTLPWTF |
| 79 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGTSTWTFGQGTKVEIK |
| 80 | CQQYGTSTWTF |
| 81 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGGLTWTFGQGTKVEIK |
| 82 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYVGLTWTFGQGTKVEIK |
| 83 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGTLTGWTFGQGTKVEIK |
| 84 | CQQYGTLTGWTF |
| 85 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGTSTGWTFGQGTKVEIK |
| 86 | CQQYGTSTGWTF |
| 87 | EIVLTQSPGTLSLSPGERATLSCRASQSVSSSYLAWYQQKPGQAPRLLIYSASSRATGIPDRFSGSGSG |
| | TDFTLTISRLEPEDFAVYFCQQYGTSPGWTFGQGTKVEIK |
| 88 | CQQYGTSPGWTF |
| 89 | EIVLTQSPGTLSLSPGERATLSCRASQSVSSSYLAWYQQKPGQAPRLLIYSASSRATGIPDRFSGSGSG |
| | TDFTLTISRLEPEDFAVYYCQQYGGLTGWTFGQGTKVEIK |
| 90 | QVQLVQSGPEVKKPGTSVRVSCKASGFTFTSSAVQWVRQARGQRLEWVGWIVVGSGNTNYAQKFHERVT |
| | ITRDMSTSTAYMELSSLRSEDTAVYYCASPYCSGGSCSDGFDIWGQGTMVTVSS |
| 91 | IVVGSGN |
| 92 | CASPYCSGGSCSDGFDIW |
| 93 | EIVLTQSPGTLSLSPGERATLSCRASQSVSSSYLAWYQQKPGQAPRLLIYGASSRATGIPDRFSGSGSG |
| | TDFTLTISRLEPEDFAVYYCQQYGNSPWTFGQGTKVEIR |
| 94 | MFVFLVLLPLVSSQCVNLITRTQSYTNSFTRGVYYPDKVFRSSVLHSTQDLFLPFFSNVTWFHAIHVSG |
| | TNGTKRFDNPVLPFNDGVYFASTEKSNIIRGWIFGTTLDSKTQSLLIVNNATNVVIKVCEFQFCNDPFL |
| | DVYYHENNKSRMESELRVYSSANNCTFEYVSQPFLMDLEGKQGNFKNLREFVFKNIDGYFKIYSKHTPV |
| | NLGRDLPQGFSALEPLVDLPIGINITRFQTLLALHRSYLTPGDSSSSWTAGAAAYYVGYLQPRTFLLKY |
| | NENGTITDAVDCALDPLSETKCTLKSFTVEKGIYQTSNFRVQPTESIVRFPNITNLCPFHEVENATRFA |
| | SVYAWNRKRISNCVADYSVLYNFAPFFAFKCYGVSPTKLNDLCFTNVYADSFVIRGNEVSQIAPGQTGN |
| | IADYNYKLPDDFTGCVIAWNSNKLDSKVSGNYNYLYRLFRKSKLKPFERDISTEIYQAGNKPCNGVAGF |
| | NCYFPLQSYGFRPTYGVGHQPYRVVVLSFELLHAPATVCGPKKSTNLVKNKCVNFNFNGLTGTGVLTES |
| | NKKFLPFQQFGRDIADTTDAVRDPQTLEILDITPCSFGGVSVITPGTNTSNQVAVLYQGVNCTEVPVAI |
| | HADQLTPTWRVYSTGSNVFQTRAGCLIGAEYVNNSYECDIPIGAGICASYQTQTKSHRRARSVASQSII |
| | AYTMSLGAENSVAYSNNSIAIPTNFTISVTTEILPVSMTKTSVDCTMYICGDSTECSNLLLQYGSFCTQ |
| | LKRALTGIAVEQDKNTQEVFAQVKQIYKTPPIKYFGGFNFSQILPDPSKPSKRSFIEDLLENKVTLADA |
| | GFIKQYGDCLGDIAARDLICAQKFNGLTVLPPLLTDEMIAQYTSALLAGTITSGWTFGAGAALQIPFAM |
| | QMAYRENGIGVTQNVLYENQKLIANQFNSAIGKIQDSLSSTASALGKLQDVVNHNAQALNTLVKQLSSK |
| | FGAISSVLNDILSRLDKVEAEVQIDRLITGRLQSLQTYVTQQLIRAAEIRASANLAATKMSECVLGQSK |
| | RVDFCGKGYHLMSFPQSAPHGVVFLHVTYVPAQEKNFTTAPAICHDGKAHFPREGVFVSNGTHWFVTQR |
| | NFYEPQIITTDNTFVSGNCDVVIGIVNNTVYDPLQPELDSFKEELDKYFKNHTSPDVDLGDISGINASV |
| | VNIQKEIDRLNEVAKNLNESLIDLQELGKYEQYIKWPWYIWLGFIAGLIAIVMVTIMLCCMTSCCSCLK |
| | GCCSCGSCCKFDEDDSEPVLKGVKLHYT |
| 95 | MFVFLVLLPLVSSQCVNLITRTQSYTNSFTRGVYYPDKVFRSSVLHSTQDLFLPFFSNVTWFHAISGTN |
| | GTKRFDNPVLPFNDGVYFASTEKSNIIRGWIFGTTLDSKTQSLLIVNNATNVVIKVCEFQFCNDPFLDV |
| | YYHKNNKSWMESEFRVYSSANNCTFEYVSQPFLMDLEGKQGNFKNLREFVFKNIDGYFKIYSKHTPINL |
| | GRDLPQGFSALEPLVDLPIGINITRFQTLLALHRSYLTPGDSSSGWTAGAAAYYVGYLQPRTFLLKYNE |
| | NGTITDAVDCALDPLSETKCTLKSFTVEKGIYQTSNFRVQPTESIVRFPNITNLCPFDEVFNATTFASV |
| | YAWNRKRISNCVADYSVLYNFAPFFAFKCYGVSPTKLNDLCFTNVYADSFVIRGNEVSQIAPGQTGNIA |
| | DYNYKLPDDFTGCVIAWNSNKLDSKVGGNYNYRYRLFRKSNLKPFERDISTEIYQAGNKPCNGVAGVNC |
| | YFPLQSYGFRPTYGVGHQPYRVVVLSFELLHAPATVCGPKKSTNLVKNKCVNFNFNGLTGTGVLTESNK |
| | KFLPFQQFGRDIADTTDAVRDPQTLEILDITPCSFGGVSVITPGTNTSNQVAVLYQGVNCTEVPVAIHA |
| | DQLTPTWRVYSTGSNVFQTRAGCLIGAEYVNNSYECDIPIGAGICASYQTQTKSHRRARSVASQSIIAY |
| | TMSLGAENSVAYSNNSIAIPTNFTISVTTEILPVSMTKTSVDCTMYICGDSTECSNLLLQYGSFCTQLK |
| | RALTGIAVEQDKNTQEVFAQVKQIYKTPPIKYFGGFNFSQILPDPSKPSKRSFIEDLLFNKVTLADAGE |
| | IKQYGDCLGDIAARDLICAQKFNGLTVLPPLLTDEMIAQYTSALLAGTITSGWTFGAGAALQIPFAMQM |
| | AYRFNGIGVTQNVLYENQKLIANQFNSAIGKIQDSLSSTASALGKLQDVVNHNAQALNTLVKQLSSKFG |
| | AISSVLNDILSRLDKVEAEVQIDRLITGRLQSLQTYVTQQLIRAAEIRASANLAATKMSECVLGQSKRV |
| | DFCGKGYHLMSFPQSAPHGVVFLHVTYVPAQEKNFTTAPAICHDGKAHFPREGVFVSNGTHWFVTQRNF |
| | YEPQIITTDNTFVSGNCDVVIGIVNNTVYDPLQPELDSFKEELDKYFKNHTSPDVDLGDISGINASVVN |
| | IQKEIDRLNEVAKNLNESLIDLQELGKYEQYIKWPWYIWLGFIAGLIAIVMVTIMLCCMTSCCSCLKGC |
| | CSCGSCCKFDEDDSEPVLKGVKLHYT |

### DETAILED DESCRIPTION

Monoclonal antibodies that specifically bind SARS-CoV-2, are disclosed herein. In some embodiments, the antibodies bind a BA.4 or BA.5 variant. In some embodiments, these antibodies are of use for inhibiting a SARS-CoV-2 infection, and for detecting SARS-CoV-2 in a biological sample. The antibodies are potent neutralizing antibodies and target unique epitopes in the spike glycoprotein of SARS-CoV-2.

Worldwide genomic sequencing has revealed the occurrence of SARS-CoV-2 variants that increase transmissibility and reduce potency of vaccine-induced and therapeutic antibodies (see, for example, Wibmer et al., Nat. Med. 27, 622-625 (2021); Wang et al., Nature. 593, 130-135 (2021); Muik et al., Science. 371, 1152-1153 (2021); Wang et al., Nature. 592, 616-622 (2021)). Recently, there has been a significant concern that antibody responses to natural infection and vaccination using ancestral spike sequences may result in focused responses that lack potency against mutations present in more recent variants (e.g., K417N, LA52R, T478K, E484K/Q, N501Y in B.1.351, B.1.617.1 and B.1.617.2) (see, for example, Wibmer et al., Nat. Med. 27, 622-625 (2021); Wang et al., Nature. 593, 130-135 (2021); Muik et al., Science. 371, 1152-1153 (2021); Wang et al., Nature. 592, 616-622 (2021)). Additionally, neutralization of P.1 viruses can be achieved using sera obtained from subjects infected by B.1.351 (Moyo-Gwete et al., N. Engl. J. Med. 2 (2021), doi: 10.1056/NEJMc2104192), suggesting that shared epitopes in RBD (i.e., K417N, E484K, N501Y) are mediating the cross-reactivity. While the mechanism of B.1.351 and P.1 cross reactivity is likely focused on the 3 RBD mutations, the mechanism of broadly neutralizing antibody responses between WA-1 and later variants is not as well established. It is disclosed herein that antibodies were isolated and defined with neutralization breadth covering newly emerging SARS-CoV-2 variants, including, but not limited to, the highly transmissible variants BA.4 and BA.5.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as antibody) is administered by introducing the composition into a blood vessel of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody and Antigen Binding Fragment: An immunoglobulin, antigen-binding fragment, or derivative thereof, that specifically binds and recognizes an analyte (antigen) such as a coronavirus spike protein, such as a spike protein from SARS-CoV-2, for example, a BA.4 variant and/or a BA.5 variant. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} xd ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “VH” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273 (4): 927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27 (1): 55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L} CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some embodiments, a disclosed antibody includes a heterologous constant domain. For example, the antibody includes a constant domain that is different from a native constant domain, such as a constant domain including one or more modifications (such as the “LS” mutation) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23:1117-1125, 2005; Lonenberg, Curr. Opin. Immunol., 20:450-459, 2008).

Antibody or antigen binding fragment that neutralizes SARS-CoV-2: An antibody or antigen binding fragment that specifically binds to a SARS-CoV-2 antigen (such as the spike protein) in such a way as to inhibit a biological function associated with SARS-CoV-2 that inhibits infection. The antibody can neutralize the activity of SARS-CoV-2. For example, an antibody or antigen binding fragment that neutralizes SARS-CoV-2 may interfere with the virus by binding it directly and limiting entry into cells. Alternately, an antibody may interfere with one or more post-attachment interactions of the pathogen with a receptor, for example, by interfering with viral entry using the receptor. In some examples, an antibody that is specific for a coronavirus spike protein neutralizes the infectious titer of SARS-CoV-2.

In some embodiments, an antibody or antigen binding fragment that specifically binds to SARS-CoV-2 and neutralizes SARS-CoV-2 inhibits infection of cells, for example, by at least 50% compared to a control antibody or antigen binding fragment.

A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to a coronavirus, the antibody can bind to and inhibit the function of an antigen, such as the spike protein from coronaviruses including SARS-CoV-2.

Biological sample: A sample obtained from a subject. Biological samples include all clinical samples useful for detection of disease or infection in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a SARS-CoV-2 infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conditions sufficient to form an immune complex: Conditions which allow an antibody or antigen binding fragment to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one embodiment, an antibody is linked to an effector molecule; for example, an antibody that specifically binds to SARS-CoV-2 covalently linked to an effector molecule, such as a detectable label. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a SARS-CoV-2-specific antibody can include up to 1, 2, 3, 4, 5, 6, 7, 8, 9, or up to 10 conservative substitutions compared to a reference antibody sequence and retain specific binding activity for spike protein binding, and/or SARS-CoV-2 neutralization activity. The term conservative variation also includes the use of a substituted amino acid in place of an unsubstituted parent amino acid. Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to a coronavirus spike protein. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient not infected a coronavirus. In other embodiments, the control is a positive control, such as a tissue sample obtained from a patient diagnosed with a coronavirus infection. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Coronavirus: A family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future.

Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

Non-limiting examples of SARS-CoV-2 variants include BA.4 and BA.5. BA.4 and BA.5 have changes relative to the BA. I and BA.2 sub-lineages including the L452R and F486V mutations and the R493Q reversion in the spike receptor binding domain (RBD).

The viral genome is capped, polyadenylated, and covered with nucleocapsid proteins. The coronavirus virion includes a viral envelope containing type I fusion glycoproteins referred to as the spike(S) protein. Most coronaviruses have a common genome organization with the replicase gene.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

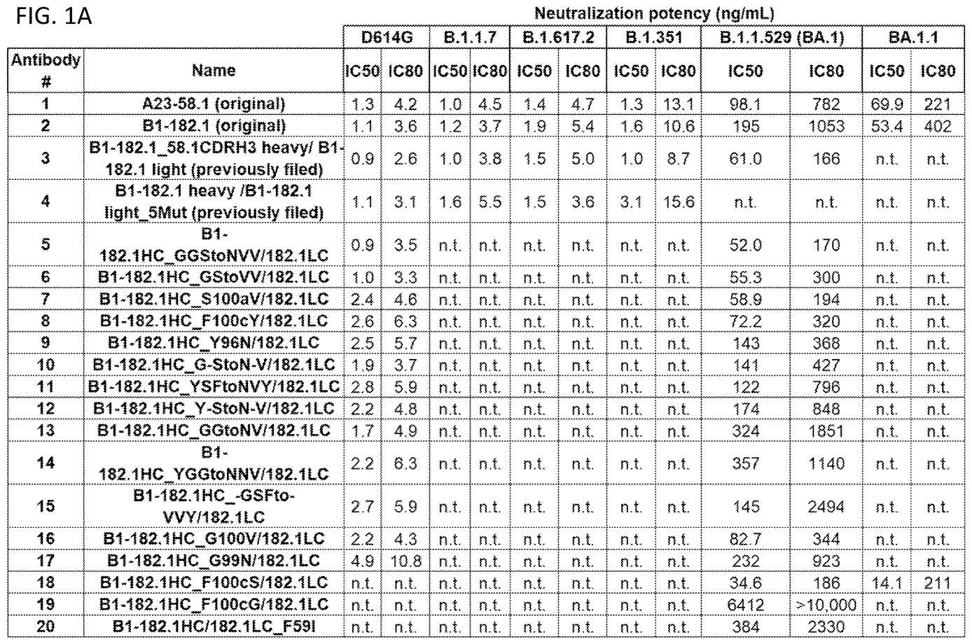

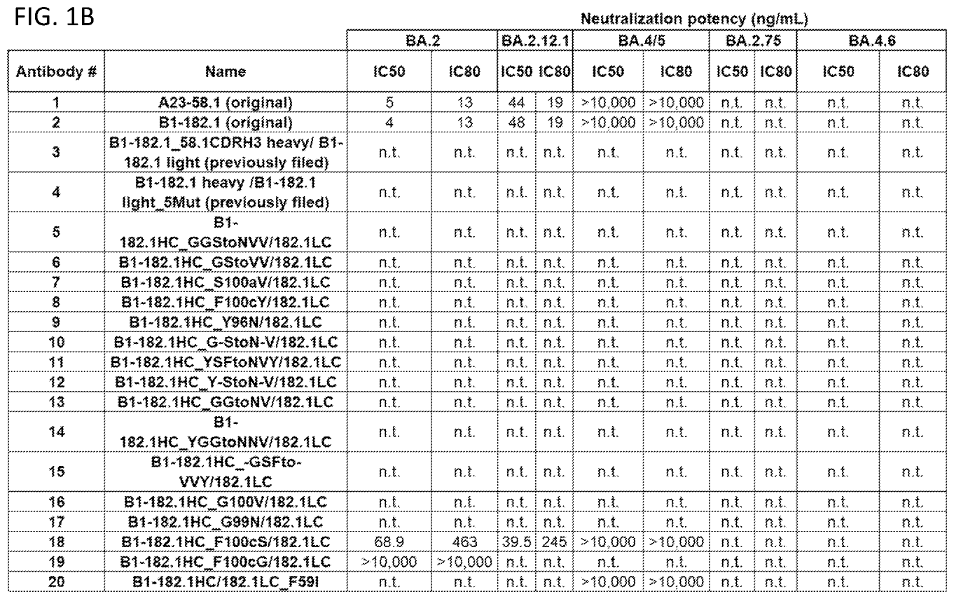

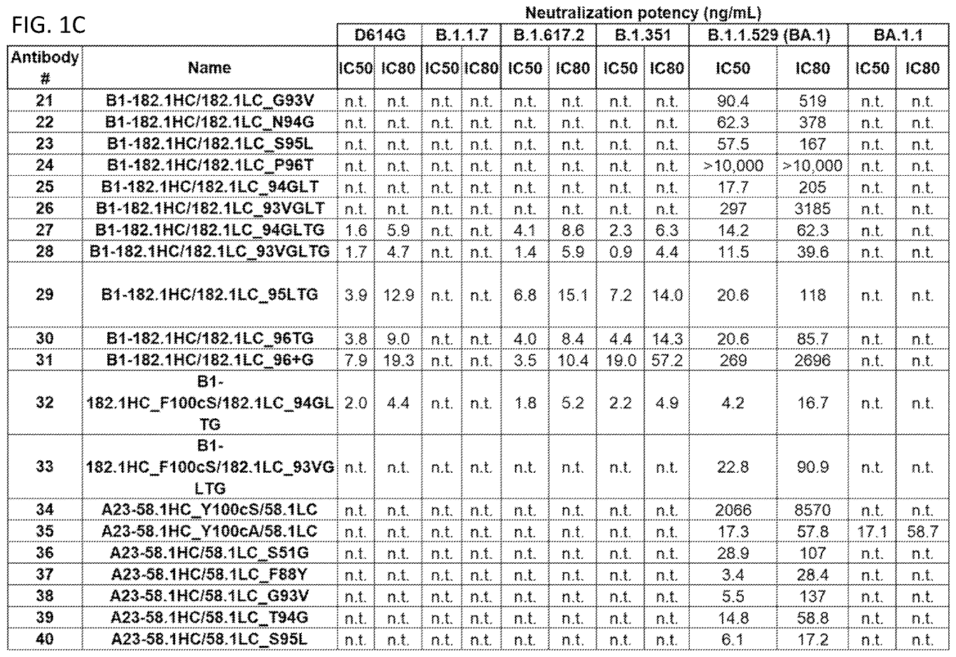

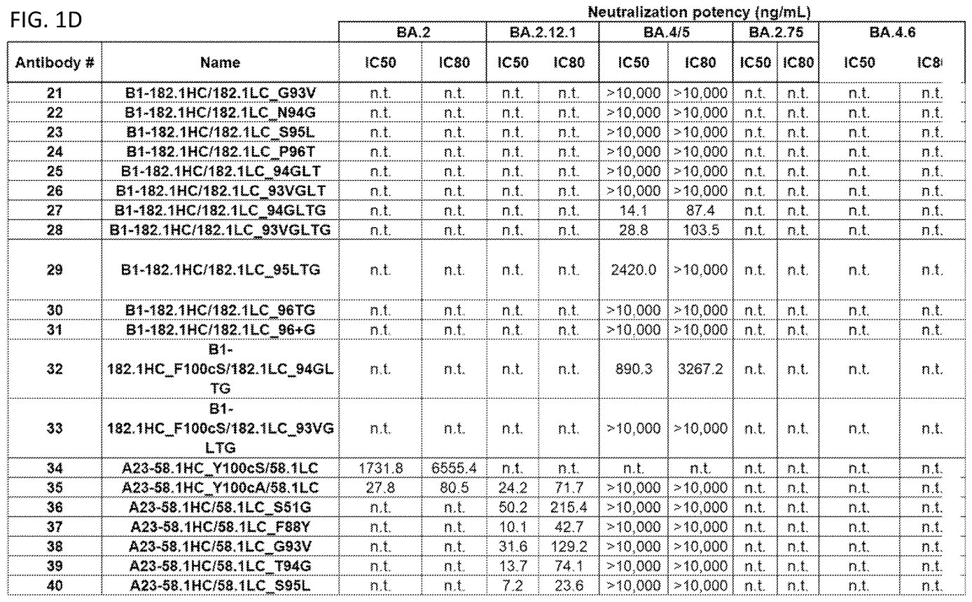

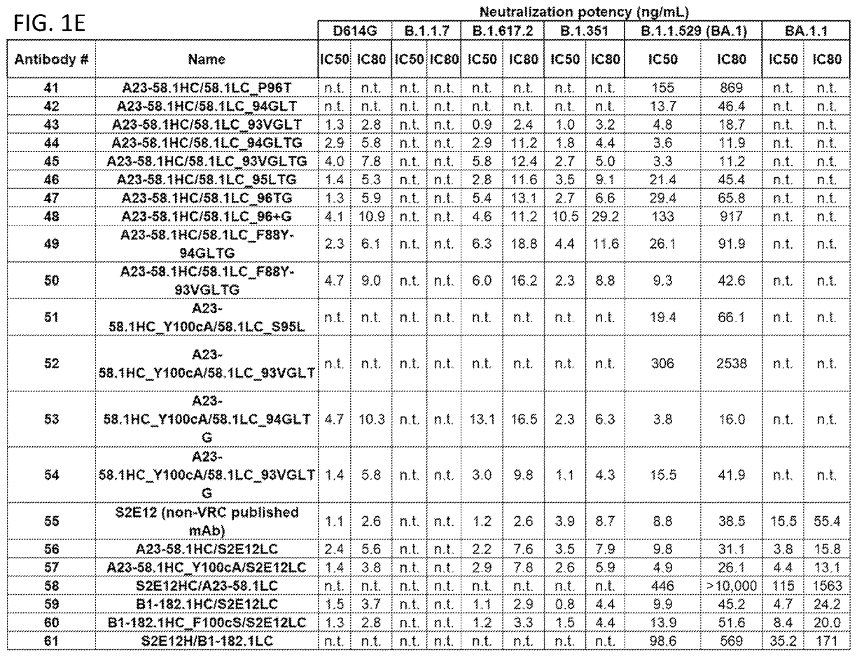

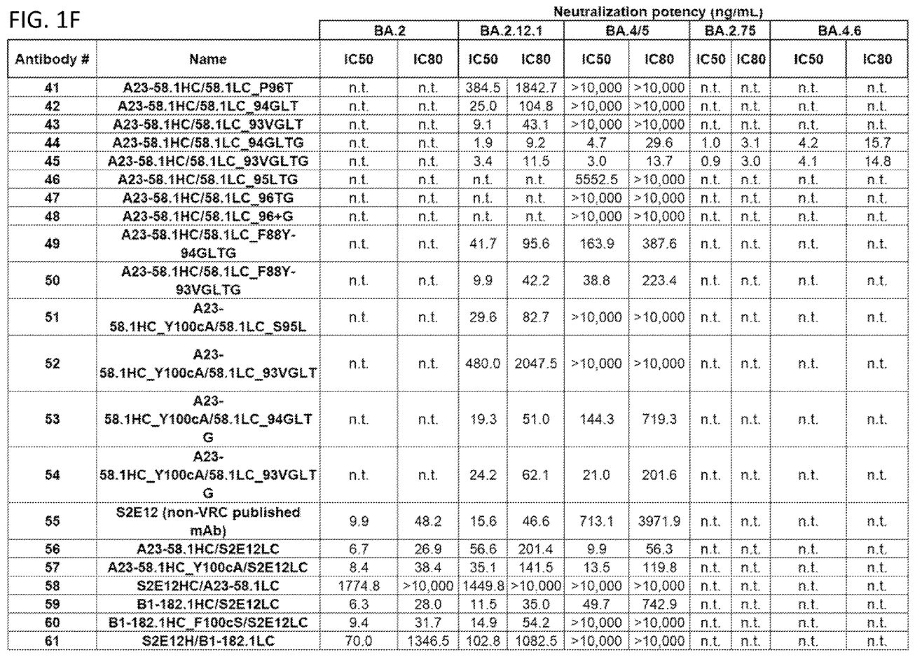

Dual variable domain immunoglobulin: A bi-specific antibody that includes two heavy chain variable domains and two light chain variable domains. Unlike IgG, however, both heavy and light chains of a DVD-immunoglobulin molecule contain an additional variable domain (VD) connected via a linker sequence at the N-termini of the VH and VL of an existing monoclonal antibody (mAb). Thus, when the heavy and the light chains combine, the resulting DVD-immunoglobulin molecule contains four antigen recognition sites, see Jakob et al., Mabs 5:358-363, 2013, incorporated herein by reference, see FIG. 1 of Jaakob et al. for schematic and space-filling diagrams. A DVD-Ig™ molecule functions to bind two different antigens on each DFab simultaneously.

Effective amount: A quantity of a specific substance sufficient to achieve a desired effect in a subject to whom the substance is administered. For instance, this can be the amount necessary to inhibit a coronavirus infection, such as a SARS-CoV-2 infection, or to measurably alter outward symptoms of such an infection.

In one example, a desired response is to inhibit or reduce or prevent SARS-CoV-2 infection. The SARS-CoV-2 infection does not need to be completely eliminated or reduced or prevented for the method to be effective.

In some embodiments, administration of an effective amount of a disclosed antibody or antigen binding fragment that binds to a coronavirus spike protein can reduce or inhibit a SAR-CoV-2 infection (for example, as measured by infection of cells, or by number or percentage of subjects infected by the coronavirus or by an increase in the survival time of infected subjects, or reduction in symptoms associated with the infection) by a desired amount, for example by at least 10%, at least 20%, at least 50%, at least 60%, at least 70%, at least 80%, at least 90%, at least 95%, at least 98%, or even at least 100% (elimination or prevention of detectable infection), as compared to a suitable control.

The effective amount of an antibody or antigen binding fragment that specifically binds the coronavirus spike protein that is administered to a subject to inhibit infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a coronavirus spike protein.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2.

Although the boundaries of the Fc region may vary, the human IgG heavy chain Fc region is usually defined to include residues following C226 or P230 to the Fc carboxyl-terminus, wherein the numbering is according to the EU numbering system. The residues can also be identified by Kabat position. For IgA, the Fc region includes immunoglobulin domains Cα2 and Cα3 and optionally the lower part of the hinge between Cα1 and Cα2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are known, for example transformation with a nucleic acid, including electroporation, lipofection, particle gun acceleration, and homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgA: A polypeptide belonging to the class of antibodies that are substantially encoded by a recognized immunoglobulin alpha gene. In humans, this class or isotype comprises IgA1 and IgA2. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG1, IgG2, IgG3, and IgG4.

Immune complex: The binding of antibody or antigen binding fragment (such as a scFv) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting or treating a disease: Inhibiting the full development of a disease or condition, for example, in a subject who is at risk for a disease such as a SARS-CoV-2 infection. “Treatment” refers to a therapeutic intervention that ameliorates a sign or symptom of a disease or pathological condition after it has begun to develop. The term “ameliorating.” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% the infection in the absence of the agent, or in comparison to a reference agent.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

SARS-CoV-2: Also known as Wuhan coronavirus or 2019 novel coronavirus, SARS-CoV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike(S)-envelope (E)-membrane (M) and nucleocapsid (N)-3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms and background and genetic tests such as reverse transcription-polymerase chain reaction (rRT-PCR). The test can be done on patient samples such as respiratory or blood samples.

B.1.1.529, also known as the omicron variant, is a variant of the original SARS-CoV-2 first reported to the World Health Organization on Nov. 21, 2021. This variant has a total of 60 mutations compared to the original strain of SARS-CoV-2, specifically 50 nonsynonymous mutations, 8 synonymous mutations, and 2 non-coding mutations. Thirty-two mutations affect the spike protein (A67V, 469-70, T95I, G142D, A143-145, A211, L212I, ins214EPE, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F), or which approximately half are located in the receptor binding domain (319-541).

The BA.4 and BA.5 sub-lineages of B.1.1.529, which do not differ in their spike sequence from each other, were also first detected by genomic surveillance in South Africa. BA.4 and BA.5 have changes relative to B.1.1.529 including the L452R and F486V mutations and the R493Q reversion in the spike receptor binding domain (RBD). BA.4 and BA.5 also differ from the BA.2 sub-lineage by a deletion of spike residues 69 and 70 (Khan et al., Nature Comm. 13, Article number 4686, doi.org/10.1038/s41467-022-32396-9, (2022).

SARS Spike(S) protein: A class I fusion glycoprotein initially synthesized as a precursor protein of approximately 1256 amino acids in size for SARS-CoV, and 1273 for SARS-CoV-2. Individual precursor S polypeptides form a homotrimer and undergo glycosylation within the Golgi apparatus as well as processing to remove the signal peptide, and cleavage by a cellular protease between approximately position 679/680 for SARS-CoV, and 685/686 for SARS-CoV-2, to generate separate S1 and S2 polypeptide chains, which remain associated as S1/S2 protomers within the homotrimer and is therefore a trimer of heterodimers. The S1 subunit is distal to the virus membrane and contains the N-terminal domain (NTD) and the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain.

The numbering used in the disclosed SARS-CoV-2 S proteins and fragments thereof is relative to the S protein of SARS-CoV-2, the sequence of which was deposited as NCBI Ref. No. YP_009724390.1, which is incorporated by reference herein in its entirety.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2 (4): 482-489, 1981; Needleman and Wunsch, J. Mol. Biol. 48 (3): 443-453, 1970; Pearson and Lipman, Proc. Natl. Acad. Sci. U.S.A. 85 (8): 2444-2448, 1988; Higgins and Sharp, Gene, 73 (1): 237-244, 1988; Higgins and Sharp, Bioinformatics, 5 (2): 151-3, 1989; Corpet, Nucleic Acids Res. 16 (22): 10881-10890, 1988; Huang et al. Bioinformatics, 8 (2): 155-165, 1992; and Pearson, Methods Mol. Biol. 24:307-331, 1994., Altschul et al., J. Mol. Biol. 215 (3): 403-410, 1990, presents a detailed consideration of sequence alignment methods and homology calculations. The NCBI Basic Local Alignment Search Tool (BLAST) (Altschul et al., J. Mol. Biol. 215 (3): 403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example a coronavirus spike protein and does not bind in a significant amount to other proteins present in the sample or subject. With regard to a spike protein, the epitope may be present on SARS-CoV-2 spike protein, such that the antibody binds to the spike protein on both types of virus, but does not bind to other proteins. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10-10 Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on SARS-CoV-2 spike protein, such as the NTD or RBD of a spike protein from SARS-CoV-2, including viruses, substrate to which the spike protein is attached, or the protein in a biological specimen. It is, of course, recognized that a certain degree of non-specific interaction may occur between an antibody and a non-target. Typically, specific binding results in a much stronger association between the antibody and a spike protein than between the antibody other different coronavirus proteins (such as MERS), or from non-coronavirus proteins. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. In some embodiments, the disclosed monoclonal antibody specifically bind the BA.4 and/or BA.5 variants of SARS-CoV-2.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a human. In an additional example, a subject is selected that is in need of inhibiting a SARS-CoV-2 infection. For example, the subject is either uninfected and at risk of the SARS-CoV-2 infection or is infected and in need of treatment.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some embodiments, a viral vector comprises a nucleic acid molecule encoding a disclosed antibody or antigen binding fragment that specifically binds to a coronavirus spike protein and neutralizes the coronavirus. In some embodiments, the viral vector can be an adeno-associated virus (AAV) vector.

Under conditions sufficient for: A phrase that is used to describe any environment that permits a desired activity.

### II. Description of Several Embodiments

Isolated monoclonal antibodies and antigen binding fragments that specifically bind a coronavirus spike protein are provided. The antibodies and antigen binding fragments can be fully human. The antibodies and antigen binding fragments can neutralize SARS-CoV-2, such as a BA.4 variant and/or a BA.5 variant. In some embodiments the disclosed antibodies can inhibit a SARS-CoV-2infection in vivo, and can be administered prior to, or after, an infection with SARS-CoV-2, such as a BA.4 variant and/or a BA.5 variant.

Bispecific antibodies including the variable domains of these antibodies are also provided. In addition, disclosed herein are compositions comprising the antibodies and antigen binding fragments and a pharmaceutically acceptable carrier.

Nucleic acids encoding the antibodies, antigen binding fragments, variable domains, and expression vectors (such as adeno-associated virus (AAV) viral vectors) comprising these nucleic acids are also provided.

The antibodies, antigen binding fragments, nucleic acid molecules, host cells, and compositions can be used for research, diagnostic, treatment and prophylactic purposes. For example, the disclosed antibodies and antigen binding fragments can be used to diagnose a subject with a SARS-CoV-2 infection or can be administered to inhibit a coronavirus infection in a subject. Binding characteristics of each of the antibodies listed below are also provided in the Examples section.

A. Monoclonal Antibodies that Specifically Bind a Coronavirus Spike Protein and Antigen Binding Fragments Thereof

The discussion of monoclonal antibodies below refers to isolated monoclonal antibodies that include heavy and/or light chain variable domains (or antigen binding fragments thereof) comprising a CDR1, CDR2, and/or CDR3 with reference to the IMGT numbering scheme (unless the context indicates otherwise). Various CDR numbering schemes (such as the Kabat, Chothia or IMGT numbering schemes) can be used to determine CDR positions. The amino acid sequence and the CDRs of the heavy and light chain of the disclosed monoclonal antibody according to the IMGT numbering scheme are provided in the listing of sequences, but these are exemplary only.

In some embodiments, a monoclonal antibody is provided that comprises the heavy and light chain CDRs of any one of the antibodies described herein. In some embodiment, a monoclonal antibody is provided that comprises the heavy and light chain variable regions of any one of the antibodies described herein. Table B provides the antibody names, numbers, and the SEQ ID NO corresponding to the sequences contained in the V_{H}, HCDR1, HCDR2, HCDR3, V_{L}, LCDR1, LCDR2, and LCDR3 for the antibodies disclosed herein. The amino acid sequences corresponding to each SEQ ID NO are provided above in Table A.

TABLE B
Antibody names, numbers, and the SEQ ID NO corresponding to the sequences contained
in the V_{H}, HCDR1, HCDR2, HCDR3, V_{L}, LCDR1, LCDR2, and LCDR3 of each.

| | | SEQ ID NO: | | | | | | | |
|---|---|---|---|---|---|---|---|---|---|
| No. | Antibody Name | V_{H} | HCDR1 | HCDR2 | HCDR3 | V_{L} | LCDR1 | LCDR2 | LCDR3 |
| 27 | B1-182.1HC/182.1LC_94GLTG | 1 | 2 | 3 | 4 | 5 | 6 | GAS | 7 |
| 28 | B1-182.1HC/182.1LC_93VGLTG | 1 | 2 | 3 | 4 | 8 | 6 | GAS | 9 |
| 44 | A23-58.1HC/58.1LC_94GLTG | 10 | 2 | 3 | 11 | 12 | 6 | SAS | 7 |
| 45 | A23-58.1HC/58.1LC_93VGLTG | 10 | 2 | 3 | 11 | 13 | 6 | SAS | 9 |
| 50 | A23-58.1HC/58.1LC_F88Y-93VGLTG | 10 | 2 | 3 | 11 | 14 | 6 | SAS | 9 |
| 54 | A23-58.1HC_Y100cA/58.1LC_93VGLTG | 15 | 2 | 3 | 16 | 13 | 6 | SAS | 9 |
| 56 | A23-58.1HC/S2E12LC | 10 | 2 | 3 | 11 | 17 | 6 | GAS | 9 |
| 57 | A23-58.1HC_Y100cA/S2E12LC | 15 | 2 | 3 | 16 | 17 | 6 | GAS | 9 |
| 59 | B1-182.1HC/S2E12LC | 1 | 2 | 3 | 4 | 17 | 6 | GAS | 9 |
| 5 | B1-182.1HC_GGStoNVV/182.1LC | 18 | 2 | 3 | 19 | 20 | 6 | GAS | 21 |
| 6 | B1-182.1HC_GStoVV/182.1LC | 22 | 2 | 3 | 23 | 20 | 6 | GAS | 21 |
| 7 | B1-182.1HC_S100aV/182.1LC | 24 | 2 | 3 | 25 | 20 | 6 | GAS | 21 |
| 8 | B1-182.1HC_F100cY/182.1LC | 26 | 2 | 3 | 27 | 20 | 6 | GAS | 21 |
| 9 | B1-182.1HC_Y96N/182.1LC | 28 | 2 | 3 | 29 | 20 | 6 | GAS | 21 |
| 10 | B1-182.1HC_G-StoN-V/182.1LC | 30 | 2 | 3 | 31 | 20 | 6 | GAS | 21 |
| 11 | B1-182.1HC_YSFtoNVY/182.1LC | 32 | 2 | 3 | 33 | 20 | 6 | GAS | 21 |
| 12 | B1-182.1HC_Y-StoN-V/182.1LC | 34 | 2 | 3 | 35 | 20 | 6 | GAS | 21 |
| 13 | B1-182.1HC_GGtoNV/182.1LC | 36 | 2 | 3 | 37 | 20 | 6 | GAS | 21 |
| 14 | B1-182.1HC_YGGtoNNV/182.1LC | 38 | 2 | 91 | 39 | 20 | 6 | GAS | 21 |
| 15 | B1-182.1HC_-GSFto-VVY/182.1LC | 40 | 2 | 3 | 41 | 20 | 6 | GAS | 21 |
| 16 | B1-182.1HC_G100V/182.1LC | 42 | 2 | 3 | 43 | 20 | 6 | GAS | 21 |
| 17 | B1-182.1HC_G99N/182.1LC | 44 | 2 | 3 | 45 | 20 | 6 | GAS | 21 |
| 18 | B1-182.1HC_F100cS/182.1LC | 46 | 2 | 3 | 47 | 20 | 6 | GAS | 21 |
| 21 | B1-182.1HC/182.1LC_G93V | 1 | 2 | 3 | 4 | 50 | 6 | GAS | 51 |
| 22 | B1-182.1HC/182.1LC_N94G | 1 | 2 | 3 | 4 | 52 | 6 | GAS | 53 |
| 23 | B1-182.1HC/182.1LC_S95L | 1 | 2 | 3 | 4 | 54 | 6 | GAS | 55 |
| 25 | B1-182.1HC/182.1LC_94GLT | 1 | 2 | 3 | 4 | 58 | 6 | GAS | 59 |
| 29 | B1-182.1HC/182.1LC_95LTG | 1 | 2 | 3 | 4 | 62 | 6 | GAS | 63 |
| 30 | B1-182.1HC/182.1LC_96TG | 1 | 2 | 3 | 4 | 64 | 6 | GAS | 65 |
| 31 | B1-182.1HC/182.1LC_96 + G | 1 | 2 | 3 | 4 | 66 | 6 | GAS | 67 |
| 32 | B1-182.1HC_F100cS/182.1LC_94GLTG | 46 | 2 | 3 | 47 | 5 | 6 | GAS | 7 |
| 33 | B1-182.1HC_F100cS/182.1LC_93VGLTG | 46 | 2 | 3 | 47 | 8 | 6 | GAS | 9 |
| 34 | A23-58.1HC_Y100cS/58.1LC | 68 | 2 | 3 | 69 | 70 | 6 | SAS | 71 |
| 35 | A23-58.1HC_Y100cA/58.1LC | 15 | 2 | 3 | 16 | 70 | 6 | SAS | 71 |
| 36 | A23-58.1HC/58.1LC_S51G | 10 | 2 | 3 | 11 | 72 | 6 | GAS | 71 |
| 37 | A23-58.1HC/58.1LC_F88Y | 10 | 2 | 3 | 11 | 73 | 6 | SAS | 71 |
| 38 | A23-58.1HC/58.1LC_G93V | 10 | 2 | 3 | 11 | 74 | 6 | SAS | 75 |
| 39 | A23-58.1HC/58.1LC_T94G | 10 | 2 | 3 | 11 | 76 | 6 | SAS | 53 |
| 40 | A23-58.1HC/58.1LC_S95L | 10 | 2 | 3 | 11 | 77 | 6 | SAS | 78 |
| 41 | A23-58.1HC/58.1LC_P96T | 10 | 2 | 3 | 11 | 79 | 6 | SAS | 80 |
| 42 | A23-58.1HC/58.1LC_94GLT | 10 | 2 | 3 | 11 | 81 | 6 | SAS | 59 |
| 43 | A23-58.1HC/58.1LC_93VGLT | 10 | 2 | 3 | 11 | 82 | 6 | SAS | 61 |
| 46 | A23-58.1HC/58.1LC_95LTG | 10 | 2 | 3 | 11 | 83 | 6 | SAS | 84 |
| 47 | A23-58.1HC/58.1LC_96TG | 10 | 2 | 3 | 11 | 85 | 6 | SAS | 86 |
| 49 | A23-58.1HC/58.1LC_F88Y-94GLTG | 10 | 2 | 3 | 11 | 89 | 6 | SAS | 7 |
| 51 | A23-58.1HC_Y100cA/58.1LC_S95L | 15 | 2 | 3 | 16 | 77 | 6 | SAS | 78 |
| 53 | A23-58.1HC_Y100cA/58.1LC_94GLTG | 15 | 2 | 3 | 16 | 12 | 6 | SAS | 7 |
| 60 | B1-182.1HC_F100cS/S2E12LC | 46 | 2 | 91 | 47 | 17 | 6 | GAS | 9 |

In some embodiments, the monoclonal antibody has a V_{L} with the amino acid sequence of SEQ ID NO: 17 or SEQ ID NO: 20. In other embodiments the monoclonal antibody includes a Vu with the amino acid sequence of SEQ ID NO: 1, SEQ ID NO: 10 or SEQ ID NO: 15. n some embodiments, the disclosed antibody neutralizes the BA.4 and/or the or BA.5 variant. The disclosed antibodies bind the receptor binding domain of the spike protein. The antibodies provided in this disclosure are discussed in further detail below.

a. Monoclonal Antibody B1-182.1HC/182.1LC_94GLTG (Antibody 27)

In some embodiments, the antibody or antigen binding fragment is based on or derived from the B1-182.1HC/182.1LC_94GLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_94GLTG antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1 and specifically binds to a coronavirus spike, and neutralizes SARS-CoV-2 In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to a coronavirus spike protein, and neutralizes SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 5, respectively, and specifically binds to a coronavirus spike protein and neutralizes SARS-CoV-2. In some embodiments, the SARS-CoV-2 is the BA.4 and/or BA.5 variant.

In some embodiments, the antibody or antigen binding fragment comprises a V_{H} comprising a HCDR1, a HCDR2, and a HCDR3 as set forth as SEQ ID NOs: 2, 3, and 4 respectively, and/or a V_{L} comprising a LCDR1, a LCDR2, and a LCDR3 as set forth as SEQ ID NOs: 6, GAS, and 7, respectively, and specifically binds to a coronavirus spike protein, and neutralizes a SARS-CoV-2.

In some embodiments, the antibody or antigen binding fragment comprises a V_{H} comprising a HCDR1, a HCDR2, and a HCDR3 as set forth as SEQ ID NO: 2, 3, and 4, respectively, a V_{L} comprising a LCDR1, a LCDR2, and a LCDR3 as set forth as SEQ ID NO: 6, GAS, and 7, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 5, such as 95%, 96%, 97%, 98% or 99% identical to SEQ ID NO: 5, and the antibody or antigens binding fragment specifically binds to a coronavirus spike protein, and neutralizes SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a coronavirus spike protein, and neutralizes SARS-CoV-2. In more embodiments, the antibody or antigen binding fragment comprises a V_{L} comprising the amino acid sequence set forth as SEQ ID NO: 5, and specifically binds to a coronavirus spike protein, and neutralizes SARS-CoV-2. In some embodiments, the antibody or antigen binding fragment comprises a V_{H} and a V_{L} comprising the amino acid sequences set forth as SEQ ID NOs: 1, 5, respectively, and specifically binds to a coronavirus spike protein, and neutralizes SARS-CoV-2.

In some embodiments, the disclosed antibodies inhibit viral entry and/or replication.

b. Monoclonal Antibody B1-182.1HC/182.ILC_93VGLTG (Antibody 28)

In some embodiments, the antibody or antigen binding fragment is based on or derived from the B1-182.1HC/182.1LC_93VGLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_93VGLTG antibody, and specifically binds to a coronavirus spike protein, and neutralizes a coronavirus.

In some embodiments, the antibody neutralizes D614G, B.1.617.2, B.1.351 and BA.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to SARS-CoV-2 spike, and neutralizes SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 8 and specifically binds to SARS-CoV-2 spike protein, and neutralizes SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 8, respectively, and specifically binds to SARS-CoV-2 spike protein and neutralizes SARS-CoV-2. In some embodiments, the SARS-CoV-2 is the BA.4 and/or BA.5 variant.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, GAS, and 9, respectively, and specifically binds to SARS-CoV-2 spike protein, and neutralizes SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 9, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 8, such as 95%, 96%, 97%, 98% or 99% identical to SEQ ID NO: 8, and the antibody or antigens binding fragment specifically binds to SARS-CoV-2 spike protein, and neutralizes SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1 and specifically binds to SARS-CoV-2 spike protein, and neutralizes SARS-CoV-2. In more embodiments, the antibody or antigen binding fragment comprises a V_{L} comprising the amino acid sequence set forth as SEQ ID NO: 8, and specifically binds to SARS-CoV-2 spike protein, and neutralizes SARS-CoV-2. In some embodiments, the antibody or antigen binding fragment comprises a V_{H} and a V_{L} comprising the amino acid sequences set forth as SEQ ID NOs: 1 and 8, respectively, and specifically binds to SARS-CoV-2 spike protein, and neutralizes SARS-CoV-2.

In some embodiments, the disclosed antibodies inhibit viral entry and/or replication.

c. Monoclonal Antibody A23-58.1HC/58.ILC_94GLTG (Antibody 44)

In some embodiments, the antibody or antigen binding fragment is based on or derived from the A23-58.1HC/58.1LC_94GLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_94GLTG antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1 and BA.2.12.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to SARS-CoV-2 spike, and neutralizes SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 12, and specifically binds to SARS-CoV-2 spike protein, and neutralizes SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 12, respectively, and specifically binds to SARS-CoV-2 spike protein and neutralizes SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 111, respectively, and/or a V_{L} comprising a LCDR1, a LCDR2, and a LCDR3 as set forth as SEQ ID NOs: 6, SAS, and 7, respectively, and specifically binds to SARS-CoV-2 spike protein, and neutralizes SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as 2, 3, and 11, respectively, a V_{L} comprising a LCDR1, a LCDR2, and a LCDR3 as set forth as 6, SAS, and 7, respectively, wherein the V_{H} comprises an amino acid sequence at least 90% identical to SEQ ID NO: 10 such as 95%, 96%, 97%, 98% or 99% identical to SEQ ID NO: 10, and wherein the V_{L} comprises an amino acid sequence at least 90% identical to SEQ ID NO: 12, such as 95%, 96%, 97%, 98% or 99% identical to SEQ ID NO: 12, and the antibody or antigens binding fragment specifically binds to a SARS-CoV-2 spike protein, and neutralizes SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10 and specifically binds to a SARS-CoV-2 spike protein, and neutralizes SARS-CoV-2. In more embodiments, the antibody or antigen binding fragment comprises a V_{L} comprising the amino acid sequence set forth as SEQ ID NO: 12, and specifically binds to a SARS-CoV-2 spike protein, and neutralizes SARS-CoV-2. In some embodiments, the antibody or antigen binding fragment comprises a V_{H} and a V_{L} comprising the amino acid sequences set forth as SEQ ID NOs: 10 and 12, respectively, and specifically binds to a SARS-CoV-2 spike protein, and neutralizes SARS-CoV-2.

In some embodiments, the disclosed antibodies inhibit viral entry and/or replication.

d. Monoclonal Antibody A23-58.1HC/58.ILC_93VGLTG (Antibody 45)

In some embodiments, the antibody or antigen binding fragment is based on or derived from the A23-58.1HC/58.1LC_93VGLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.ILC_93VGLTG antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1 and BA.2.12.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 13, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 9, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 9, respectively, wherein the V_{H} comprises an amino acid sequence at least 90% identical to SEQ ID NO: 10 such as 95%, 96%, 97%, 98% or 99% identical to SEQ ID NO: 10, and wherein the V_{L} comprises an amino acid sequence at least 90% identical to SEQ ID NO: 13, such as 95%, 96%, 97%, 98% or 99% identical to SEQ ID NO: 13, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10 and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 13, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 13, respectively, and specifically binds to a SARS-CoV-2 spike protein, and neutralizes a SARS-CoV-2.

In some embodiments, the disclosed antibodies inhibit viral entry and/or replication.

e. Monoclonal Antibody A23-58.1HC/58.ILC_F88Y-93VGLTG (Antibody 50)

In some embodiments, the antibody or antigen binding fragment is based on or derived from the A23-58.1HC/58.1LC_F88Y-93VGLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_F88Y-93VGLTG antibody, and specifically binds to a coronavirus spike protein, and neutralizes a coronavirus. The coronavirus can be SARS-CoV-2.

In some embodiments, the antibody neutralizes variants D614G, B.1.617.2, B.1.351, BA.1 and BA.2.12.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 14 and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 14, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11 respectively, and/or a V_{L} comprising a LCDR1, a LCDR2, and a LCDR3 as set forth as SEQ ID NOs: 6, SAS, and 9, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 9, respectively, wherein the V_{H} comprises an amino acid sequence at least 90% identical to SEQ ID NO: 10 such as 95%, 96%, 97%, 98% or 99% identical to SEQ ID NO: 10, and wherein the V_{L} comprises an amino acid sequence at least 90% identical to SEQ ID NO: 14, such as 95%, 96%, 97%, 98% or 99% identical to SEQ ID NO: 14, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10 and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 14, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 14, respectively, and specifically binds to a SARS-CoV-2 spike protein, and neutralizes a SARS-CoV-2.

In some embodiments, the disclosed antibodies inhibit viral entry and/or replication.

f. Monoclonal Antibody A23-58.1HC_Y100cA/58.1LC_93VGLTG (Antibody 54)

In some embodiments, the antibody or antigen binding fragment is based on or derived from the A23-58.1HC_Y100cA/58.1LC_93VGLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC_Y100cA/58.1LC_93VGLTG antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1 and BA.2.12.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 15, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 13, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 15 and 13, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 16, respectively, and/or a V_{L} comprising a LCDR1, a LCDR2, and a LCDR3 as set forth as SEQ ID NOs: 6, SAS, and 9, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 16, respectively, a V_{L} comprising a LCDR1, a LCDR2, and a LCDR3 as set forth as SEQ ID NOs: 6, SAS, and 9, respectively, wherein the V_{H} comprises an amino acid sequence at least 90% identical to SEQ ID NO: 15, such as 95%, 96%, 97%, 98% or 99% identical to SEQ ID NO: 15, and wherein the V_{L} comprises an amino acid sequence at least 90% identical to SEQ ID NO: 13, such as 95%, 96%, 97%, 98% or 99% identical to SEQ ID NO: 13, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 15 and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 13, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 15 and 13, respectively, and specifically binds to a SARS-CoV-2 spike protein, and neutralizes a SARS-CoV-2.

In some embodiments, the disclosed antibodies inhibit viral entry and/or replication.

g. Monoclonal Antibody A23-58.1HC/S2E12LC (Antibody 56)

In some embodiments, the antibody or antigen binding fragment is based on or derived from the A23-58.1HC/S2E12LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/S2E12LC antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1, BA.1.1, BA.2 and BA.2.12.1. In some embodiments, the antibody has increased neutralization for BA.1 as compared to parental antibody A19-58.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 17, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 17, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11 respectively, and/or a V_{L} comprising a LCDR1, a LCDR2, and a LCDR3 as set forth as SEQ ID NOs: 6, GAS, and 9, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, GAS, and 9, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 17, such as 95%, 96%, 97%, 98% or 99% identical to SEQ ID NO: 17, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 17, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 17, respectively, and specifically binds to a SARS-CoV-2 spike protein, and neutralizes a SARS-CoV-2.

In some embodiments, the disclosed antibodies inhibit viral entry and/or replication.

h. Monoclonal Antibody A23-58.1HC_Y100cA/S2E12LC (Antibody 57)

In some embodiments, the antibody or antigen binding fragment is based on or derived from the A23-58.1HC_Y100cA/S2E12LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC_Y100cA/S2E12LC antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1, BA.1.1, BA.2 and BA.2.12.1. In some embodiments, the antibody has increased neutralization for BA.1 as compared to parental antibody A19-58.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 15, and specifically binds to a SARS-CoV-2 spike, and neutralizes a SARS-CoV-2. In more embodiments, the antibody or antigen binding fragment comprises a V comprising an amino acid sequence at least 90% (such as at least 95%, at least 96%, at least 97%, at least 98%, or at least 99%) identical to the amino acid sequence set forth as SEQ ID NO: 17, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 15 and 17, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 16, respectively, and/or a V_{L} comprising a LCDR1, a LCDR2, and a LCDR3 as set forth as SEQ ID NOs: 6, GAS, and 9, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 16, respectively, a V_{L} comprising a LCDR1, a LCDR2, and a LCDR3 as set forth as SEQ ID NOs: 6, GAS, and 9, respectively, wherein the V_{H} comprises an amino acid sequence at least 90% identical to SEQ ID NO: 15, such as 95%, 96%, 97%, 98% or 99% identical to SEQ ID NO: 15, and wherein the V_{L} comprises an amino acid sequence at least 90% identical to SEQ ID NO: 17, such as 95%, 96%, 97%, 98% or 99% identical to SEQ ID NO: 17, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 15 and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 17, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 15 and 17, respectively, and specifically binds to a SARS-CoV-2 spike protein, and neutralizes a SARS-CoV-2.

In some embodiments, the disclosed antibodies inhibit viral entry and/or replication.

i. Monoclonal Antibody B1-182.1HC/S2E12LC (Antibody 59)

In some embodiments, the antibody or antigen binding fragment is based on or derived from the B1-182.1HC/S2E12LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/S2E12LC antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1, BA.1.1, BA.2 and BA.2.12.1. In some embodiments, the antibody has increased neutralization of BA.1 as compared to parental antibody B1-182.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 17, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 17, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 4 respectively, and/or a V_{L} comprising a LCDR1, a LCDR2, and a LCDR3 as set forth as SEQ ID NOs: 6, GAS, and 9, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 9, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 17, such as 95%, 96%, 97%, 98% or 99% identical to SEQ ID NO: 17, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 17, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 17, respectively, and specifically binds to a SARS-CoV-2 spike protein, and neutralizes a SARS-CoV-2.

In some embodiments, the disclosed antibodies inhibit viral entry and/or replication.

j. Monoclonal Antibody B1-182.1HC_GGStoNVV/182.ILC (Antibody 5)

In some embodiments, the antibody or antigen binding fragment is based on or derived from the B1-182.1HC_GGStoNVV/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_GGStoNVV/182.1LC antibody, and specifically binds to a coronavirus spike protein, and neutralizes a coronavirus. The coronavirus can be SARS-CoV-2.

In some embodiments, this antibody neutralizes BA.1. In additional embodiments, high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 18, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 18 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 19, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 19, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 18, such as 95%, 96%, 97%, 98% or 99% identical to SEQ ID NO: 18, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 18, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 18 and 20, respectively, and specifically binds to a SARS-CoV-2 spike protein, and neutralizes a SARS-CoV-2.

In some embodiments, the disclosed antibodies inhibit viral entry and/or replication.

k. Monoclonal Antibody B1-182.1HC_GStoVV/182.ILC (Antibody 6)

In some embodiments, the antibody or antigen binding fragment is based on or derived from the B1-182.1HC_GStoVV/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_GStoVV/182.1LC antibody, and specifically binds to a coronavirus spike protein, and neutralizes a coronavirus. The coronavirus can be SARS-CoV-2.

In some embodiments, the antibody neutralizes BA.1 neutralization. In other embodiments, a high yield of this antibody can be obtained by transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 22, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 22 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 23 respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 23, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 22 such as 95%, 96%, 97%, 98% or 99% identical to SEQ ID NO: 22, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 22 and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 22 and 20, respectively, and specifically binds to a SARS-CoV-2 spike protein, and neutralizes a SARS-CoV-2.

In some embodiments, the disclosed antibodies inhibit viral entry and/or replication.

l. Monoclonal Antibody B1-182.1HC_S100aV/182.1LC (Antibody 7)

In some embodiments, the antibody or antigen binding fragment is based on or derived from the B1-182.1HC_S100aV/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_S100aV/182.1LC antibody, and specifically binds to a coronavirus spike protein, and neutralizes a coronavirus. The coronavirus can be SARS-CoV-2.

In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 24, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 24 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 25, respectively, and/or a V_{L} comprising a LCDR1, a LCDR2, and a LCDR3 as set forth as SEQ ID NOs: SEQ ID NOs: 6, GAS, and 21, respectively, and specifically binds to a SARS-CoV-2 spike protein, and neutralizes a SARS-CoV-2. The coronavirus can be SARS-CoV-2.

In some embodiments, the antibody or antigen binding fragment comprises a V_{H} comprising a HCDR1, a HCDR2, and a HCDR3 as set forth as SEQ ID NOs: 2, 3, and 25, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 24, such as 95%, 96%, 97%, 98% or 99% identical to SEQ ID NO: 24, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 24, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 24 and 20, respectively, and specifically binds to a SARS-CoV-2 spike protein, and neutralizes a SARS-CoV-2.

In some embodiments, the disclosed antibodies inhibit viral entry and/or replication.

m. Monoclonal Antibody B1-182.1HC_F100cY/182.1LC (Antibody 8)

In some embodiments, the antibody or antigen binding fragment is based on or derived from the B1-182.1HC_F100cY/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_F100cY/182.1LC antibody, and specifically binds to a coronavirus spike protein, and neutralizes a coronavirus. The coronavirus can be SARS-CoV-2.

In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 26, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 26 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 27, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 27, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 26, such as 95%, 96%, 97%, 98% or 99% identical to SEQ ID NO: 26, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 26, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a Vu and a V_{L} comprising the amino acid sequences set forth as SEQ ID NOs: 26 and 20, respectively, and specifically binds to a SARS-CoV-2 spike protein, and neutralizes a SARS-CoV-2.

In some embodiments, the disclosed antibodies inhibit viral entry and/or replication.

n. Monoclonal Antibody B1-182.1HC_Y96N/182.ILC (Antibody 9)

In some embodiments, the antibody or antigen binding fragment is based on or derived from the B1-182.1HC_Y96N/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_Y96N/182.1LC antibody, and specifically binds to a coronavirus spike protein, and neutralizes a coronavirus.

In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 28, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 28 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 29, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 19, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 28, such as 95%, 96%, 97%, 98% or 99% identical to SEQ ID NO: 28, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 28, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 28 and 20, respectively, and specifically binds to a SARS-CoV-2 spike protein, and neutralizes a SARS-CoV-2.

In some embodiments, the disclosed antibodies inhibit viral entry and/or replication.

o. Monoclonal Antibody B1-182.IHC_G-StoN-V/182.ILC (Antibody 10)

In some embodiments, the antibody or antigen binding fragment is based on or derived from the B1-182.1HC_G-StoN-V/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_G-StoN-V/182.1LC antibody, and specifically binds to a coronavirus spike protein, and neutralizes a coronavirus. The coronavirus can be SARS-CoV-2.

In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 30, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 30 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 31, respectively, and/or a V_{L} comprising a LCDR1, a LCDR2, and a LCDR3 as set forth as SEQ ID NOs: 6, GAS, and 21, respectively, and specifically binds to a coronavirus spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 31, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 30, such as 95%, 96%, 97%, 98% or 99% identical to SEQ ID NO: 30, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 30, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 30 and 20, respectively, and specifically binds to a SARS-CoV-2 spike protein, and neutralizes a SARS-CoV-2.

In some embodiments, the disclosed antibodies inhibit viral entry and/or replication.

p. Monoclonal Antibody B1-182.1HC_YSFtoNVY/182.ILC (Antibody 11)

In some embodiments, the antibody or antigen binding fragment is based on or derived from the B1-182.1HC_YSFtoNVY/182.1LC antibody, specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_YSFtoNVY/182.1LC antibody, and specifically binds to a coronavirus spike protein, and neutralizes a coronavirus. The coronavirus can be SARS-CoV-2.

In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 32, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 32 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 33, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 33, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 32, such as 95%, 96%, 97%, 98% or 99% identical to SEQ ID NO: 32, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 32, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOS: 32 and 20, respectively, and specifically binds to a SARS-CoV-2 spike protein, and neutralizes a SARS-CoV-2.

In some embodiments, the disclosed antibodies inhibit viral entry and/or replication.

q. Monoclonal Antibody B1-182.1HC_Y-StoN-V/182.ILC (Antibody 12)

In some embodiments, the antibody or antigen binding fragment is based on or derived from the B1-182.1HC_Y-StoN-V/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_Y-StoN-V/182.1LC antibody, and specifically binds to a coronavirus spike protein, and neutralizes a coronavirus. The coronavirus can be SARS-CoV-2.

In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 34, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 34 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 35, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 35, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 34, such as 95%, 96%, 97%, 98% or 99% identical to SEQ ID NO: 34, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 34, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 34 and 20, respectively, and specifically binds to a SARS-CoV-2 spike protein, and neutralizes a SARS-CoV-2.

In some embodiments, the disclosed antibodies inhibit viral entry and/or replication.

r. Monoclonal Antibody B1-182.1HC_GGtoNV/182.1LC (Antibody 13)

In some embodiments, the antibody or antigen binding fragment is based on or derived from the B1-182.1HC_GGtoNV/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_GGtoNV/182.1LC antibody, and specifically binds to a coronavirus spike protein, and neutralizes a coronavirus. The coronavirus can be SARS-CoV-2. In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 36, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 36 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 37, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 37, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 36, such as 95%, 96%, 97%, 98% or 99% identical to SEQ ID NO: 36, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 36, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 36 and 20, respectively, and specifically binds to a SARS-CoV-2 spike protein, and neutralizes a SARS-CoV-2.

In some embodiments, the disclosed antibodies inhibit viral entry and/or replication.

s. Monoclonal Antibody B1-182.1HC_YGGtoNNV/182.ILC (Antibody 14)

In some embodiments, the antibody or antigen binding fragment is based on or derived from the B1-182.1HC_YGGtoNNV/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_YGGtoNNV/182.1LC antibody, and specifically binds to a coronavirus spike protein, and neutralizes a coronavirus. The coronavirus can be SARS-CoV-2.

In some embodiments, the antibody neutralizes BA.1. In other embodiments, a high yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 38, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 38 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 91, and 39, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 91, and 39, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 38, such as 95%, 96%, 97%, 98% or 99% identical to SEQ ID NO: 38, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 38, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 38 and 20, respectively, and specifically binds to a SARS-CoV-2 spike protein, and neutralizes a SARS-CoV-2.

In some embodiments, the disclosed antibodies inhibit viral entry and/or replication.

t. Monoclonal Antibody B1-182.1HC_-GSFto-VVY/182.1LC (Antibody 15)

In some embodiments, the antibody or antigen binding fragment is based on or derived from the B1-182.1HC_-GSFto-VVY/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_-GSFto-VVY/182.1LC antibody, and specifically binds to a coronavirus spike protein, and neutralizes a coronavirus. The coronavirus can be SARS-CoV-2.

In some embodiments, the antibody neutralizes BA.1. In other embodiments, a high yield of this antibody can be obtained using transient transfection. In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 40, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 40 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 41, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 41, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 40, such as 95%, 96%, 97%, 98% or 99% identical to SEQ ID NO: 40, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 40, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 40 and 20, respectively, and specifically binds to a SARS-CoV-2 spike protein, and neutralizes a SARS-CoV-2.

In some embodiments, the disclosed antibodies inhibit viral entry and/or replication.

u. Monoclonal Antibody B1-182.1HC_G100V/182.1LC (Antibody 16)

In some embodiments, the antibody or antigen binding fragment is based on or derived from the B1-182.1HC_G100V/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_G100V/182.1LC antibody, and specifically binds to a coronavirus spike protein, and neutralizes a coronavirus. The coronavirus can be SARS-CoV-2.

In some embodiments, the antibody neutralizes BA.1. In other embodiments, an improved yield of this antibody can be obtained using transient transfection.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 42, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 42 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 43, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 43, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 42, such as 95%, 96%, 97%, 98% or 99% identical to SEQ ID NO: 42, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 42, and specifically binds to a SARS-CoV-2 spike protein, and neutralizes a SARS-CoV-2. In more embodiments, the antibody or antigen binding fragment comprises a V_{L} comprising the amino acid sequence set forth as SEW ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 42 and 20, respectively, and specifically binds to a SARS-CoV-2 spike protein, and neutralizes a SARS-CoV-2.

In some embodiments, the disclosed antibodies inhibit viral entry and/or replication.

v. Monoclonal Antibody B1-182.1HC_G99N/182.ILC (Antibody 17)

In some embodiments, the antibody or antigen binding fragment is based on or derived from the B1-182.1HC_G99N/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_G99N/182.1LC antibody, and specifically binds to a coronavirus spike protein, and neutralizes a coronavirus. The coronavirus can be SARS-CoV-2.

In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 44, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 44 and 20, respectively, and specifically binds to a SARS-CoV-2 spike protein and neutralizes a SARS-CoV-2.

In some embodiments, the antibody or antigen binding fragment comprises a Vu comprising a HCDR1, a HCDR2, and a HCDR3 as set forth as SEQ ID NOs: 2, 3, and 45, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 45, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 44, such as 95%, 96%, 97%, 98% or 99% identical to SEQ ID NO: 44, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 44, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 44 and 20, respectively, and specifically binds to a SARS-CoV-2 spike protein, and neutralizes a SARS-CoV-2.

In some embodiments, the disclosed antibodies inhibit viral entry and/or replication.

w. Monoclonal Antibody B1-182.1HC_F100cS/182.ILC (Antibody 18)

In some embodiments, the antibody or antigen binding fragment is based on or derived from the B1-182.1HC_F100cS/182.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_F100cS/182.1LC antibody, and specifically binds to a coronavirus spike protein, and neutralizes a coronavirus. The coronavirus can be SARS-CoV-2.

In some embodiments, the antibody neutralizes variants BA.1, BA.1.1, BA.2, 39.5 and BA.2.12.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 46, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 20, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 46 and 20, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 47, respectively, and/or a V_{L} comprising a LCDR1, a LCDR2, and a LCDR3 as set forth as SEQ ID NOs: 6, GAS, and 21, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 47, respectively, a V_{L} comprising a LCDR1, a LCDR2, and a LCDR3 as set forth as SEQ ID NOs: 6, GAS, and 21, respectively, wherein the V_{H} comprises an amino acid sequence at least 90% identical to SEQ ID NO: 46, such as 95%, 96%, 97%, 98% or 99% identical to SEQ ID NO: 46, and wherein the V_{L} comprises an amino acid sequence at least 90% identical to SEQ ID NO: 20, such as 95%, 96%, 97%, 98% or 99% identical to SEQ ID NO: 20, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 46, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 20, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOS: 46 and 20, respectively, and specifically binds to a SARS-CoV-2 spike protein, and neutralizes a SARS-CoV-2.

In some embodiments, the disclosed antibodies inhibit viral entry and/or replication.

x. Monoclonal Antibody B1-182.1HC/182.1LC_G93V (Antibody 21)

In some embodiments, the antibody or antigen binding fragment is based on or derived from the B1-182.1HC/182.1LC_G93V antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_G93V antibody, and specifically binds to a coronavirus spike protein, and neutralizes a coronavirus. The coronavirus can be SARS-CoV-2.

In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 50, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 50, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, GAS, and 51, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 51, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 50, such as 95%, 96%, 97%, 98% or 99% identical to SEQ ID NO: 50, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1 and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 50, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 50, respectively, and specifically binds to a SARS-CoV-2 spike protein, and neutralizes a SARS-CoV-2.

In some embodiments, the disclosed antibodies inhibit viral entry and/or replication.

y. Monoclonal Antibody B1-182.1HC/182.1LC_N94G (Antibody 22)

In some embodiments, the antibody or antigen binding fragment is based on or derived from the B1-182.1HC/182.1LC_N94G antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_N94G antibody, and specifically binds to a coronavirus spike protein, and neutralizes a coronavirus. The coronavirus can be SARS-CoV-2.

In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 52, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 52, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, GAS, and 53, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 53, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 52, such as 95%, 96%, 97%, 98% or 99% identical to SEQ ID NO: 52, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 52, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 52, respectively, and specifically binds to a SARS-CoV-2 spike protein, and neutralizes a SARS-CoV-2.

In some embodiments, the disclosed antibodies inhibit viral entry and/or replication.

z. Monoclonal Antibody B1-182.1HC/182.1LC_S95L (Antibody 23)

In some embodiments, the antibody or antigen binding fragment is based on or derived from the B1-182.1HC/182.1LC_S95L antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_S95L antibody, and specifically binds to a coronavirus spike protein, and neutralizes a coronavirus. The coronavirus can be SARS-CoV-2.

In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 54, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 54, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: SEQ ID NOs: 2, 3, and 4, respectively, and/or a V_{L} comprising a LCDR1, a LCDR2, and a LCDR3 as set forth as SEQ ID NOs: 6, GAS, and 55, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 55, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 54, such as 95%, 96%, 97%, 98% or 99% identical to SEQ ID NO: 54, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 54, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 54, respectively, and specifically binds to a SARS-CoV-2 spike protein, and neutralizes a SARS-CoV-2.

In some embodiments, the disclosed antibodies inhibit viral entry and/or replication.

aa. Monoclonal Antibody B1-182.1HC/182.ILC_94GLT (Antibody 25)

In some embodiments, the antibody or antigen binding fragment is based on or derived from the B1-182.1HC/182.1LC_94GLT antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_94GLT antibody, and specifically binds to a coronavirus spike protein, and neutralizes a coronavirus. The coronavirus can be SARS-CoV-2.

In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 58, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 58, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, GAS, and 59, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 59, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 58, such as 95%, 96%, 97%, 98% or 99% identical to SEQ ID NO: 58, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 58, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 58, respectively, and specifically binds to a SARS-CoV-2 spike protein, and neutralizes a SARS-CoV-2.

In some embodiments, the disclosed antibodies inhibit viral entry and/or replication.

bb. Monoclonal Antibody B1-182.1HC/182.ILC_95LTG (Antibody 29)

In some embodiments, the antibody or antigen binding fragment is based on or derived from the B1-182.1HC/182.1LC_95LTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_95LTG antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 62, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 62, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: SEQ ID NOs: 2, 3, and 4, respectively, and/or a V_{L} comprising a LCDR1, a LCDR2, and a LCDR3 as set forth as SEQ ID NOs: 6, GAS, and 63, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 63, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 62, such as 95%, 96%, 97%, 98% or 99% identical to SEQ ID NO: 62, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 62, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 62, respectively, and specifically binds to a SARS-CoV-2 spike protein, and neutralizes a SARS-CoV-2.

In some embodiments, the disclosed antibodies inhibit viral entry and/or replication.

cc. Monoclonal Antibody B1-182.1HC/182.ILC_96TG (Antibody 30)

In some embodiments, the antibody or antigen binding fragment is based on or derived from the B1-182.1HC/182.1LC_96TG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_96TG antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 64, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 64, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, GAS, and 65, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 65, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 64, such as 95%, 96%, 97%, 98% or 99% identical to SEQ ID NO: 64, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 64, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 64, respectively, and specifically binds to a SARS-CoV-2 spike protein, and neutralizes a SARS-CoV-2.

In some embodiments, the disclosed antibodies inhibit viral entry and/or replication.

dd. Monoclonal Antibody B1-182.1HC/182.ILC_96+G (Antibody 31)

In some embodiments, the antibody or antigen binding fragment is based on or derived from the B1-182.1HC/182.LC_96+G antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC/182.1LC_96+G antibody, and specifically binds to a coronavirus spike protein, and neutralizes a coronavirus. The coronavirus can be SARS-CoV-2.

In some embodiments, the antibody neutralizes D614G, B.1.617.2 and B.1.351. In some embodiments, the antibody neutralizes BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 1, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 66, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 1 and 66, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, and/or a V_{L} comprising a LCDR1, a LCDR2, and a LCDR3 as set forth as SEQ ID NOs: 6, GAS, and 67, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 4, respectively, a V_{L} comprising a LCDR1, a LCDR2, and a LCDR3 as set forth as SEQ ID NOs: 6, GAS, and 67, respectively, wherein the V_{H} comprises an amino acid sequence at least 90% identical to SEQ ID NO: 1, such as 95%, 96%, 97%, 98% or 99% identical to SEQ ID NO: 1, and wherein the V_{L} comprises an amino acid sequence at least 90% identical to SEQ ID NO: 66, such as 95%, 96%, 97%, 98% or 99% identical to SEQ ID NO: 66, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 1, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 66, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 1 and 66, respectively, and specifically binds to a SARS-CoV-2 spike protein, and neutralizes a SARS-CoV-2.

In some embodiments, the disclosed antibodies inhibit viral entry and/or replication.

ee. Monoclonal Antibody B1-182.1HC_F100cS/182.1LC_94GLTG (Antibody 32)

In some embodiments, the antibody or antigen binding fragment is based on or derived from the BI-182.1HC_F100cS/182.1LC_94GLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_F100cS/182.ILC_94GLTG antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1. In more embodiments, the antibody has improved neutralization for BA.1 as compared to parental antibody B1-182.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 46, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 5, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 46 and 5, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 47, respectively, and/or a V_{L} comprising a LCDR1, a LCDR2, and a LCDR3 as set forth as SEQ ID NOs: 6, GAS, and 7, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 47, respectively, a V_{L} comprising a LCDR1, a LCDR2, and a LCDR3 as set forth as SEQ ID NOs: 6, GAS, and 7, respectively, wherein the V_{H} comprises an amino acid sequence at least 90% identical to SEQ ID NO: 46, such as 95%, 96%, 97%, 98% or 99% identical to SEQ ID NO: 46, and wherein the V_{L} comprises an amino acid sequence at least 90% identical to SEQ ID NO: 5, such as 95%, 96%, 97%, 98% or 99% identical to SEQ ID NO: 5, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 46, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 5, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 46 and 5, respectively, and specifically binds to a SARS-CoV-2 spike protein, and neutralizes a SARS-CoV-2.

In some embodiments, the disclosed antibodies inhibit viral entry and/or replication.

ff. Monoclonal Antibody B1-182.1HC_F100cS/182.1LC_93VGLTG (Antibody 33)

In some embodiments, the antibody or antigen binding fragment is based on or derived from the B1-182.1HC_F100cS/182.1LC_93VGLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_F100cS/182.1LC_93VGLTG antibody, and specifically binds to a coronavirus spike protein, and neutralizes a coronavirus. The coronavirus can be SARS-CoV-2.

In some embodiments, the antibody neutralizes BA.1 more potently than parental antibody B1-182.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 46, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 8, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 46 and 8, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 47, respectively, and/or a V_{L} comprising a LCDR1, a LCDR2, and a LCDR3 as set forth as SEQ ID NOs: 6, GAS, and 9, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 47, respectively, a V_{L} comprising a LCDR1, a LCDR2, and a LCDR3 as set forth as SEQ ID NOs: 6, GAS, and 9, respectively, wherein the V_{H} comprises an amino acid sequence at least 90% identical to SEQ ID NO: 46, such as 95%, 96%, 97%, 98% or 99% identical to SEQ ID NO: 46, and wherein the V_{L} comprises an amino acid sequence at least 90% identical to SEQ ID NO: 8, such as 95%, 96%, 97%, 98% or 99% identical to SEQ ID NO: 8, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 46, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 8, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 46 and 8, respectively, and specifically binds to a SARS-CoV-2 spike protein, and neutralizes a SARS-CoV-2.

In some embodiments, the disclosed antibodies inhibit viral entry and/or replication.

gg. Monoclonal Antibody A23-58.1HC_Y100cS/58.ILC (Antibody 34)

In some embodiments, the antibody or antigen binding fragment is based on or derived from the A23-58.1HC_Y100cS/58.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC_Y100cS/58.1LC antibody, and specifically binds to a coronavirus spike protein, and neutralizes a coronavirus. The coronavirus can be SARS-CoV-2.

In some embodiments, the antibody neutralizes variant BA.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 68, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 70, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 68 and 70, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 69, respectively, and/or a V_{L} comprising a LCDR1, a LCDR2, and a LCDR3 as set forth as SEQ ID NOs: 6, SAS, and 71, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 69, respectively, a V_{L} comprising a LCDR1, a LCDR2, and a LCDR3 as set forth as SEQ ID NOs: 6, SAS, and 71, respectively, wherein the V_{H} comprises an amino acid sequence at least 90% identical to SEQ ID NO: 68, such as 95%, 96%, 97%, 98% or 99% identical to SEQ ID NO: 68, and wherein the V_{L} comprises an amino acid sequence at least 90% identical to SEQ ID NO: 70, such as 95%, 96%, 97%, 98% or 99% identical to SEQ ID NO: 70, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 68, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 70, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 68 and 70, respectively, and specifically binds to a SARS-CoV-2 spike protein, and neutralizes a SARS-CoV-2.

In some embodiments, the disclosed antibodies inhibit viral entry and/or replication.

hh. Monoclonal Antibody A23-58.1HC_Y100cA/58.ILC (Antibody 35)

In some embodiments, the antibody or antigen binding fragment is based on or derived from the A23-58.1HC_Y100cA/58.1LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC_Y100cA/58.1LC antibody, and specifically binds to a coronavirus spike protein, and neutralizes a coronavirus. The coronavirus can be SARS-CoV-2.

In some embodiments, the antibody neutralizes BA.1, BA.1.1, BA.2 and BA.2.12.1. In more embodiments, the antibody neutralizes BA.1 more potently than parental antibody A19-58.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 15, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 70, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 15 and 70, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 16, respectively, and/or a V_{L} comprising a LCDR1. a LCDR2, and a LCDR3 as set forth as SEQ ID NOs: SEQ ID NOs: 6, SAS, and 71, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 16, respectively, a V_{L} comprising a LCDR1, a LCDR2, and a LCDR3 as set forth as SEQ ID NOs: 6, SAS, and 71, respectively, wherein the V_{H} comprises an amino acid sequence at least 90% identical to SEQ ID NO: 15, such as 95%, 96%, 97%, 98% or 99% identical to SEQ ID NO: 15, and wherein the V_{L} comprises an amino acid sequence at least 90% identical to SEQ ID NO: 70, such as 95%, 96%, 97%, 98% or 99% identical to SEQ ID NO: 70, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 15, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 70, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 15 and 70, respectively, and specifically binds to a SARS-CoV-2 spike protein, and neutralizes a SARS-CoV-2.

In some embodiments, the disclosed antibodies inhibit viral entry and/or replication.

ii. Monoclonal Antibody A23-58.1HC/58.ILC_S51G (Antibody 36)

In some embodiments, the antibody or antigen binding fragment is based on or derived from the A23-58.1HC/58.1LC_S51G antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_S51G antibody, and specifically binds to a coronavirus spike protein, and neutralizes a coronavirus. The coronavirus can be SARS-CoV-2.

In some embodiments, the antibody neutralizes BA.1 more potently than parental antibody A19-58.1. In other embodiments, the antibody neutralizes BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 72, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 72, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, GAS, and 71, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, GAS, and 71, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 72, such as 95%, 96%, 97%, 98% or 99% identical to SEQ ID NO: 72, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 72, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 72, respectively, and specifically binds to a SARS-CoV-2 spike protein, and neutralizes a SARS-CoV-2.

In some embodiments, the disclosed antibodies inhibit viral entry and/or replication.

jj. Monoclonal Antibody A23-58.1HC/58.ILC_F88Y (Antibody 37)

In some embodiments, the antibody or antigen binding fragment is based on or derived from the A23-58.1HC/58.1LC_F88Y antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_F88Y antibody, and specifically binds to a coronavirus spike protein, and neutralizes a coronavirus. The coronavirus can be SARS-CoV-2.

In some embodiments, the antibody neutralizes BA.1 more potently than parental antibody A19-58.1. In some embodiments, the antibody neutralizes BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 73, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 73, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 71, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 71, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 73, such as 95%, 96%, 97%, 98% or 99% identical to SEQ ID NO: 73, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 73, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 73, respectively, and specifically binds to a SARS-CoV-2 spike protein, and neutralizes a SARS-CoV-2.

In some embodiments, the disclosed antibodies inhibit viral entry and/or replication.

kk. Monoclonal Antibody A23-58.1HC/58.ILC_G93V (Antibody 38)

In some embodiments, the antibody or antigen binding fragment is based on or derived from the A23-58.1HC/58.1LC_G93V antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_G93V antibody, and specifically binds to a coronavirus spike protein, and neutralizes a coronavirus. The coronavirus can be SARS-CoV-2.

In some embodiments, the antibody neutralizes BA.1 more potently than parental antibody A19-58.1. In some embodiments, the antibody neutralizes BA.2.12.1.

In some embodiments, the antibody or antigen binding fragment comprises a Vu comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 74, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 74, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11 respectively, and/or a V_{L} comprising a LCDR1, a LCDR2, and a LCDR3 as set forth as SEQ ID NOs: 6, SAS, and 75, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 75, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 74, such as 95%, 96%, 97%, 98% or 99% identical to SEQ ID NO: 74, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 74, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOS: 10 and 74, respectively, and specifically binds to a SARS-CoV-2 spike protein, and neutralizes a SARS-CoV-2.

In some embodiments, the disclosed antibodies inhibit viral entry and/or replication.

ll. Monoclonal Antibody A23-58.1HC/58.1LC_T94G (Antibody 39)

In some embodiments, the antibody or antigen binding fragment is based on or derived from the A23-58.1HC/58.1LC_T94G antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_T94G antibody, and specifically binds to a coronavirus spike protein, and neutralizes a coronavirus. The coronavirus can be SARS-CoV-2.

In some embodiments, the antibody neutralizes BA.1 more potently than parental antibody A19-58.1. In some embodiments, the antibody neutralizes BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 76, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 76, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 53, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 53, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 76, such as 95%, 96%, 97%, 98% or 99% identical to SEQ ID NO: 76, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 76, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 76, respectively, and specifically binds to a SARS-CoV-2 spike protein, and neutralizes a SARS-CoV-2.

In some embodiments, the disclosed antibodies inhibit viral entry and/or replication.

mm. Monoclonal Antibody A23-58.1HC/58.1LC_S95L (Antibody 40)

In some embodiments, the antibody or antigen binding fragment is based on or derived from the A23-58.1HC/58.1LC_S95L antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_S95L antibody, and specifically binds to a coronavirus spike protein, and neutralizes a coronavirus. The coronavirus can be SARS-CoV-2.

In some embodiments, the antibody neutralizes BA.1. In some embodiments, the antibody neutralizes BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 77, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 77, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 78, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 78, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 77. such as 95%, 96%, 97%, 98% or 99% identical to SEQ ID NO: 77, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 77, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOS: 10 and 77, respectively, and specifically binds to a SARS-CoV-2 spike protein, and neutralizes a SARS-CoV-2.

In some embodiments, the disclosed antibodies inhibit viral entry and/or replication.

nn. Monoclonal Antibody A23-58.1HC/58.ILC_P96T (Antibody 41)

In some embodiments, the antibody or antigen binding fragment is based on or derived from the A23-58.1HC/58.1LC_P96T antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_P96T antibody, and specifically binds to a coronavirus spike protein, and neutralizes a coronavirus. The coronavirus can be SARS-CoV-2.

In some embodiments, the antibody neutralizes BA.1 and BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 79, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 79, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 80, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 80, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 79, such as 95%, 96%, 97%, 98% or 99% identical to SEQ ID NO: 79, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 79, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOS: 10 and 79, respectively, and specifically binds to a SARS-CoV-2 spike protein, and neutralizes a SARS-CoV-2.

In some embodiments, the disclosed antibodies inhibit viral entry and/or replication.

oo. Monoclonal Antibody A23-58.IHC/58.ILC_94GLT (Antibody 42)

In some embodiments, the antibody or antigen binding fragment is based on or derived from the A23-58.1HC/58.1LC_94GLT antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_94GLT antibody, and specifically binds to a coronavirus spike protein, and neutralizes a coronavirus. The coronavirus can be SARS-CoV-2.

In some embodiments, the antibody neutralizes BA.1 more potently than parental antibody A19-58.1. In some embodiments, the antibody neutralizes BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 81 and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 81, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 59, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 59, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 81, such as 95%, 96%, 97%, 98% or 99% identical to SEQ ID NO: 81, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 81, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOS: 10 and 81, respectively, and specifically binds to a SARS-CoV-2 spike protein, and neutralizes a SARS-CoV-2.

In some embodiments, the disclosed antibodies inhibit viral entry and/or replication.

pp. Monoclonal Antibody A23-58.1HC/58.ILC_93VGLT (Antibody 43)

In some embodiments, the antibody or antigen binding fragment is based on or derived from the A23-58.1HC/58.1LC_93VGLT antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_93VGLT antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1. In some embodiments, the antibody neutralizes BA.1 more potently than parental antibody A19-58.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 82, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 82, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 61, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 61, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 82, such as 95%, 96%, 97%, 98% or 99% identical to SEQ ID NO: 82, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 82, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 82, respectively, and specifically binds to a SARS-CoV-2 spike protein, and neutralizes a SARS-CoV-2.

In some embodiments, the disclosed antibodies inhibit viral entry and/or replication.

qq. Monoclonal Antibody A23-58.1HC/58.ILC_95LTG (Antibody 46)

In some embodiments, the antibody or antigen binding fragment is based on or derived from the A23-58.1HC/58.1LC_95LTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_95LTG antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1. In some embodiments, the antibody neutralizes BA.1 more potently than parental antibody A19-58.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 83, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 83, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 84, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 84, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 83, such as 95%, 96%, 97%, 98% or 99% identical to SEQ ID NO: 83, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 83, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 83, respectively, and specifically binds to a SARS-CoV-2 spike protein, and neutralizes a SARS-CoV-2.

In some embodiments, the disclosed antibodies inhibit viral entry and/or replication.

rr. Monoclonal Antibody A23-58.1HC/58.1LC_96TG (Antibody 47)

In some embodiments, the antibody or antigen binding fragment is based on or derived from the A23-58.1HC/58.1LC_96TG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_96TG antibody, and specifically binds to a coronavirus spike protein, and neutralizes a coronavirus. The coronavirus can be SARS-CoV-2.

In some embodiments, the antibody neutralizes D614G, B.1.617.2, B.1.351 and BA.1. In some embodiments, the antibody neutralizes BA.1 more potently than parental antibody A19-58.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 85, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 85, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 86, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 86, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 85, such as 95%, 96%, 97%, 98% or 99% identical to SEQ ID NO: 85, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 85, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 85, respectively, and specifically binds to a SARS-CoV-2 spike protein, and neutralizes a SARS-CoV-2.

In some embodiments, the disclosed antibodies inhibit viral entry and/or replication.

ss. Monoclonal Antibody A23-58.1HC/58.1LC_F88Y-94GLTG (Antibody 49)

In some embodiments, the antibody or antigen binding fragment is based on or derived from the A23-58.1HC/58.1LC_F88Y-94GLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC/58.1LC_F88Y-94GLTG antibody, and specifically binds to a coronavirus spike protein, and neutralizes a coronavirus. The coronavirus can be SARS-CoV-2.

In some embodiments, the antibody neutralizes B.1.617.2, B.1.351, BA.1 and BA.2.12.1. In some embodiments, the antibody neutralizes BA.1 more potently than parental antibody A19-58.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 10, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 89, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 10 and 89, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, and/or a V_{L} comprising a LCDR1, a LCDR2, and a LCDR3 as set forth as SEQ ID NOs: 6, SAS, and 7, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 11, respectively, a V_{L} comprising a LCDR1, a LCDR2, and a LCDR3 as set forth as SEQ ID NOs: 6, SAS, and 7, respectively, wherein the V_{H} comprises an amino acid sequence at least 90% identical to SEQ ID NO: 10, such as 95%, 96%, 97%, 98% or 99% identical to SEQ ID NO: 10, and wherein the V_{L} comprises an amino acid sequence at least 90% identical to SEQ ID NO: 89, such as 95%, 96%, 97%, 98% or 99% identical to SEQ ID NO: 89, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 10, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 89, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 10 and 89, respectively, and specifically binds to a SARS-CoV-2 spike protein, and neutralizes a SARS-CoV-2.

In some embodiments, the disclosed antibodies inhibit viral entry and/or replication.

tt. Monoclonal Antibody A23-58.1HC_Y100cA/58.1LC_S95L (Antibody 51)

In some embodiments, the antibody or antigen binding fragment is based on or derived from the A23-58.1HC_Y100cA/58.1LC_S95L antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC_Y100cA/58.1LC_S95L antibody, and specifically binds to a coronavirus spike protein, and neutralizes a coronavirus. The coronavirus can be SARS-CoV-2.

In some embodiments, the antibody neutralizes BA.1 and BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 15, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 77, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 15 and 77, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 16, respectively, and/or a V_{L} comprising a LCDR1, a LCDR2, and a LCDR3 as set forth as SEQ ID NOs: 6, SAS, and 78, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 3, and 16, respectively, a V_{L} comprising a LCDR1, a LCDR2, and a LCDR3 as set forth as SEQ ID NOs: 6, SAS, and 78, respectively, wherein the V_{H} comprises an amino acid sequence at least 90% identical to SEQ ID NO: 15, such as 95%, 96%, 97%, 98% or 99% identical to SEQ ID NO: 15, and wherein the V_{L} comprises an amino acid sequence at least 90% identical to SEQ ID NO: 77, such as 95%, 96%, 97%, 98% or 99% identical to SEQ ID NO: 77, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 15, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 77, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 15 and 77, respectively, and specifically binds to a SARS-CoV-2 spike protein, and neutralizes a SARS-CoV-2.

In some embodiments, the disclosed antibodies inhibit viral entry and/or replication.

uu. Monoclonal Antibody A23-58.1HC_Y100cA/58.1LC_94GLTG (Antibody 53)

In some embodiments, the antibody or antigen binding fragment is based on or derived from the A23-58.1HC_Y100cA/58.1LC_94GLTG antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the A23-58.1HC_Y100cA/58.1LC_94GLTG antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1 and BA.2.12.1. In some embodiments, the antibody neutralizes BA.1 more potently than parental antibody A19-58.1. In some embodiments, the antibody neutralizes the BA.4 and/or BA.5 variant.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 15, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 12, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 15 and 12, respectively, and specifically binds to a SARS-CoV-2 spike protein and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 16, respectively, and/or a V_{L} comprising a LCDR1, a LCDR2, and a LCDR3 as set forth as SEQ ID NOs: 6, SAS, and 7, respectively, and specifically binds to a SARS-CoV-2 spike protein, and neutralizes a SARS-CoV-2. In some embodiments, the SARS-CoV-2 is the BA.4 or BA.5 variant.

In some embodiments, the antibody or antigen binding fragment comprises a V_{H} comprising a HCDR1, a HCDR2, and a HCDR3 as set forth as SEQ ID NOs: 2, 3, and 16, respectively, a V_{L} comprising a LCDR1, a LCDR2, and a LCDR3 as set forth as SEQ ID NOs: 6, SAS, and 7, respectively, wherein the V_{H} comprises an amino acid sequence at least 90% identical to SEQ ID NO: 15, such as 95%, 96%, 97%, 98% or 99% identical to SEQ ID NO: 15, and wherein the V_{L} comprises an amino acid sequence at least 90% identical to SEQ ID NO: 12, such as 95%, 96%, 97%, 98% or 99% identical to SEQ ID NO: 12, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 15, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 12, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 15 and 12, respectively, and specifically binds to a SARS-CoV-2 spike protein, and neutralizes a SARS-CoV-2.

In some embodiments, the disclosed antibodies inhibit viral entry and/or replication.

vv. Monoclonal Antibody B1-182.1HC_F100cS/S2E12LC (Antibody 60)

In some embodiments, the antibody or antigen binding fragment is based on or derived from the B1-182.1HC_F100cS/S2E12LC antibody, and specifically binds to a coronavirus spike protein, and neutralizes a coronavirus.

In some examples, the antibody or antigen binding fragment comprises a V_{H} and a V_{L} comprising the HCDR1, the HCDR2, the HCDR3, the LCDR1, the LCDR2, and the LCDR3, respectively (for example, according to IMGT, Kabat or Chothia), of the B1-182.1HC_F100cS/S2E12LC antibody, and specifically binds to a coronavirus spike protein, and neutralizes a coronavirus. The coronavirus can be SARS-CoV-2.

In some embodiments, the antibody neutralizes D614G, B.1.617.2, B.1.351, BA.1, BA.1.1, BA.2 and BA.2.12.1.

In some embodiments, the antibody or antigen binding fragment comprises a V_{H} comprising an amino acid sequence at least 90% (such as at least 95%, at least 96%, at least 97%, at least 98%, or at least 99%) identical to the amino acid sequence set forth as SEQ ID NO: 46, and specifically binds to a SARS-CoV-2 spike, and neutralizes a SARS-CoV-2. In more embodiments, the antibody or antigen binding fragment comprises a V_{L} comprising an amino acid sequence at least 90% (such as at least 95%, at least 96%, at least 97%, at least 98%, or at least 99%) identical to the amino acid sequence set forth as SEQ ID NO: 17, and specifically binds to a SARS-CoV-2 spike protein, and neutralizes a SARS-CoV-2. In additional embodiments, the antibody or antigen binding fragment comprises a V_{H} and a V_{L} independently comprising amino acid sequences at least 90% (such as at least 95%, at least 96%, at least 97%, at least 98%, or at least 99%) identical to the amino acid sequences set forth as SEQ ID NOs: 46 and 17, respectively, and specifically binds to a SARS-CoV-2 spike protein and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 91, and 47, respectively, and/or a V_{L} comprising a LCDR1, a LCDR2, and a LCDR3 as set forth as SEQ ID NOs: 6, GAS, and 9, respectively, and specifically binds to a SARS-CoV-2 spike protein, and neutralizes a SARS-CoV-2.

In some embodiments, the antibody or antigen binding fragment comprises a V_{H} comprising a HCDR1, a HCDR2, and a HCDR3 as set forth as SEQ ID NOs: 2, 91, and 47, respectively, a V_{L} comprising a LCDR1, a LCDR2, and a LCDR3 as set forth as SEQ ID NOs: 6, GAS, and 9, respectively, wherein the V_{H} comprises an amino acid sequence at least 90% identical to SEQ ID NO: 46, such as 95%, 96%, 97%, 98% or 99% identical to SEQ ID NO: 46, and wherein the V_{L} comprises an amino acid sequence at least 90% identical to SEQ ID NO: 17, such as 95%, 96%, 97%, 98% or 99% identical to SEQ ID NO: 17, and the antibody or antigens binding fragment specifically binds to a SARS-CoV-2 spike protein, and neutralizes a SARS-CoV-2. In this embodiment, variations due to sequence identify fall outside the CDRs.

In some embodiments, the antibody or antigen binding fragment comprises a V_{H} comprising the amino acid sequence set forth as SEQ ID NO: 46, and specifically binds to a SARS-CoV-2 spike protein, and neutralizes a SARS-CoV-2. In more embodiments, the antibody or antigen binding fragment comprises a V_{L} comprising the amino acid sequence set forth as SEQ ID NO: 17, and specifically binds to a SARS-CoV-2 spike protein, and neutralizes a SARS-CoV-2. In some embodiments, the antibody or antigen binding fragment comprises a V_{H} and a V_{L} comprising the amino acid sequences set forth as SEQ ID NOs: 46 and 17, respectively, and specifically binds to a SARS-CoV-2 spike protein, and neutralizes a SARS-CoV-2.

In some embodiments, the disclosed antibodies inhibit viral entry and/or replication.

### 1. Additional Antibodies

In some examples, antibodies that bind to an epitope of interest can be identified based on their ability to cross-compete (for example, to competitively inhibit the binding of, in a statistically significant manner) with the antibodies provided herein in binding assays. In other examples, antibodies that bind to an epitope of interest can be identified based on their ability to cross-compete (for example, to competitively inhibit the binding of, in a statistically significant manner) with the one or more of the antibodies provided herein in binding assays.

Human antibodies that bind to the same epitope on the spike of the coronavirus protein, such as the spike protein of a SARS CoV-2 BA.4 variant or a BA.5 variant, to which the disclosed antibodies bind can be produced using any suitable method. Such antibodies may be prepared, for example, by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Additional human antibodies that bind to the same epitope can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology. 20 (3): 927-937 (2005) and Vollmers and Brandlein, Methods and Findings in Experimental and Clinical Pharmacology. 27 (3): 185-91 (2005). Human antibodies may also be generated by isolating Fv clone variable domain sequences selected from human-derived phage display libraries. Such variable domain sequences may then be combined with a desired human constant domain.

Antibodies and antigen binding fragments that specifically bind to the same epitope can also be isolated by screening combinatorial libraries for antibodies with the desired binding characteristics. For example, by generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352:624-628 (1991); Marks et al., J. Mol. Biol. 222:581-597 (1992); Marks and Bradbury, in Methods in Molecular Biology 248:161-175 (Lo, ed., Human Press, Totowa, N.J., 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J. 12:725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Competitive binding assays, similar to those disclosed in the examples section below, can be used to select antibodies with the desired binding characteristics.

### 2. Additional Description of Antibodies and Antigen Binding Fragments

An antibody or antigen binding fragment of the antibodies disclosed herein can be a human antibody or fragment thereof. Chimeric antibodies are also provided. The antibody or antigen binding fragment can include any suitable framework region, such as (but not limited to) a human framework region from another source, or an optimized framework region. Alternatively, a heterologous framework region, such as, but not limited to a mouse or monkey framework region, can be included in the heavy or light chain of the antibodies.

The antibody can be of any isotype. The antibody can be, for example, an IgA. IgM or an IgG antibody, such as IgG₁, IgG₂, IgG₃, or IgG₄. The class of an antibody that specifically binds to a coronavirus spike protein can be switched with another. In one aspect, a nucleic acid molecule encoding V_{L} or V_{H} is isolated such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved, for example, using a vector or nucleic acid molecule that comprises a C_{L} or C_{H} chain. For example, an antibody that specifically binds the spike protein, that was originally IgG may be class switched to an IgA. Class switching can be used to convert one IgG subclass to another, such as from IgG₁ to IgG₂, IgG₃, or IgG₄.

In some examples, the disclosed antibodies arc oligomers of antibodies, such as dimers, trimers, tetramers, pentamers, hexamers, septamers, octomers and so on.

The antibody or antigen binding fragment can be derivatized or linked to another molecule (such as another peptide or protein). In general, the antibody or antigen binding fragment is derivatized such that the binding to the spike protein is not affected adversely by the derivatization or labeling. For example, the antibody or antigen binding fragment can be functionally linked (by chemical coupling, genetic fusion, noncovalent association or otherwise) to one or more other molecular entities, such as another antibody (for example, a bi-specific antibody or a diabody), a detectable marker, an effector molecule, or a protein or peptide that can mediate association of the antibody or antibody portion with another molecule (such as a streptavidin core region or a polyhistidine tag).

### (a) Binding Affinity

In several embodiments, the antibody or antigen binding fragment specifically binds the SARS-CoV-2 spike protein with an affinity (e.g., measured by K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. In some embodiment, the SARS-CoV-2 is the BA.4 or the BA.5 variant.

K_{D} can be measured, for example, by a radiolabeled antigen binding assay (RIA) performed with the Fab version of an antibody of interest and its antigen. In one assay, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293 (4): 865-881, 1999). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (NUNC™ Catalog #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57 (20): 4593-4599, 1997). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT™-20; PerkinElmer) is added, and the plates are counted on a TOPCOUNT™ gamma counter (PerkinElmer) for ten minutes. Concentrations of each Fab that give less than or equal to 20% of maximal binding are chosen for use in competitive binding assays.

In another assay, K_{D} can be measured using surface plasmon resonance assays using Biolayer interferometry (BLI), see the examples section. In other embodiments, K_{D} can be measured using a BIACORE®-2000 or a BIACORE®-3000 (BIAcorc, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Affinity can also be measured by high throughput SPR using the Carterra LSA.

### (b) Multispecific Antibodies

In some embodiments, a multi-specific antibody, such as a bi-specific antibody, is provided that comprises an antibody or antigen binding fragment that specifically binds a coronavirus spike protein, as provided herein. Any suitable method can be used to design and produce the multi-specific antibody, such as crosslinking two or more antibodies, antigen binding fragments (such as scFvs) of the same type or of different types. Exemplary methods of making multispecific antibodies include those described in PCT Pub. No. WO2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

The multi-specific antibody may have any suitable format that allows for binding to the coronavirus spike protein by the antibody or antigen binding fragment as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158 (8): 3965-3970, 1997; Mack et al., Proc. Natl. Acad. Sci. U.S.A., 92 (15): 7021-7025, 1995; Kufer et al., Cancer Immunol. Immunother., 45 (3-4): 193-197, 1997; Löffler et al., Blood, 95 (6): 2098-2103, 2000; and Brühl et al., J. Immunol., 166 (4): 2420-2426, 2001. Production of bispecific Fab-scFv (“bibody”) molecules are described, for example, in Schoonjans et al. (J. Immunol., 165 (12): 7050-7057, 2000) and Willems et al. (J. Chromatogr. B Analyt. Technol. Biomed Life Sci. 786 (1-2): 161-176, 2003). For bibodies, a scFv molecule can be fused to one of the V_{L}-C_{L} (L) or V_{H}-CH1 chains, e.g., to produce a bibody one scFv is fused to the C-term of a Fab chain.

The bispecific tetravalent immunoglobulin known as the dual variable domain immunoglobulin or DVD-immunoglobulin molecule is disclosed in Wu et al., MAbs. 2009; 1:339-47, doi: 10.4161/mabs.1.4.8755, incorporated herein by reference. See also Nat Biotechnol. 2007 November; 25 (11): 1290-7. doi: 10.1038/nbt1345. Epub 2007 Oct. 14., also incorporated herein by reference. A DVD-immunoglobulin molecule includes two heavy chains and two light chains. Unlike IgG, however, both heavy and light chains of a DVD-immunoglobulin molecule contain an additional variable domain (VD) connected via a linker sequence at the N-termini of the VH and VL of an existing monoclonal antibody (mAb). Thus, when the heavy and the light chains combine, the resulting DVD-immunoglobulin molecule contains four antigen recognition sites, see Jakob et al., Mabs 5:358-363, 2013, incorporated herein by reference, see FIG. 1 of Jakob et al. for schematic and space-filling diagrams. A DVD-immunoglobulin molecule functions to bind two different antigens on each DFab simultaneously.

The outermost or N-terminal variable domain is termed VD1 and the innermost variable domain is termed VD2; the VD2 is proximal to the C-terminal CH1 or CL. As disclosed in Jakob et al., supra, DVD-immunoglobulin molecules can be manufactured and purified to homogeneity in large quantities, have pharmacological properties similar to those of a conventional IgG₁, and show in vivo efficacy. Any of the disclosed monoclonal antibodies can be included in a DVD-immunoglobulin format.

### (c) Antigen Binding Fragments

Antigen binding fragments are encompassed by the present disclosure, such as Fab, F(ab′)₂, and Fv which include a heavy chain and V_{L} and specifically bind a coronavirus spike protein. These antibody fragments retain the ability to selectively bind with the antigen and are “antigen-binding” fragments. Non-limiting examples of such fragments include:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain;
(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;
(4) Fv, a genetically engineered fragment containing the V_{L} and V_{L} expressed as two chains; and
(5) Single chain antibody (such as scFv), defined as a genetically engineered molecule containing the V_{H} and the V_{L} linked by a suitable polypeptide linker as a genetically fused single chain molecule (see, e.g., Ahmad et al., Clin. Dev. Immunol., 2012, doi: 10.1155/2012/980250; Marbry and Snavely, IDrugs, 13 (8): 543-549, 2010). The intramolecular orientation of the V_{H}-domain and the V_{L}-domain in a scFv, is not decisive for the provided antibodies (e.g., for the provided multispecific antibodies). Thus, scFvs with both possible arrangements (V_{H}-domain-linker domain-V_{L}-domain; V_{L}-domain-linker domain-V_{H}-domain) may be used.
(6) A dimer of a single chain antibody (scFV₂), defined as a dimer of a scFV. This has also been termed a “miniantibody.”

Any suitable method of producing the above-discussed antigen binding fragments may be used. Non-limiting examples are provided in Harlow and Lane, Antibodies: A Laboratory Manual, 2^{nd}, Cold Spring Harbor Laboratory, New York, 2013.

Antigen binding fragments can be prepared by proteolytic hydrolysis of the antibody or by expression in a host cell (such as an E. coli cell) of DNA encoding the fragment. Antigen binding fragments can also be obtained by pepsin or papain digestion of whole antibodies by conventional methods. For example, antigen binding fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

### (d) Variants

In some embodiments, amino acid sequence variants of the antibodies provided herein are provided. For example, it may be desirable to improve the binding affinity and/or other biological properties of the antibody. Amino acid sequence variants of an antibody may be prepared by introducing appropriate modifications into the nucleotide sequence encoding the antibody V_{H} domain and/or V_{L} domain, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some embodiments, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pl and low toxicity of the molecules. Amino acid substitutions can be made in the V_{H} and the V_{L} regions to increase yield.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 9.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 12.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 14.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 15. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 13.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 15. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 18. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 22. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 24. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 26. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 28. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 30. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 32. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 34. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 36. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 38. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 40. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 42. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 44. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 46. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 20.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 50.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 52.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 54.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 62.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 64.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 1. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 66.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 46. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 5.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 46. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 8.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 68. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 70.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 15. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 70.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 72.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 73.

In some embodiments, the Vu of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 74.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 76.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 77.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 79.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 81.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 82.

In some embodiments, the Vu of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 83.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 85.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 10. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 89.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 15. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 77.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 15. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 12.

In some embodiments, the V_{H} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 46. In some embodiments, the V_{L} of the antibody comprises up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as one of SEQ ID NO: 17.

In some embodiments, the antibody or antigen binding fragment can include up to 10 (such as up to 1, up to 2, up to 3, up to 4, up to 5, up to 6, up to 7, up to 8, or up to 9) amino acid substitutions (such as conservative amino acid substitutions) in the framework regions of the heavy chain of the antibody/bispecific antibody, or the light chain of the antibody, or the heavy and light chains of the antibody, compared to known framework regions, or compared to the framework regions of the antibody, and maintain the specific binding activity for the epitope of the spike protein.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant V_{H} and V_{L} sequences provided above, each CDR either is unaltered, or contains no more than one, two or three amino acid substitutions. In some embodiments of the variant V_{H} and V_{L} sequences provided above, only the framework residues are modified so the CDRs are unchanged. Thus, any variation is in the framework regions only.

To increase binding affinity of the antibody, the V_{L} and V_{H} segments can be randomly mutated, such as within HCDR3 region or the LCDR3 region, in a process analogous to the in vivo somatic mutation process responsible for affinity maturation of antibodies during a natural immune response. Thus in vitro affinity maturation can be accomplished by amplifying V_{H} and V_{L} regions using PCR primers complementary to the HCDR3 or LCDR3, respectively. In this process, the primers have been “spiked” with a random mixture of the four nucleotide bases at certain positions such that the resultant PCR products encode V_{H} and V_{L} segments into which random mutations have been introduced into the V_{H} and/or V_{L} CDR3 regions. These randomly mutated V_{H} and V_{L} segments can be tested to determine the binding affinity for the spike protein. In particular examples, the V_{H} amino acid sequence is one of SEQ ID NOs: 1, 10, 15, 18, 21, 24, 26, 28, 30, 32, 34, 36, 38, 40, 42, 44, or 46. In other examples, the V_{L} amino acid sequence is one of SEQ ID NOs: 5, 8, 12, 13, 14, 17, 20, 50, 52, 54, 58, 62, 64, 66, 70, 72, 73, 74, 76, 77, 79, 81, 82, 83, 85, and 89., respectively,

In some embodiments, an antibody or antigen binding fragment is altered to increase or decrease the extent to which the antibody or antigen binding fragment is glycosylated. Addition or deletion of glycosylation sites may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15 (1): 26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In one embodiment, variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336 (5): 1239-1249, 2004; Yamane-Ohnuki et al., Biotechnol. Bioeng. 87 (5): 614-622, 2004. Examples of cell lines capable of producing defucosylated antibodies include Lec 13 CHO cells deficient in protein fucosylation (Ripka et al., Arch. Biochem. Biophys. 249 (2): 533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87 (5): 614-622, 2004; Kanda et al., Biotechnol. Bioeng., 94 (4): 680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several embodiments, the constant region of the antibody or bispecific antibody comprises one or more amino acid substitutions to optimize in vivo half-life of the antibody. The serum half-life of IgG Abs is regulated by the neonatal Fc receptor (FcRn). Thus, in several embodiments, the antibody comprises an amino acid substitution that increases binding to the FcRn. Non-limiting examples of such substitutions include substitutions at IgG constant regions T250Q and M428L (see, e.g., Hinton et al., J Immunol., 176 (1): 346-356, 2006); M428L and N434S (the “LS” mutation, see, e.g., Zalevsky, et al., Nature Biotechnol., 28 (2): 157-159, 2010); N434A (see, e.g., Petkova et al., Int. Immunol., 18 (12): 1759-1769, 2006); T307A, E380A, and N434A (see, e.g., Petkova et al., Int. Immunol., 18 (12): 1759-1769, 2006); and M252Y, S254T, and T256E (see, e.g., Dall'Acqua et al., J. Biol. Chem., 281 (33): 23514-23524, 2006). The disclosed antibodies and antigen binding fragments can be linked to or comprise an Fc polypeptide including any of the substitutions listed above, for example, the Fc polypeptide can include the M428L and N434S substitutions according to the EU index numbering system.

In some embodiments, an antibody or bispecific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Conjugates

The antibodies, antigen binding fragments, and bispecific antibodies that specifically bind to a coronavirus spike protein, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ¹²⁵I, ³²P, ¹⁴C, ³H and ³⁵S and other labels, target moieties, enzymes and ligands, etc. The choice of a particular effector molecule or detectable marker depends on the particular target molecule or cell, and the desired biological effect.

The procedure for attaching a detectable marker to an antibody, antigen binding fragment, or bispecific antibody, varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody, antigen binding fragment, or bispecific antibody, is derivatized to expose or attach additional reactive functional groups. The derivatization may involve attachment of any suitable linker molecule. The linker is capable of forming covalent bonds to both the antibody or antigen binding fragment and to the effector molecule or detectable marker. Suitable linkers include, but are not limited to, straight or branched-chain carbon linkers, heterocyclic carbon linkers, or peptide linkers. Where the antibody, antigen binding fragment, or bispecific antibody, and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to an antibody or antigen binding fragment or bispecific antibody can be determined.

The antibody, antigen binding fragment or bispecific antibody can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). An antibody, antigen binding fragment, or bispecific antibody, can also be conjugated with enzymes that are useful for detection, such as horseradish peroxidase, β-galactosidase, luciferase, alkaline phosphatase, glucose oxidase and the like. When an antibody or antigen binding fragment is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. An antibody, antigen binding fragment, or bispecific antibody, may also be conjugated with biotin, and detected through indirect measurement of avidin or streptavidin binding. It should be noted that the avidin itself can be conjugated with an enzyme or a fluorescent label.

The antibody, antigen binding fragment or bispecific antibody, can be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody, antigen binding fragment, or bispecific antibody, may also be labeled with a predetermined polypeptide epitope recognized by a secondary reporter (such as leucine zipper pair sequences, binding sites for secondary antibodies, metal binding domains, epitope tags).

The antibody, antigen binding fragment or bispecific antibody, can also be conjugated with a radiolabeled amino acid, for example, for diagnostic purposes. For instance, the radiolabel may be used to detect a coronavirus by radiography, emission spectra, or other diagnostic techniques. Examples of labels for polypeptides include, but are not limited to, the following radioisotopes: ³H, ¹⁴C, ³⁵S, ⁹Y, ⁹⁹mTc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody, antigen binding fragment, or bispecific antibody in a conjugate can range, for example, from 1 to 20 moieties per antibody or antigen binding fragment. In some embodiments, the average number of effector molecules or detectable marker moieties per antibody or antigen binding fragment in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, (iv) engineering by recombinant techniques the amino acid sequence of the antibody such that the number and position of cysteine residues is modified for control of the number or position of linker-effector molecule attachments.

### C. Polynucleotides and Expression

Nucleic acid molecules (for example, cDNA or RNA molecules, such as mRNA) encoding the amino acid sequences of antibodies, antigen binding fragments, bispecific antibodies, and conjugates that specifically bind to a coronavirus spike protein, as disclosed herein, are provided. Nucleic acids encoding these molecules can readily be produced using the amino acid sequences provided herein (such as the CDR sequences and V_{H} and V_{L} sequences), sequences available in the art (such as framework or constant region sequences), and the genetic code. In several embodiments, nucleic acid molecules can encode the V_{H}, the V_{L}, or both the V_{H} and V_{L} (for example in a bicistronic expression vector) of a disclosed antibody or antigen binding fragment. In some embodiments, the nucleic acid molecules encode an scFv. In several embodiments, the nucleic acid molecules can be expressed in a host cell (such as a mammalian cell) to produce a disclosed antibody or antigen binding fragment. Nucleic acid molecules encoding an scFv are provided.

The genetic code can be used to construct a variety of functionally equivalent nucleic acid sequences, such as nucleic acids which differ in sequence but which encode the same antibody sequence, or encode a conjugate or fusion protein including the V_{L} and/or V_{H} nucleic acid sequence. In a non-limiting example, an isolated nucleic acid molecule encodes the V_{H} of the B1-182.1HC/182.1LC_94GLTG, B1-182.1HC/182.1LC_93VGLTG, A23-58.1HC/58.1LC_94GLTG. A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.ILC, B1-182.1HC_GStoVV/182.1LC, B1-182.1HC_S100aV/182.ILC, B1-182.1HC_F100cY/182.1LC, B1-182.1HC_Y96N/182.1LC, B1-182.1HC_G-StoN-V/182.1LC, B1-182.1HC_YSFtoNVY/182.1LC, B1-182.1HC_Y-StoN-V/182.1LC, B1-182.1HC_GGtoNV/182.1LC, B1-182.1HC_YGGtoNNV/182.1LC, B1-182.1HC_-GSFto-VVY/182.1LC, B1-182.1HC_G100V/182.ILC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100cS/182.1LC_94GLTG, B1-182.1HC_F100cS/182.1LC_93VGLTG, A23-58.1HC_Y100cS/58.1LC, A23-58.1HC_Y100cA/58.1LC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.1LC_95LTG, A23-58.1HC/58.1LC_96TG, A23-58.1HC/58.1LC_F88Y-94GLTG, A23-58.1HC_Y100cA/58.1LC_S95L, A23-58.1HC_Y100cA/58.1LC_94GLTG, or B1-182.1HC_F100cS/S2E12LC antibody. In another non-limiting example, the nucleic acid molecule encodes the V_{L} of the B1-182.1HC/182.1LC_94GLTG, B1-182.1HC/182.1LC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.ILC_93VGLTG, A23-58.1HC/S2E12LC, A23-58.1HC_Y100cA/S2E12LC, B1-182.1HC/S2E12LC, B1-182.1HC_GGStoNVV/182.ILC, B1-182.1HC_GStoVV/182.1LC, B1-182.1HC_S100aV/182.ILC, B1-182.1HC_F100cY/182.1LC, B1-182.1HC_Y96N/182.1LC, B1-182.1HC_G-StoN-V/182.1LC, B1-182.1HC_YSFtoNVY/182.1LC, B1-182.1HC_Y-StoN-V/182.1LC, B1-182.1HC_GGtoNV/182.1LC, B1-182.1HC_YGGtoNNV/182.1LC, B1-182.1HC_-GSFto-VVY/182.1LC, B1-182.1HC_G100V/182.ILC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100cS/182.1LC_94GLTG, B1-182.1HC_F100cS/182.1LC_93VGLTG, A23-58.1HC_Y100cS/58.1LC, A23-58.1HC_Y100cA/58.ILC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.1LC_95LTG, A23-58.1HC/58.1LC_96TG, A23-58.1HC/58.1LC_F88Y-94GLTG, A23-58.1HC_Y100cA/58.1LC_S95L, A23-58.1HC_Y100cA/58.1LC_94GLTG, or B1-182.1HC_F100cS/S2E12LC antibody. In another non-limiting example, the nucleic acid molecule encodes both the V_{H} and V_{L} of the B1-182.1HC/182.1LC_94GLTG, B1-182.1HC/182.1LC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.1LC, B1-182.1HC_GStoVV/182.1LC, B1-182.1HC_S100aV/182.ILC, B1-182.1HC_F100cY/182.1LC, B1-182.1HC_Y96N/182.1LC, B1-182.1HC_G-StoN-V/182.ILC, B1-182.1HC_YSFtoNVY/182.ILC, B1-182.1HC_Y-StoN-V/182.1LC, B1-182.1HC_GGtoNV/182.1LC, B1-182.1HC_YGGtoNNV/182.1LC, B1-182.1HC_-GSFto-VVY/182.1LC, B1-182.1HC_G100V/182.ILC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100cS/182.1LC_94GLTG, B1-182.1HC_F100cS/182.1LC_93VGLTG, A23-58.1HC_Y100cS/58.1LC, A23-58.1HC_Y100cA/58.1LC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.ILC_95LTG, A23-58.1HC/58.1LC_96TG, A23-58.1HC/58.1LC_F88Y-94GLTG, A23-58.1HC_Y100cA/58.1LC_S95L, A23-58.1HC_Y100cA/58.1LC_94GLTG, or B1-182.1HC_F100cS/S2E12LC antibody. In further non-limiting examples, the nucleic acid molecule can encode a bi-specific antibody, such as in DVD-immunoglobulin format. The nucleic acid can also encode an scFv. The nucleic acid molecule can also encode a conjugate.

Nucleic acid molecules encoding the antibodies, antigen binding fragments, bispecific antibodies, and conjugates that specifically bind to a coronavirus spike protein can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The antibodies, antigen binding fragments, and conjugates can be expressed as individual proteins including the V_{H} and/or V_{L} (linked to an effector molecule or detectable marker as needed), or can be expressed as a fusion protein. Any suitable method of expressing and purifying antibodies and antigen binding fragments may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and V_{L}, and immunoadhesin are provided. The nucleic acid sequences can optionally encode a leader sequence.

To create a scFv the V_{H}- and V_{L}-encoding DNA fragments can be operatively linked to another fragment encoding a flexible linker, e.g., encoding the amino acid sequence (Gly4-Ser) 3, such that the V_{H} and V_{L} sequences can be expressed as a contiguous single-chain protein, with the V_{L} and V_{H} domains joined by the flexible linker (see, e.g., Bird et al., Science, 242 (4877): 423-426, 1988; Huston et al., Proc. Natl. Acad. Sci. U.S.A., 85 (16): 5879-5883, 1988; McCafferty et al., Nature, 348:552-554, 1990; Kontermann and Dübel (Eds.), Antibody Engineering, Vols. 1-2, 2^{nd} ed., Springer-Verlag, 2010; Greenfield (Ed.), Antibodies: A Laboratory Manual, 2^{nd} ed. New York: Cold Spring Harbor Laboratory Press, 2014). Optionally, a cleavage site can be included in a linker, such as a furin cleavage site.

The single chain antibody may be monovalent, if only a single V_{H} and V_{L} are used, bivalent, if two V_{H} and V_{L} are used, or polyvalent, if more than two V_{H} and V_{L} are used. Bispecific or polyvalent antibodies may be generated that bind specifically to a coronavirus spike protein and another antigen. The encoded V_{H} and V_{L} optionally can include a furin cleavage site between the V_{H} and V_{L} domains. Linkers can also be encoded, such as when the nucleic acid molecule encodes a bi-specific antibody in DVD-Ig™ format.

One or more DNA sequences encoding the antibodies, antigen binding fragments, bispecific antibodies, or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed antibodies and antigen binding fragments. Methods of stable transfer, meaning that the foreign DNA is continuously maintained in the host may be used. Hybridomas expressing the antibodies of interest are also encompassed by this disclosure.

The expression of nucleic acids encoding the antibodies, antigen binding fragments, and bispecific antibodies (such as DVD-immunoglobulin antibodies) described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the antibodies, antigen binding fragments, bispecific antibodies, and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The antibodies, antigen binding fragment, and conjugates need not be 100% pure. Once purified, partially or to homogeneity as desired, if to be used prophylatically, the polypeptides should be substantially free of endotoxin.

Methods for expression of antibodies, antigen binding fragments, bispecific antibodies, and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341 (6242): 544-546, 1989.

### D. Methods and Compositions

### 1. Inhibiting a SARS-CoV-2 Infection

Methods are disclosed herein for the inhibition of a coronavirus infection in a subject, such as a SARS-CoV-2 infection. In some embodiments, the SARS-CoV-2 is a BA.4 variant or a BA.5 variant. The methods include administering to the subject an effective amount (that is, an amount effective to inhibit the infection in the subject) of a disclosed antibody, antigen binding fragment, or bispecific antibody, or a nucleic acid encoding such an antibody, antigen binding fragment, or bispecific antibody, to a subject at risk of a coronavirus infection or having the coronavirus infection. The methods can be used pre-exposure or post-exposure. In some embodiments, the antibody or antigen binding fragment can be used in the form of a bi-specific antibody, such as a DVD-Immunoglobulin. The antigen binding fragment can be an scFv.

The infection does not need to be completely eliminated or inhibited for the method to be effective. For example, the method can decrease the infection by a desired amount, for example by at least 10%, at least 20%, at least 50%, at least 60%, at least 70%, at least 80%, at least 90%, at least 95%, at least 98%, or even at least 100% (elimination or prevention of detectable coronavirus infection) as compared to the SARS-CoV-2 infection in the absence of the treatment. In some embodiments, the subject can also be treated with an effective amount of an additional agent, such as an anti-viral agent.

In some embodiments, administration of an effective amount of a disclosed antibody, antigen binding fragment, bispecific antibody, or nucleic acid molecule, inhibits the establishment of an infection and/or subsequent disease progression in a subject, which can encompass any statistically significant reduction in activity (for example, growth or invasion) or symptoms of the coronavirus infection in the subject.

Methods are disclosed herein for the inhibition of a SARS-CoV-2 replication in a subject. The methods include administering to the subject an effective amount (that is, an amount effective to inhibit replication in the subject) of a disclosed antibody, antigen binding fragment, bispecific antibody, or a nucleic acid encoding such an antibody, antigen binding fragment, or bispecific antibody, to a subject at risk of a SARS-CoV-2 infection or having a coronavirus infection. The methods can be used pre-exposure or post-exposure.

Methods are disclosed for treating a SARS-CoV-2 infection in a subject. Methods are also disclosed for preventing a coronavirus infection in a subject. These methods include administering one or more of the disclosed antibodies, antigen binding fragments, bispecific antibodies, or nucleic acid molecule encoding such molecules, or a composition including such molecules, as disclosed herein.

Antibodies, antigen binding fragments thereof, and bispecific antibodies can be administered by intravenous infusion. Doses of the antibody, antigen binding fragment, or bispecific antibody vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some embodiments, the dose of the antibody, antigen binding fragment or bispecific antibody can be from about 0.5 mg/kg to about 5 mg/kg, such as a dose of about 1 mg/kg, about 2 mg/kg, about 3 mg/kg, about 4 mg/kg or about 5 mg/kg. The antibody, antigen binding fragment, or bispecific antibody is administered according to a dosing schedule determined by a medical practitioner. In some examples, the antibody, antigen binding fragment or bispecific antibody is administered weekly, every two weeks, every three weeks or every four weeks.

In some embodiments, the method of inhibiting the infection in a subject further comprises administration of one or more additional agents to the subject. Additional agents of interest include, but are not limited to, anti-viral agents such as hydroxychloroquine, arbidol, remdesivir, favipiravir, baricitinib, lopinavir/ritonavir, Zinc ions, and interferon beta-1b, or their combinations. In some embodiments, the method comprises administration of a first antibody that specifically binds to a coronavirus spike protein as disclosed herein and a second antibody that also specifically binds to a coronavirus protein, such as a different epitope of the coronavirus protein. In some embodiments, the first antibody is one of B1-182.1HC/182.1LC_94GLTG, B1-182.1HC/182.ILC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.ILC, B1-182.1HC_GStoVV/182.ILC, B1-182.1HC_S100aV/182.1LC, B1-182.1HC_F100cY/182.1LC, B1-182.1HC_Y96N/182.1LC, B1-182.1HC_G-StoN-V/182.1LC, B1-182.1HC_YSFtoNVY/182.ILC, B1-182.1HC_Y-StoN-V/182.1LC, B1-182.1HC_GGtoNV/182.1LC, B1-182.1HC_YGGtoNNV/182.1LC, B1-182.1HC_-GSFto-VVY/182.1LC, B1-182.1HC_G100V/182.1LC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100S/182.1LC_94GLTG, B1-182.1HC_F100cS/182.1LC_93VGLTG, A23-58.1HC_Y100cS/58.1LC, A23-58.1HC_Y100cA/58.1LC, A23-58.1HC/58.ILC_S51G, A23-58.1HC/58.ILC_F88Y, A23-58.1HC/58.ILC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.1LC_95LTG, A23-58.1HC/58.1LC_96TG, A23-58.1HC/58.1LC_F88Y-94GLTG, A23-58.1HC_Y100cA/58.1LC_S95L, A23-58.1HC_Y100cA/58.ILC_94GLTG, B1-182.1HC_F100cS/S2E12LC. In more embodiments, the first antibody is one of B1-182.1HC/182.1LC_94GLTG, B1-182.1HC/182.1LC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.ILC, B1-182.1HC_GStoVV/182.1LC, B1-182.1HC_S100aV/182.1LC, B1-182.1HC_F100cY/182.ILC, B1-182.1HC_Y96N/182.1LC, B1-182.1HC_G-StoN-V/182.1LC, B1-182.1HC_YSFtoNVY/182.1LC, B1-182.1HC_Y-StoN-V/182.1LC, B1-182.1HC_GGtoNV/182.ILC, B1-182.1HC_YGGtoNNV/182.ILC, B1-182.1HC_-GSFto-VVY/182.ILC, B1-182.1HC_G100V/182.1LC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100S/182.1LC_94GLTG, B1-182.1HC_F100cS/182.1LC_93VGLTG, A23-58.1HC_Y100cS/58.ILC, A23-58.1HC_Y100cA/58.1LC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.1LC_95LTG, A23-58.1HC/58.1LC_96TG, A23-58.1HC/58.1LC_F88Y-94GLTG, A23-58.1HC_Y100cA/58.ILC_S95L, A23-58.1HC_Y100cA/58.1LC_94GLTG, B1-182.1HC_F100cS/S2E12LC, and the second antibody is another of B1-182.1HC/182.1LC_94GLTG, B1-182.1HC/182.1LC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.ILC, B1-182.1HC_GStoVV/182.1LC, B1-182.1HC_S100aV/182.1LC, B1-182.1HC_F100cY/182.1LC, B1-182.1HC_Y96N/182.1LC, B1-182.1HC_G-StoN-V/182.1LC, B1-182.1HC_YSFtoNVY/182.1LC, B1-182.1HC_Y-StoN-V/182.1LC, B1-182.1HC_GGtoNV/182.1LC, B1-182.1HC_YGGtoNNV/182.1LC, B1-182.1HC_-GSFto-VVY/182.1LC, B1-182.1HC_G100V/182.1LC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100S/182.1LC_94GLTG, B1-182.1HC_F100cS/182.1LC_93VGLTG, A23-58.1HC_Y100cS/58.ILC, A23-58.1HC_Y100cA/58.1LC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.1LC_95LTG, A23-58.1HC/58.1LC_96TG, A23-58.1HC/58.1LC_F88Y-94GLTG, A23-58.1HC_Y100cA/58.ILC_S95L, A23-58.1HC_Y100cA/58.1LC_94GLTG, B1-182.1HC_F100cS/S2E12LC. In some embodiments, one antibody binds one epitope of the spike protein, and another antibody binds a different epitope of the spike protein. An effective amount of one, two, three or four, five, or six of B1-182.1HC/182.1LC_94GLTG, B1-182.1HC/182.1LC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.1LC, B1-182.1HC_GStoVV/182.1LC, B1-182.1HC_S100aV/182.1LC, B1-182.1HC_F100cY/182.1LC, B1-182.1HC_Y96N/182.ILC, B1-182.1HC_G-StoN-V/182.ILC, B1-182.1HC_YSFtoNVY/182.ILC, B1-182.1HC_Y-StoN-V/182.1LC, B1-182.1HC_GGtoNV/182.1LC, B1-182.1HC_YGGtoNNV/182.1LC, B1-182.1HC_-GSFto-VVY/182.1LC, B1-182.1HC_G100V/182.1LC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.1LC_95LTG, B1-182.1HC/182.1LC_96TG, B1-182.1HC/182.1LC_96+G, B1-182.1HC_F100S/182.1LC_94GLTG, B1-182.1HC_F100cS/182.1LC_93VGLTG, A23-58.1HC_Y100cS/58.ILC, A23-58.1HC_Y100cA/58.1LC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.ILC_95LTG, A23-58.1HC/58.1LC_96TG, A23-58.1HC/58.1LC_F88Y-94GLTG, A23-58.1HC_Y100cA/58.1LC_S95L, A23-58.1HC_Y100cA/58.1LC_94GLTG, B1-182.1HC_F100cS/S2E12LC can be administered to a subject.

In a specific non-limiting example, two antibodies are administered to the subject. In some embodiments, more than two antibodies are administered to the subject. In some non-limiting examples, 3, 4, or 5 antibodies are administered to the subject.

In some embodiments, a subject is administered DNA or RNA encoding a disclosed antibody, antigen binding fragment, or bispecific antibody, to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody, antigen binding fragments thereof, or bispecific antibody can be placed under the control of a promoter to increase expression. The methods include liposomal delivery of the nucleic acids. Such methods can be applied to the production of an antibody, or antigen binding fragments thereof. In some embodiments, a disclosed antibody or antigen binding fragment is expressed in a subject using the pVRC8400 vector (described in Barouch et al., J. Virol., 79 (14), 8828-8834, 2005, which is incorporated by reference herein).

In several embodiments, a subject (such as a human subject at risk of a coronavirus infection or having a coronavirus infection) can be administered an effective amount of an AAV viral vector that comprises one or more nucleic acid molecules encoding a disclosed antibody, antigen binding fragment, or bispecific antibody. The AAV viral vector is designed for expression of the nucleic acid molecules encoding a disclosed antibody, antigen binding fragment, or bispecific antibody, and administration of the effective amount of the AAV viral vector to the subject leads to expression of an effective amount of the antibody, antigen binding fragment, or bispecific antibody in the subject. Non-limiting examples of AAV viral vectors that can be used to express a disclosed antibody, antigen binding fragment, or bispecific antibody in a subject include those provided in Johnson et al., Nat. Med., 15 (8): 901-906, 2009 and Gardner et al., Nature, 519 (7541): 87-91, 2015, each of which is incorporated by reference herein in its entirety.

In one embodiment, a nucleic acid encoding a disclosed antibody, antigen binding fragment, or bispecific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed antibody, antigen binding fragment, or bispecific antibody, conjugate, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit a coronavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The SARS-CoV-2 spike protein-specific antibody, antigen binding fragment, or bispecific antibody or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an embodiment, the antibody, antigen binding fragment, bispecific antibody or nucleic acid molecule encoding such molecules, or a composition including such molecules, is administered by a single subcutaneous, intravenous, intra-arterial, intraperitoneal, intramuscular, intradermal or intrathecal injection once a day. The antibody, antigen binding fragment, bispecific antibody, conjugate, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the antibody, antigen binding fragment, conjugate, or nucleic acid molecule encoding such molecules, or a composition including such molecules, over a pre-determined period. The osmotic pump or mini-pump can be implanted subcutaneously, or near a target site.

### 2. Compositions

Compositions are provided that include one or more of the coronavirus spike protein-specific antibody, antigen binding fragment, conjugate, or nucleic acid molecule encoding such molecules, that are disclosed herein in a pharmaceutically acceptable carrier. In some embodiments, the composition comprises the B1-182.1HC/182.1LC_94GLTG, B1-182.1HC/182.1LC_93VGLTG, A23-58.1HC/58.1LC_94GLTG, A23-58.1HC/58.1LC_93VGLTG, A23-58.1HC/58.1LC_F88Y-93VGLTG, A23-58.1HC_Y100cA/58.1LC_93VGLTG, A23-58.1HC/S2E12LC, A23-58.1HC_Y100cA/S2E12LC, B1-182.1HC/S2E12LC, B1-182.1HC_GGStoNVV/182.ILC, B1-182.1HC_GStoVV/182.1LC, B1-182.1HC_S100aV/182.1LC, B1-182.1HC_F100cY/182.1LC, B1-182.1HC_Y96N/182.1LC, B1-182.1HC_G-StoN-V/182.ILC, B1-182.1HC_YSFtoNVY/182.ILC, B1-182.1HC_Y-StoN-V/182.ILC, B1-182.1HC_GGtoNV/182.1LC, B1-182.1HC_YGGtoNNV/182.1LC, B1-182.1HC_-GSFto-VVY/182.1LC, B1-182.1HC_G100V/182.1LC, B1-182.1HC_G99N/182.1LC, B1-182.1HC_F100cS/182.1LC, B1-182.1HC/182.1LC_G93V, B1-182.1HC/182.1LC_N94G, B1-182.1HC/182.1LC_S95L, B1-182.1HC/182.1LC_94GLT, B1-182.1HC/182.ILC_95LTG, B1-182.1HC/182.1LC_96TG, B1-182.1HC/182.1LC_96+G, B1-182.1HC_F100S/182.1LC_94GLTG, B1-182.1HC_F100cS/182.1LC_93VGLTG, A23-58.1HC_Y100cS/58.ILC, A23-58.1HC_Y100cA/58.1LC, A23-58.1HC/58.1LC_S51G, A23-58.1HC/58.1LC_F88Y, A23-58.1HC/58.1LC_G93V, A23-58.1HC/58.1LC_T94G, A23-58.1HC/58.1LC_S95L, A23-58.1HC/58.1LC_P96T, A23-58.1HC/58.1LC_94GLT, A23-58.1HC/58.1LC_93VGLT, A23-58.1HC/58.1LC_95LTG, A23-58.1HC/58.1LC_96TG, A23-58.1HC/58.1LC_F88Y-94GLTG, A23-58.1HC_Y100cA/58.1LC_S95L, A23-58.1HC_Y100cA/58.1LC_94GLTG, B1-182.1HC_F100cS/S2E12LC antibody disclosed herein, or an antigen binding fragment thereof. In some embodiments, the composition comprises two, three, four or more antibodies that specifically bind a coronavirus spike protein. The compositions are useful, for example, for example, for the inhibition or detection of a coronavirus infection, such as a SARS-CoV-2 infection.

The compositions can be prepared in unit dosage forms, such as in a kit, for administration to a subject. The amount and timing of administration are at the discretion of the administering physician to achieve the desired purposes. The antibody, antigen binding fragment, bispecific antibody, conjugate, or nucleic acid molecule encoding such molecules can be formulated for systemic or local administration. In one example, the, antigen binding fragment, bispecific antibody, conjugate, or nucleic acid molecule encoding such molecules, is formulated for parenteral administration, such as intravenous administration.

In some embodiments, the antibody, antigen binding fragment, bispecific antibody, or conjugate thereof, in the composition is at least 70% (such as at least 75%, at least 80%, at least 85%, at least 90%, at least 95%, at least 96%, at least 97%, at least 98% or at least 99%) pure. In some embodiments, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the antibody, antigen binding fragment, bispecific antibody, conjugate, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of antibody, antigen binding fragment, bispecific antibody, or conjugate per subject per day (or the corresponding dose of a conjugate including the antibody or antigen binding fragment). Any suitable method may be used for preparing administrable compositions; non-limiting examples are provided in such publications as Remington: The Science and Practice of Pharmacy, 22^{nd} ed., London, UK: Pharmaceutical Press, 2013. In some embodiments, the composition can be a liquid formulation including one or more antibodies, antigen binding fragments, or bispecific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Antibodies, an antigen binding fragment thereof, a bispecific antibody, or a nucleic acid encoding such molecules, can be provided in lyophilized form and rehydrated with sterile water before administration, although they are also provided in sterile solutions of known concentration. A solution including the antibody, antigen binding fragment, bispecific antibody, or a nucleic acid encoding such molecules, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997.

Antibodies, antigen binding fragments, conjugates, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

### 3. Methods of Detection and Diagnosis

Methods are also provided for the detection of the presence of a coronavirus spike protein in vitro or in vivo. In some embodiments, the coronavirus is a BA.4 variant and/or a BA.5 variant. In one example, the presence of a coronavirus spike protein is detected in a biological sample from a subject and can be used to identify a subject with an infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an antibody, antigen binding fragment, or bispecific antibody, that specifically binds to a coronavirus spike protein, or conjugate thereof (e.g., a conjugate including a detectable marker) under conditions sufficient to form an immune complex, and detecting the immune complex (e.g., by detecting a detectable marker conjugated to the antibody or antigen binding fragment).

In one embodiment, the antibody, antigen binding fragment or bispecific antibody is directly labeled with a detectable marker. In another embodiment, the antibody (or antigen binding fragment or bispecific antibody) that binds the coronavirus spike protein (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the antibody, antigen binding fragment, bispecific antibody or secondary antibody are known and described above, and include various enzymes, prosthetic groups, fluorescent materials, luminescent materials, magnetic agents and radioactive materials.

In some embodiments, the disclosed antibodies, antigen binding fragments thereof, or bispecific antibodies are used to test vaccines. For example, to test if a vaccine composition including a coronavirus spike protein or fragment thereof assumes a conformation including the epitope of a disclosed antibody. Thus, provided herein is a method for testing a vaccine, wherein the method comprises contacting a sample containing the vaccine, such as a coronavirus spike protein immunogen, with a disclosed antibody, antigen binding fragment, or bispecific antibody, under conditions sufficient for formation of an immune complex, and detecting the immune complex, to detect the vaccine including the epitope of interest in the sample. In one example, the detection of the immune complex in the sample indicates that vaccine component, such as the immunogen assumes a conformation capable of binding the antibody or antigen binding fragment.

### EXAMPLES

### Example 1

### Engineered SARS-CoV-2 Antibodies with Increased Neutralization Breadth Against Strains BA.4 and BA.5

SARS2-B0001-d30-182.1 (B1-182.1) and SARS2.A23-d48-58.1 (A23-58.1) are potent antibodies with broad neutralizing activity against SARS-CoV-2 variants, see PCT Application No. PCT/US2022/015341. B1-182.1 and A23-58.1 have highly similar sequences but slight differences in neutralization potency, with B1-182.1 generally having better neutralizing activity compared to A23-58.1. However, following transient transfection of both antibodies, when compared to B1-182.1, A23-58.1 has a higher yield (i.e., total amount), higher capacity of concentration docs not precipitate (Table 1). Swapping the CDRH3 region from A23-58.1 into B1-182.1 (B1-182.1_58.1CDRH3 heavy/B1-182.1 light) led to higher yields and eliminated precipitation but a swap of 5 mutation from A23-58.1 light chain into the light chain of B1-182.1 (B1-182.1 heavy/B1-182.1 light_5Mut) did not eliminate precipitation or increase yield (Table 1). In addition, making these swaps did not impact neutralization potency against the SARS-CoV-2 variants tested and modestly increased the neutralization potency against the original Omicron variant (also known as BA.1 and B.1.1.529).

TABLE 1
Yield and precipitation of antibodies B1-182.1, A23.58.1, B1-182.1_58.1CDRH3
heavy/B1-182.1 light and B1-182.1 heavy/B1-182.1 light_5Mut

| Antibody | | Heavy Chain | Light Chain | Yield | |
|---|---|---|---|---|---|
| # | Name | Name | Name | (mg/L) | Precipitation |
| 1 | A23-58.1 | A23-58.1 heavy | A23-58.1 light | 203.75 | None |
| 2 | B1-182.1 | B1-182.1 heavy | B1-182.1 light | 92.36 | Observed |
| 3 | B1-182.1_58.1CDRH3 | B1- | B1-182.1 light | 226.86 | None |
| | heavy/B1-182.1 light | 182.1_58.1CDRH3 | | | |
| | | heavy | | | |
| 4 | B1-182.1 heavy/B1-182.1 | B1-182.1 heavy | B1-182.1 | 74.96 | Observed |
| | light_5Mut | | light_5Mut | | |

Fifty additional variations of B1-182.1 and A23-58.1 were created (Table 2), and subsets were identified which did not precipitate and have increased yields. Variations that maintain or improve the potency over the parental B1-182.1 and/or A23-58.1 against SARS-CoV-2 variants, including Omicron and its sublineages were further identified. Multiple antibodies that show increased potency, lack precipitation and neutralize BA.4 and BA.5 were found. The neutralization of BA.4 and BA.5 is a highly unique feature of these antibodies as currently reported antibodies in the same class do not have this activity.

### Methods

BA.4 and BA.5 neutralization was assessed using a single construct referred to as BA.4/5. This is because, in the spike proteins, the only amino acid difference between BA.4 and BA.5 is found in the signal peptide region. Since this region is not found in mature virus particles, monoclonal antibody neutralization against BA.4 and BA.5 can be evaluated using the single BA.4/5 construct.

TABLE 2
Amino acid sequences for B1-182.1 variation indicated, underlined residues
are altered relative to the parental B1-182.1 or A23-58.1 antibody.
Bold indicates CDR regions in heavy or light chain regions

| | Antibody | | |
|---|---|---|---|
| No. | Name | Heavy Chain | Light Chain |
| 1 | A23-58.1 | QMQLVQSGPEVKKPGTSVKVSCKASG | EIVLTQSPGTLSLSPGERATLSCRASQSV |
| | | FTFTSSAVQWVRQARGQRLEWIGWIV | SSSYLAWYQQKPGQAPRLLIYSASSRATG |
| | | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSPWTFGQGTKVEIK |
| | | VCYDGFDIWGQGTMVTVSS (SEQ | (SEQ ID NO: 70) |
| | | ID NO: 10) | |
| 2 | B1-182.1 | QMQLVQSGPEVKKPGTSVKVSCKASG | EIVLTQSPGTLSLSPGERATLSCRASQSV |
| | | FTFTSSAVQWVRQARGQRLEWIGWIV | SSSYLAWYQQKPGQAPRLLIYGASSRATG |
| | | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSPWTFGQGTKVEIR |
| | | SCFDGFDIWGQGTMVTVSS | (SEQ ID NO: 20) |
| | | (SEQ ID NO: 1) | |
| 3 | B1- | QMQLVQSGPEVKKPGTSVKVSCKASG | EIVLTQSPGTLSLSPGERATLSCRASQSV |
| | 182.1_58.1C | FTFTSSAVQWVRQARGQRLEWIGWIV | SSSYLAWYQQKPGQAPRLLIYGASSRATG |
| | DRH3 heavy/ | VGSGNTNYAQKFQERVTITRDMSTST | FPDRFSGSGSGTDFTLTISRLEPEDFAVY |
| | B1-182.1 | AYMELSSLRSEDTAVYYCAAPNCSNV | YCQQYGNSPWTFGQGTKVEIR |
| | light | VCYDGFDIWGQGTMVTVSS | (SEQ ID NO: 20) |
| | | (SEQ ID NO: 10) | |
| 4 | B1-182.1 | QMQLVQSGPEVKKPGTSVKVSCKASG | EIVLTQSPGTLSLSPGERATLSCRASQSV |
| | heavy /B1- | FTFTSSAVQWVRQARGQRLEWIGWIV | SSSYLAWYQQKPGQAPRLLIYSASSRATG |
| | 182.1 | VGSGNTNYAQKFQERVTITRDMSTST | IPDRFSGSGSGTDFTLTISRLEPEDFAVY |
| | light_5Mut | AYMELSSLRSEDTAVYYCAAPYCSGG | FCQQYGTSPWTFGQGTKVEIK |
| | | SCFDGFDIWGQGTMVTVSS | (SEQ ID NO: 70) |
| | | (SEQ ID NO: 1) | |
| 5 | B1- | QMQLVQSGPEVKKPGTSVKVSCKASG | EIVLTQSPGTLSLSPGERATLSCRASQSV |
| | 182.1HC_GGS | FTFTSSAVQWVRQARGQRLEWIGWIV | SSSYLAWYQQKPGQAPRLLIYGASSRATG |
| | toNVV/182.1 | VGSGNTNYAQKFQERVTITRDMSTST | FPDRFSGSGSGTDFTLTISRLEPEDFAVY |
| | LC | AYMELSSLRSEDTAVYYCAAPYCSNV | YCQQYGNSPWTFGQGTKVEIR |
| | | VCFDGFDIWGQGTMVTVSS | (SEQ ID NO: 20) |
| | | (SEQ ID NO: 18) | |
| 6 | B1- | QMQLVQSGPEVKKPGTSVKVSCKASG | EIVLTQSPGTLSLSPGERATLSCRASQSV |
| | 182.1HC_GSt | FTFTSSAVQWVRQARGQRLEWIGWIV | SSSYLAWYQQKPGQAPRLLIYGASSRATG |
| | oVV/182.1LC | VGSGNTNYAQKFQERVTITRDMSTST | FPDRFSGSGSGTDFTLTISRLEPEDFAVY |
| | | AYMELSSLRSEDTAVYYCAAPYCSGV | YCQQYGNSPWTFGQGTKVEIR |
| | | VCFDGFDIWGQGTMVTVSS | (SEQ ID NO: 20) |
| | | (SEQ ID NO: 22) | |
| 7 | B1- | QMQLVQSGPEVKKPGTSVKVSCKASG | EIVLTQSPGTLSLSPGERATLSCRASQSV |
| | 182.1HC_S10 | FTFTSSAVQWVRQARGQRLEWIGWIV | SSSYLAWYQQKPGQAPRLLIYGASSRATG |
| | 0aV/182.1LC | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSPWTFGQGTKVEIR |
| | | VCFDGFDIWGQGTMVTVSS | (SEQ ID NO: 20) |
| | | (SEQ ID NO: 24) | |
| 8 | B1- | QMQLVQSGPEVKKPGTSVKVSCKASG | EIVLTQSPGTLSLSPGERATLSCRASQSV |
| | 182.1HC_F10 | FTFTSSAVQWVRQARGQRLEWIGWIV | SSSYLAWYQQKPGQAPRLLIYGASSRATG |
| | 0cY/182.1LC | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSPWTFGQGTKVEIR |
| | | SCYDGFDIWGQGTMVTVSS | (SEQ ID NO: 20) |
| | | (SEQ ID NO: 26) | |
| 9 | B1- | QMQLVQSGPEVKKPGTSVKVSCKASG | EIVLTQSPGTLSLSPGERATLSCRASQSV |
| | 182.1HC_Y96 | FTFTSSAVQWVRQARGQRLEWIGWIV | SSSYLAWYQQKPGQAPRLLIYGASSRATG |
| | N/182.1LC | VGSGNTNYAQKFQERVTITRDMSTST | FPDRFSGSGSGTDFTLTISRLEPEDFAVY |
| | | AYMELSSLRSEDTAVYYCAAPNCSGG | YCQQYGNSPWTFGQGTKVEIR |
| | | SCFDGFDIWGQGTMVTVSS | (SEQ ID NO: 20) |
| | | (SEQ ID NO: 28) | |
| 10 | B1- | QMQLVQSGPEVKKPGTSVKVSCKASG | EIVLTQSPGTLSLSPGERATLSCRASQSV |
| | 182.1HC_G- | FTFTSSAVQWVRQARGQRLEWIGWIV | SSSYLAWYQQKPGQAPRLLIYGASSRATG |
| | StoN- | VGSGNTNYAQKFQERVTITRDMSTST | FPDRFSGSGSGTDFTLTISRLEPEDFAVY |
| | V/182.1LC | AYMELSSLRSEDTAVYYCAAPYCSNG | YCQQYGNSPWTFGQGTKVEIR |
| | | VCFDGFDIWGQGTMVTVSS | (SEQ ID NO: 20) |
| | | (SEQ ID NO: 30) | |
| 11 | B1- | QMQLVQSGPEVKKPGTSVKVSCKASG | EIVLTQSPGTLSLSPGERATLSCRASQSV |
| | 182.1HC_YSF | FTFTSSAVQWVRQARGQRLEWIGWIV | SSSYLAWYQQKPGQAPRLLIYGASSRATG |
| | toNVY/182.1 | VGSGNTNYAQKFQERVTITRDMSTST | FPDRFSGSGSGTDFTLTISRLEPEDFAVY |
| | LC | AYMELSSLRSEDTAVYYCAAPNCSGG | YCQQYGNSPWTFGQGTKVEIR |
| | | VCYDGFDIWGQGTMVTVSS | (SEQ ID NO: 20) |
| | | (SEQ ID NO: 32) | |
| 12 | B1- | QMQLVQSGPEVKKPGTSVKVSCKASG | EIVLTQSPGTLSLSPGERATLSCRASQSV |
| | 182 .1HC_Y- | FTFTSSAVQWVRQARGQRLEWIGWIV | SSSYLAWYQQKPGQAPRLLIYGASSRATG |
| | StoN- | VGSGNTNYAQKFQERVTITRDMSTST | FPDRFSGSGSGTDFTLTISRLEPEDFAVY |
| | V/182.1LC | AYMELSSLRSEDTAVYYCAAPNCSGG | YCQQYGNSPWTFGQGTKVEIR |
| | | VCFDGFDIWGQGTMVTVSS | (SEQ ID NO: 20) |
| | | (SEQ ID NO: 34) | |
| 13 | B1- | QMQLVQSGPEVKKPGTSVKVSCKASG | EIVLTQSPGTLSLSPGERATLSCRASQSV |
| | 182.1HC_GGt | FTFTSSAVQWVRQARGQRLEWIGWIV | SSSYLAWYQQKPGQAPRLLIYGASSRATG |
| | ONV/182.1LC | VGSGNTNYAQKFQERVTITRDMSTST | FPDRFSGSGSGTDFTLTISRLEPEDFAVY |
| | | AYMELSSLRSEDTAVYYCAAPYCSNV | YCQQYGNSPWTFGQGTKVEIR |
| | | SCFDGFDIWGQGTMVTVSS | (SEQ ID NO: 20) |
| | | (SEQ ID NO: 36) | |
| 14 | B1- | QMQLVQSGPEVKKPGTSVKVSCKASG | EIVLTQSPGTLSLSPGERATLSCRASQSV |
| | 182.1HC_YGG | FTFTSSAVQWVRQARGQRLEWIGWIV | SSSYLAWYQQKPGQAPRLLIYGASSRATG |
| | toNNV/182.1 | VGSGNTNYAQKFQERVTITRDMSTST | FPDRFSGSGSGTDFTLTISRLEPEDFAVY |
| | LC | AYMELSSLRSEDTAVYYCAAPNCSNV | YCQQYGNSPWTFGQGTKVEIR |
| | | SCFDGFDIWGQGTMVTVSS | (SEQ ID NO: 20) |
| | | (SEQ ID NO: 38) | |
| 15 | B1- | QMQLVQSGPEVKKPGTSVKVSCKASG | EIVLTQSPGTLSLSPGERATLSCRASQSV |
| | 182.1HC_- | FTFTSSAVQWVRQARGQRLEWIGWIV | SSSYLAWYQQKPGQAPRLLIYGASSRATG |
| | GSFto- | VGSGNTNYAQKFQERVTITRDMSTST | FPDRFSGSGSGTDFTLTISRLEPEDFAVY |
| | VVY/182.1LC | AYMELSSLRSEDTAVYYCAAPYCSGV | YCQQYGNSPWTFGQGTKVEIR |
| | | VCYDGFDIWGQGTMVTVSS | (SEQ ID NO: 20) |
| | | (SEQ ID NO: 40) | |
| 16 | B1- | QMQLVQSGPEVKKPGTSVKVSCKASG | EIVLTQSPGTLSLSPGERATLSCRASQSV |
| | 182.1HC_G10 | FTFTSSAVQWVRQARGQRLEWIGWIV | SSSYLAWYQQKPGQAPRLLIYGASSRATG |
| | 0V/182.1LC | VGSGNTNYAQKFQERVTITRDMSTST | FPDRFSGSGSGTDFTLTISRLEPEDFAVY |
| | | AYMELSSLRSEDTAVYYCAAPYCSGV | YCQQYGNSPWTFGQGTKVEIR |
| | | SCFDGFDIWGQGTMVTVSS | (SEQ ID NO: 20) |
| | | (SEQ ID NO: 42) | |
| 17 | B1- | QMQLVQSGPEVKKPGTSVKVSCKASG | EIVLTQSPGTLSLSPGERATLSCRASQSV |
| | 182.1HC_G99 | FTFTSSAVQWVRQARGQRLEWIGWIV | SSSYLAWYQQKPGQAPRLLIYGASSRATG |
| | N/182.1LC | VGSGNTNYAQKFQERVTITRDMSTST | FPDRFSGSGSGTDFTLTISRLEPEDFAVY |
| | | AYMELSSLRSEDTAVYYCAAPYCSNG | YCQQYGNSPWTFGQGTKVEIR |
| | | SCFDGFDIWGQGTMVTVSS | (SEQ ID NO: 20) |
| | | (SEQ ID NO: 44) | |
| 18 | B1- | QMQLVQSGPEVKKPGTSVKVSCKASG | EIVLTQSPGTLSLSPGERATLSCRASQSV |
| | 182.1HC_F10 | FTFTSSAVQWVRQARGQRLEWIGWIV | SSSYLAWYQQKPGQAPRLLIYGASSRATG |
| | 0cs/182.1LC | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSPWTFGQGTKVEIR |
| | | SCSDGFDIWGQGTMVTVSS | (SEQ ID NO: 20) |
| | | (SEQ ID NO: 46) | |
| 19 | B1- | QMQLVQSGPEVKKPGTSVKVSCKASG | EIVLTQSPGTLSLSPGERATLSCRASQSV |
| | 182.1HC_F10 | FTFTSSAVQWVRQARGQRLEWIGWIV | SSSYLAWYQQKPGQAPRLLIYGASSRATG |
| | 0cG/182.1LC | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSPWTFGQGTKVEIR |
| | | SCGDGFDIWGQGTMVTVSS | (SEQ ID NO: 20) |
| | | (SEQ ID NO: 48) | |
| 20 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_F59I | VGSGNTNYAQKFQERVTITRDMSTST | IPDRFSGSGSGTDFTLTISRLEPEDFAVY |
| | | AYMELSSLRSEDTAVYYCAAPYCSGG | YCQQYGNSPWTFGQGTKVEIR |
| | | SCFDGFDIWGQGTMVTVSS | (SEQ ID NO: 93) |
| | | (SEQ ID NO: 1) | |
| 21 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_G93V | VGSGNTNYAQKFQERVTITRDMSTST | FPDRFSGSGSGTDFTLTISRLEPEDFAVY |
| | | AYMELSSLRSEDTAVYYCAAPYCSGG | YCQQYVNSPWTFGQGTKVEIR |
| | | SCFDGFDIWGQGTMVTVSS | (SEQ ID NO: 50) |
| | | (SEQ ID NO: 1) | |
| 22 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_N94G | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGGSPWTFGQGTKVEIR |
| | | SCFDGFDIWGQGTMVTVSS | (SEQ ID NO: 52) |
| | | (SEQ ID NO: 1) | |
| 23 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_S95L | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNLPWTFGQGTKVEIR |
| | | SCFDGFDIWGQGTMVTVSS | (SEQ ID NO: 54) |
| | | (SEQ ID NO: 1) | |
| 24 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_P96T | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSTWTFGQGTKVEIR |
| | | SCFDGFDIWGQGTMVTVSS | (SEQ ID NO: 56) |
| | | (SEQ ID NO: 1) | |
| 25 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_94GLT | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGGLTWTFGQGTKVEIR |
| | | SCFDGFDIWGQGTMVTVSS | (SEQ ID NO: 58) |
| | | (SEQ ID NO: 1) | |
| 26 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_93VGLT | VGSGNTNYAQKFQERVTITRDMSTST | FPDRFSGSGSGTDFTLTISRLEPEDFAVY |
| | | AYMELSSLRSEDTAVYYCAAPYCSGG | YCQQYVGLTWTFGQGTKVEIR |
| | | SCFDGFDIWGQGTMVTVSS | (SEQ ID NO: 60) |
| | | (SEQ ID NO: 1) | |
| 27 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_94GLTG | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGGLTGWTFGQGTKVEIR |
| | | SCFDGFDIWGQGTMVTVSS | (SEQ ID NO: 5) |
| | | (SEQ ID NO: 1) | |
| 28 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_93VGLT | VGSGNTNYAQKFQERVTITRDMSTST | FPDRFSGSGSGTDFTLTISRLEPEDFAVY |
| | G | AYMELSSLRSEDTAVYYCAAPYCSGG | YCQQYVGLTGWTFGQGTKVEIR |
| | | SCFDGFDIWGQGTMVTVSS | (SEQ ID NO: 8) |
| | | (SEQ ID NO: 1) | |
| 29 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_95LTG | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNLTGWTFGQGTKVEIR |
| | | SCFDGFDIWGQGTMVTVSS | (SEQ ID NO: 62) |
| | | (SEQ ID NO: 1) | |
| 30 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_96TG | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSTGWTFGQGTKVEIR |
| | | SCFDGFDIWGQGTMVTVSS | (SEQ ID NO: 64) |
| | | (SEQ ID NO: 1) | |
| 31 | B1- | QMQLVQSGPEVKKPGTSVKVSCKASG | EIVLTQSPGTLSLSPGERATLSCRASQSV |
| | 182.1HC/182 | FTFTSSAVQWVRQARGQRLEWIGWIV | SSSYLAWYQQKPGQAPRLLIYGASSRATG |
| | .1LC_96+G | VGSGNTNYAQKFQERVTITRDMSTST | FPDRFSGSGSGTDFTLTISRLEPEDFAVY |
| | | AYMELSSLRSEDTAVYYCAAPYCSGG | YCQQYGNSPGWTFGQGTKVEIR |
| | | SCFDGFDIWGQGTMVTVSS | (SEQ ID NO: |
| | | (SEQ ID NO: 1) | |
| 32 | B1- | QMQLVQSGPEVKKPGTSVKVSCKASG | EIVLTQSPGTLSLSPGERATLSCRASQSV |
| | 182.1HC_F10 | FTFTSSAVQWVRQARGQRLEWIGWIV | SSSYLAWYQQKPGQAPRLLIYGASSRATG |
| | 0cS/182.1LC | VGSGNTNYAQKFQERVTITRDMSTST | FPDRFSGSGSGTDFTLTISRLEPEDFAVY |
| | _94GLTG | AYMELSSLRSEDTAVYYCAAPYCSGG | YCQQYGGLTGWTFGQGTKVEIR |
| | | SCSDGFDIWGQGTMVTVSS | (SEQ ID NO: 5) |
| | | (SEQ ID NO: 46) | |
| 33 | B1- | QMQLVQSGPEVKKPGTSVKVSCKASG | EIVLTQSPGTLSLSPGERATLSCRASQSV |
| | 182.1HC_F10 | FTFTSSAVQWVRQARGQRLEWIGWIV | SSSYLAWYQQKPGQAPRLLIYGASSRATG |
| | 0cS/182.1LC | VGSGNTNYAQKFQERVTITRDMSTST | FPDRFSGSGSGTDFTLTISRLEPEDFAVY |
| | _93VGLTG | AYMELSSLRSEDTAVYYCAAPYCSGG | YCQQYVGLTGWTFGQGTKVEIR |
| | | SCSDGFDIWGQGTMVTVSS | (SEQ ID NO: 8) |
| | | (SEQ ID NO: 46) | |
| 34 | A23- | QMQLVQSGPEVKKPGTSVKVSCKASG | EIVLTQSPGTLSLSPGERATLSCRASQSV |
| | 58.1HC_Y100 | FTFTSSAVQWVRQARGQRLEWIGWIV | SSSYLAWYQQKPGQAPRLLIYSASSRATG |
| | cS/58.1LC | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSPWTFGQGTKVEIK |
| | | VCSDGFDIWGQGTMVTVSS | (SEQ ID NO: 70) |
| | | (SEQ ID NO: 68) | |
| 35 | A23- | QMQLVQSGPEVKKPGTSVKVSCKASG | EIVLTQSPGTLSLSPGERATLSCRASQSV |
| | 58.1HC_Y100 | FTFTSSAVQWVRQARGQRLEWIGWIV | SSSYLAWYQQKPGQAPRLLIYSASSRATG |
| | CA/58.1LC | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSPWTFGQGTKVEIK |
| | | VCADGFDIWGQGTMVTVSS | (SEQ ID NO: 70) |
| | | (SEQ ID NO: 15) | |
| 36 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYGASSRATG |
| | LC_S51G | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSPWTFGQGTKVEIK |
| | | VCYDGFDIWGQGTMVTVSS | (SEQ ID NO: 72) |
| | | (SEQ ID NO: 10) | |
| 37 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_F88Y | VGSGNTNYAQKFQERVTITRDMSTST | IPDRFSGSGSGTDFTLTISRLEPEDFAVY |
| | | AYMELSSLRSEDTAVYYCAAPNCSNV | YCQQYGTSPWTFGQGTKVEIK |
| | | VCYDGFDIWGQGTMVTVSS | (SEQ ID NO: 73) |
| | | (SEQ ID NO: 10) | |
| 38 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_G93V | VGSGNTNYAQKFQERVTITRDMSTST | IPDRFSGSGSGTDFTLTISRLEPEDFAVY |
| | | AYMELSSLRSEDTAVYYCAAPNCSNV | FCQQYVTSPWTFGQGTKVEIK |
| | | VCYDGFDIWGQGTMVTVSS | (SEQ ID NO: 74) |
| | | (SEQ ID NO: 10) | |
| 39 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_T94G | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGGSPWTFGQGTKVEIK |
| | | VCYDGFDIWGQGTMVTVSS | (SEQ ID NO: 76) |
| | | (SEQ ID NO: 10) | |
| 40 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_S95L | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTLPWTFGQGTKVEIK |
| | | VCYDGFDIWGQGTMVTVSS | (SEQ ID NO: 77) |
| | | (SEQ ID NO: 10) | |
| 41 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_P96T | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSTWTFGQGTKVEIK |
| | | VCYDGFDIWGQGTMVTVSS | (SEQ ID NO: 79) |
| | | (SEQ ID NO: 10) | |
| 42 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_94GLT | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGGLTWTFGQGTKVEIK |
| | | VCYDGFDIWGQGTMVTVSS | (SEQ ID NO: 81) |
| | | (SEQ ID NO: 10) | |
| 43 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_93VGLT | VGSGNTNYAQKFQERVTITRDMSTST | IPDRFSGSGSGTDFTLTISRLEPEDFAVY |
| | | AYMELSSLRSEDTAVYYCAAPNCSNV | FCQQYVGLTWTFGQGTKVEIK |
| | | VCYDGFDIWGQGTMVTVSS | (SEQ ID NO: 82) |
| | | (SEQ ID NO: 10) | |
| 44 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_94GLTG | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGGLTGWTFGQGTKVEIK |
| | | VCYDGFDIWGQGTMVTVSS | (SEQ ID NO: 12) |
| | | (SEQ ID NO: 10) | |
| 45 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_93VGLTG | VGSGNTNYAQKFQERVTITRDMSTST | IPDRFSGSGSGTDFTLTISRLEPEDFAVY |
| | | AYMELSSLRSEDTAVYYCAAPNCSNV | FCQQYVGLTGWTFGQGTKVEIK |
| | | VCYDGFDIWGQGTMVTVSS | (SEQ ID NO: 13) |
| | | (SEQ ID NO: 10) | |
| 46 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_95LTG | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTLTGWTFGQGTKVEIK |
| | | VCYDGFDIWGQGTMVTVSS | (SEQ ID NO: 83) |
| | | (SEQ ID NO: 10) | |
| 47 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_96TG | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSTGWTFGQGTKVEIK |
| | | VCYDGFDIWGQGTMVTVSS | (SEQ ID NO: 85) |
| | | (SEQ ID NO: 10) | |
| 48 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_96+G | VGSGNTNYAQKFQERVTITRDMSTST | IPDRFSGSGSGTDFTLTISRLEPEDFAVY |
| | | AYMELSSLRSEDTAVYYCAAPNCSNV | FCQQYGTSPGWTFGQGTKVEIK |
| | | VCYDGFDIWGQGTMVTVSS | (SEQ ID NO: 87) |
| | | (SEQ ID NO: 10) | |
| 49 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_F88Y- | VGSGNTNYAQKFQERVTITRDMSTST | IPDRFSGSGSGTDFTLTISRLEPEDFAVY |
| | 94GLTG | AYMELSSLRSEDTAVYYCAAPNCSNV | YCQQYGGLTGWTFGQGTKVEIK |
| | | VCYDGFDIWGQGTMVTVSS | (SEQ ID NO: 89) |
| | | (SEQ ID NO: 10) | |
| 50 | A23- | QMQLVQSGPEVKKPGTSVKVSCKASG | EIVLTQSPGTLSLSPGERATLSCRASQSV |
| | 58.1HC/58.1 | FTFTSSAVQWVRQARGQRLEWIGWIV | SSSYLAWYQQKPGQAPRLLIYSASSRATG |
| | LC_F88Y- | VGSGNTNYAQKFQERVTITRDMSTST | IPDRFSGSGSGTDFTLTISRLEPEDFAVY |
| | 93VGLTG | AYMELSSLRSEDTAVYYCAAPNCSNV | YCQQYVGLTGWTFGQGTKVEIK |
| | | VCYDGFDIWGQGTMVTVSS | (SEQ ID NO: 14) |
| | | (SEQ ID NO: 10) | |
| 51 | A23- | QMQLVQSGPEVKKPGTSVKVSCKASG | EIVLTQSPGTLSLSPGERATLSCRASQSV |
| | 58.1HC_Y100 | FTFTSSAVQWVRQARGQRLEWIGWIV | SSSYLAWYQQKPGQAPRLLIYSASSRATG |
| | CA/58.1LC_S | VGSGNTNYAQKFQERVTITRDMSTST | IPDRFSGSGSGTDFTLTISRLEPEDFAVY |
| | 95L | AYMELSSLRSEDTAVYYCAAPNCSNV | FCQQYGTLPWTFGQGTKVEIK |
| | | VCADGFDIWGQGTMVTVSS | (SEQ ID NO: 77) |
| | | (SEQ ID NO: 15) | |
| 52 | 58.1_100cA/ | QMQLVQSGPEVKKPGTSVKVSCKASG | EIVLTQSPGTLSLSPGERATLSCRASQSV |
| | 58.1_93VGLT | FTFTSSAVQWVRQARGQRLEWIGWIV | SSSYLAWYQQKPGQAPRLLIYSASSRATG |
| | | VGSGNTNYAQKFQERVTITRDMSTST | IPDRFSGSGSGTDFTLTISRLEPEDFAVY |
| | | AYMELSSLRSEDTAVYYCAAPNCSNV | FCQQYVGLTWTFGQGTKVEIK |
| | | VCADGFDIWGQGTMVTVSS | (SEQ ID NO: 82) |
| | | (SEQ ID NO: 15) | |
| 53 | A23- | QMQLVQSGPEVKKPGTSVKVSCKASG | EIVLTQSPGTLSLSPGERATLSCRASQSV |
| | 58.1HC_Y100 | FTFTSSAVQWVRQARGQRLEWIGWIV | SSSYLAWYQQKPGQAPRLLIYSASSRATG |
| | cA/58.1LC_9 | VGSGNTNYAQKFQERVTITRDMSTST | IPDRFSGSGSGTDFTLTISRLEPEDFAVY |
| | 4GLTG | AYMELSSLRSEDTAVYYCAAPNCSNV | FCQQYGGLTGWTFGQGTKVEIK |
| | | VCADGFDIWGQGTMVTVSS | (SEQ ID NO: 12) |
| | | (SEQ ID NO: 15) | |
| 54 | A23- | QMQLVQSGPEVKKPGTSVKVSCKASG | EIVLTQSPGTLSLSPGERATLSCRASQSV |
| | 58.1HC_Y100 | FTFTSSAVQWVRQARGQRLEWIGWIV | SSSYLAWYQQKPGQAPRLLIYSASSRATG |
| | cA/58.1LC_9 | VGSGNTNYAQKFQERVTITRDMSTST | IPDRFSGSGSGTDFTLTISRLEPEDFAVY |
| | 3VGLTG | AYMELSSLRSEDTAVYYCAAPNCSNV | FCQQYVGLTGWTFGQGTKVEIK |
| | | VCADGFDIWGQGTMVTVSS | (SEQ ID NO: 13) |
| | | (SEQ ID NO: 15) | |
| 55 | S2E12 | QVQLVQSGPEVKKPGTSVRVSCKASG | DIVLTQTPGTLSLSPGERATLSCRASQSV |
| | | FTFTSSAVQWVRQARGQRLEWVGWIV | SSSYLAWYQQKPGQAPRLLIYGASSRATG |
| | | VGSGNTNYAQKFHERVTITRDMSTST | IPDRFSGSGSGTDFTLTISRLEPEDFAVY |
| | | AYMELSSLRSEDTAVYYCASPYCSGG | YCQQYVGLTGWTFGQGTKVEIK |
| | | SCSDGFDIWGQGTMVTVSS | (SEQ ID NO: 17) |
| | | (SEQ ID NO: 90) | |
| 56 | A23- | QMQLVQSGPEVKKPGTSVKVSCKASG | DIVLTQTPGTLSLSPGERATLSCRASQSV |
| | 58.1HC/S2E1 | FTFTSSAVQWVRQARGQRLEWIGWIV | SSSYLAWYQQKPGQAPRLLIYGASSRATG |
| | 2LC | VGSGNTNYAQKFQERVTITRDMSTST | IPDRFSGSGSGTDFTLTISRLEPEDFAVY |
| | | AYMELSSLRSEDTAVYYCAAPNCSNV | YCQQYVGLTGWTFGQGTKVEIK |
| | | VCYDGFDIWGQGTMVTVSS | (SEQ ID NO: 17) |
| | | (SEQ ID NO: 10) | |
| 57 | A23- | QMQLVQSGPEVKKPGTSVKVSCKASG | DIVLTQTPGTLSLSPGERATLSCRASQSV |
| | 58.1HC_Y100 | FTFTSSAVQWVRQARGQRLEWIGWIV | SSSYLAWYQQKPGQAPRLLIYGASSRATG |
| | cA/S2E12LC | VGSGNTNYAQKFQERVTITRDMSTST | IPDRFSGSGSGTDFTLTISRLEPEDFAVY |
| | | AYMELSSLRSEDTAVYYCAAPNCSNV | YCQQYVGLTGWTFGQGTKVEIK |
| | | VCADGFDIWGQGTMVTVSS | (SEQ ID NO: 17) |
| | | (SEQ ID NO: 15) | |
| 58 | S2E12HC/A23 | QVQLVQSGPEVKKPGTSVRVSCKASG | EIVLTQSPGTLSLSPGERATLSCRASQSV |
| | -58.1LC | FTFTSSAVQWVRQARGQRLEWVGWIV | SSSYLAWYQQKPGQAPRLLIYSASSRATG |
| | | VGSGNTNYAQKFHERVTITRDMSTST | IPDRFSGSGSGTDFTLTISRLEPEDFAVY |
| | | AYMELSSLRSEDTAVYYCASPYCSGG | FCQQYGTSPWTFGQGTKVEIK |
| | | SCSDGFDIWGQGTMVTVSS | (SEQ ID NO: 70) |
| | | (SEQ ID NO: 90) | |
| 59 | B1- | QMQLVQSGPEVKKPGTSVKVSCKASG | DIVLTQTPGTLSLSPGERATLSCRASQSV |
| | 182.1HC/S2E | FTFTSSAVQWVRQARGQRLEWIGWIV | SSSYLAWYQQKPGQAPRLLIYGASSRATG |
| | 12LC | VGSGNTNYAQKFQERVTITRDMSTST | IPDRFSGSGSGTDFTLTISRLEPEDFAVY |
| | | AYMELSSLRSEDTAVYYCAAPYCSGG | YCQQYVGLTGWTFGQGTKVEIK |
| | | SCFDGFDIWGQGTMVTVSS | (SEQ ID NO: 17) |
| | | (SEQ ID NO: 1) | |
| 60 | B1- | QMQLVQSGPEVKKPGTSVKVSCKASG | DIVLTQTPGTLSLSPGERATLSCRASQSV |
| | 182.1HC_F10 | FTFTSSAVQWVRQARGQRLEWIGWIV | SSSYLAWYQQKPGQAPRLLIYGASSRATG |
| | 0cS/S2E12LC | VGSGNTNYAQKFQERVTITRDMSTST | IPDRFSGSGSGTDFTLTISRLEPEDFAVY |
| | | AYMELSSLRSEDTAVYYCAAPYCSGG | YCQQYVGLTGWTFGQGTKVEIK |
| | | SCSDGFDIWGQGTMVTVSS | (SEQ ID NO: 17) |
| | | (SEQ ID NO: 46) | |
| 61 | S2E12H/B1- | QVQLVQSGPEVKKPGTSVRVSCKASG | EIVLTQSPGTLSLSPGERATLSCRASQSV |
| | 182.1LC | FTFTSSAVQWVRQARGQRLEWVGWIV | SSSYLAWYQQKPGQAPRLLIYGASSRATG |
| | | VGSGNTNYAQKFHERVTITRDMSTST | FPDRFSGSGSGTDFTLTISRLEPEDFAVY |
| | | AYMELSSLRSEDTAVYYCASPYCSGG | YCQQYGNSPWTFGQGTKVEIR |
| | | SCSDGFDIWGQGTMVTVSS | (SEQ ID NO: 20) |
| | | (SEQ ID NO: 90) | |

### Example 2

### Stability and Aggregative Potential

It was previously determined that B1-182.1_58CDRH3 heavy/B1-182.1 (antibody 3) light showed improved yield, ability to concentrate and did not show precipitation, we designed a further 13 designs (mAbs 5-17 in Table 2 and 3) based upon antibody 3 and found 11 which did not have precipitation. Of these, 8 with yielded >150 mg/Liter (Table 3).

TABLE 3
Yield, concentration and precipitation properties for B1-182.1 variations. Yield
was determined using transient transfection (TF) of a 250 mL culture. Shown
are actual and projected 1 L yields. High-yield is defined as >125 mg/L.

| | | Final | Yield | Projected | |
|---|---|---|---|---|---|
| | | concen. | (mg/from | Yield | |
| Name | mAb # | (mg/ml) | 250 ml TF) | (mg/L) | Note |
| B1-182.1_HC_G-StoN-V | 10 | 3.3 | 57.7 | 230.9 | No precipitation |
| B1-182.1_HC_GGStoNVV | 5 | 2.6 | 54.2 | 216.7 | No precipitation |
| B1-182.1_HC_Y-StoN-V | 12 | 1.9 | 48.3 | 193.0 | No precipitation |
| B1-182.1_HC_YGGtoNNV | 14 | 2.1 | 46.0 | 183.9 | No precipitation |
| B1-182.1_HC_GStoVV | 6 | 3.7 | 44.9 | 179.5 | No precipitation |
| B1-182.1_HC_GGtoNV | 13 | 3.1 | 44.3 | 177.4 | No precipitation |
| B1-182.1_HC_Y96N | 9 | 2.0 | 36.3 | 145.1 | No precipitation |
| B1-182.1_HC_S100aV | 7 | 1.8 | 33.4 | 133.5 | No precipitation |
| B1-182.1_HC_YSFtoNVY | 11 | 2.9 | 50.4 | 201.5 | No precipitation |
| B1-182.1_HC_-GSFto-VVY | 15 | 2.3 | 38.8 | 155.3 | No precipitation |
| B1-182.1_HC_G100V | 16 | 1.8 | 30.2 | 120.6 | Turbidness observed during |
| | | | | | dialysis. Little precipitation |
| | | | | | and cloudiness observed |
| | | | | | during concentration at |
| | | | | | higher conc |
| B1-182.1_HC_G99N | 17 | 1.4 | 23.6 | 94.4 | Turbidness observed during |
| | | | | | dialysis. Little precipitation |
| | | | | | and cloudiness observed |
| | | | | | during concentration at |
| | | | | | higher conc |
| B1-182.1_HC_F100cY | 8 | 1.3 | 21.4 | 85.4 | No precipitation |

In the case of all 13 antibodies, each retained ultrapotent neutralization activity against D614G with IC50s ranging from 0.9 to 4.9 ng/ml (FIGS. 1A-1F, antibodies 5-17). Each of these antibodies was also tested against the BA.1 (Omicron) variant and showed neutralization that was between 52 and 357 ng/ml (FIGS. 1A-1F 1, antibodies 5-17). This represents high potency against Omicron and is similar to or better than what was reported antibodies used clinically against BA.1 (Zhou, et al. Science 2022).

The following neutralization characteristics were determined:
B1-182.1HC_GGStoNVV/182.1LC (Antibody 5). G99N/G100V/S100aV (Kabat) showed BA.1 neutralization IC50 of 52 ng/mL (>3.5-fold improved), high yield by transient transfection and did not precipitate.
B1-182.1HC_GStoVV/182.1LC (Antibody 6). G100V/S100aV (Kabat) showed BA.1 neutralization IC50 of 55.3 ng/ml (>3.5-fold improved), high yield by transient transfection and did not precipitate.
B1-182.1HC_S100aV/182.1LC (Antibody 7). S100aV (Kabat) showed BA.1 neutralization IC50 of 58.9 ng/mL (>3-fold improved), high yield by transient transfection and did not precipitate.
B1-182.1HC_F100cY/182.1LC (Antibody 8). F100cY (Kabat) showed BA.1 neutralization IC50 of 77.2 ng/ml (>2.5-fold improved) and did not precipitate. However, the yield was similar to parental

B1.182.1.
B1-182.1HC_Y96N/182.1LC (Antibody 9). Y96N (Kabat) showed BA. 1 neutralization IC50 of 143 ng/mL (<2-fold improved), high yield by transient transfection and did not precipitate.
B1-182.1HC_G-StoN-V/182.1LC (Antibody 10). G99N/S100aV (Kabat) showed BA.1 neutralization IC50 of 141 ng/mL (<2-fold improved), high yield by transient transfection and did not precipitate.
B1-182.1HC_YSFtoNVY/182.1LC (Antibody 11). Y96N/S100aV/F100dY (Kabat) showed BA.1 neutralization IC50 of 122 ng/mL (>2-fold improved), high yield by transient transfection and did not precipitate.
B1-182.1HC_Y-StoN-V/182.1LC (Antibody 12). Y96N/S100aV (Kabat) showed BA.1 neutralization IC50 of 174 ng/ml (minimal change), high yield by transient transfection and did not precipitate.
B1-182.1HC_GGtoNV/182.1LC (Antibody 13). G99N/G100V (Kabat) showed BA.1 neutralization IC50 of 324 ng/ml (<2-fold worse), high yield by transient transfection and did not precipitate.
B1-182.1HC_YGGtoNNV/182.ILC (Antibody 14). GY96N/99N/G100V (Kabat) showed BA.1 neutralization IC50 of 357 ng/ml (<2-fold worse), high yield by transient transfection and did not precipitate.
B1-182.1HC_-GSFto-VVY/182.1LC (Antibody 15). G100V/S100aV/F100dY (Kabat) showed BA.1 neutralization IC50 of 145 ng/mL (<2-fold improved), high yield by transient transfection and did not precipitate.
B1-182.1HC_G100V/182.1LC (Antibody 16). G100V (Kabat) showed BA.1 neutralization IC50 of 82.7 ng/mL (>2-fold improved). However, while the yield was moderately improved, precipitation was observed.
B1-182.1HC_G99N/182.1LC (Antibody 17). G99N (Kabat) showed BA.1 neutralization IC50 of 232 ng/mL (<2-fold worse), no yield improved was observed and precipitation was observed.

### Example 3

### Improving Potency and Breadth of Neutralization for Omicron and Omicron Sublineages

B1-182.1 and A23-58.1 are broadly neutralizing antibodies that are highly potent against variants that appeared prior to Omicron. However, Omicron and its sublineages have been noted to show a >10-fold decrease in potency for these antibodies and related antibodies reported in the literature. In particular, the BA.4 and BA.5 variants of Omicron have been shown to almost completely eliminate neutralization by antibodies that target the same epitope as B1-182.1 and A23-58.1 (Zhou et al Science 2022). Similar loss of neutralization function against BA.4/5 was observed for A23-58.1 and B1-182.1 (FIGS. 1A-1F). One related antibody, S2E12 (Table 3 and FIGS. 1A-1F, Antibody 55) had a <10-fold loss in neutralization against BA.1 but lost >100-fold loss in neutralization potency against BA.4/5.

Variations in B1-182.1 and A23-58.1 that would increase potency against the Omicron and the Omicron sublineage variants of SARS-CoV-2 were designed. Forty-three different variants of A23-58.1 and B1-182.1 heavy and light chains (Table 3, Antibodies 18-54, 58-61) were designed and evaluated for this reason. The following variations were produced:
1) Position 100c in the CDRH3 was explored based on structural analyses which had suggested might improve neutralization of Omicron variants.
2) Groups of amino acids or single amino acids were changed in the heavy and/or light chain to residues found in the related antibodies to explore the impact of sequence variation on neutralization and determine their impact on neutralization of Omicron and related viruses.
   Amongst the 19 variations of B1-182.1 (Antibodies 18-33, 59-61) against BA.1, 2 antibodies with IC50 values improved >20-fold (i.e., <10 ng/mL) and 12 that were improved >2-fold (i.e., 10-100 ng/mL) (FIGS. 1A-1F) were found. For the 24 A19-58.1 variants (Antibodies 34-54,56-58) against BA.1, 10 antibodies with IC50 values improved >10-fold (i.e., <10 ng/ml) and 9 improved >2-fold (i.e., 10-50 ng/ml) were found (FIGS. 1A-1F).

BA.4/5 is highly resistant against many antibodies, including antibodies related B1.182.1 and A19-58.1. B1.-182.1 and A19-58.1 are non-neutralizing against BA.4/5 viruses (FIGS. 1A-1F). In addition, S2E12 shows weak neutralization against BA.4/5 with an IC50 of 713 ng/mL compared to 1.1 ng/ml against D614G and 8.8 ng/mL against BA.1 (FIGS. 1A-1F). The loss of neutralization against BA.4/5 by antibodies in the same family is due to the amino acid substitution in BA4/5 of F486V, which has previously been shown to be a critical contact for these antibodies (Wang et al., Science 2021). Therefore, 40 of the antibodies were assayed for neutralization against BA.4/5. For the 17 variants of B1-182.1 that were investigated, 1 had an IC50 of <20 ng/mL and 2 were between 20-100 ng/ml (FIGS. 1A-1F). For the 23 variants of A19-58.1, 3 variants had an IC50 of <10 ng/mL, 1 between 10-20 ng/ml and 4 were between 20-100 ng/ml (FIGS. 1A-1F).

The following binding characteristics were determined:
B1-182.1HC_F100cS/182.1LC (Antibody 18) showed IC50 of 34.6 ng/ml (>5-fold improvement over B1-182.1) for BA.1, 14.1 ng/mL for BA.1.1, 68.9 ng/ml for BA.2, 39.5 ng/mL for BA.2.12 and did not neutralize BA.4/5.
B1-182.1HC_F100cG/182.1LC (Antibody 19) showed poor neutralization against BA.1 and did not neutralize BA.4/5.
B1-182.1HC/182.1LC_F59I (Antibody 20) showed poor neutralization against BA.1 and did not neutralize BA.4/5.
B1-182.1HC/182.1LC_G93V (Antibody 21) showed IC50 of 90.4 ng/ml (>2-fold improvement over B1-182.1) for BA.1 but did not neutralize BA.4/5.
B1-182.1HC/182.1LC_N94G (Antibody 22) showed IC50 of 62.3 ng/mL (>3-fold improvement over B1-182.1) for BA.1 but did not neutralize BA.4/5.
B1-182.1HC/182.1LC_S95L (Antibody 23) showed IC50 of 57.5 ng/mL (>3-fold improvement over B1-182.1) for BA.1 but did not neutralize BA.4/5.
B1-182.1HC/182.1LC_P96T (Antibody 24) did not neutralize BA.1 or BA.4/5.
B1-182.1HC/182.1LC_94GLT (Antibody 25) showed IC50 of 17.7 ng/ml (>11-fold improvement over B1-182.1) for BA.1 but did not neutralize BA.4/5.
B1-182.1HC/182.1LC_93VGLT (Antibody 26) did not improve BA.1 neutralization over B1-182.1 and did not neutralize BA.4/5.
B1-182.1HC/182.1LC_94GLTG (Antibody 27) showed ultra-potent neutralization IC50 between 1.6-14.2 ng/mL for D614G, B.1.617.2, B.1.351 and BA.1. The improvement for BA.1 was >13-fold over B1-182.1. This antibody also potently neutralized BA.4/5 at 14.1 ng/ml under conditions where the parental antibody did not neutralize. Its potency is 50-fold better than S2E12.
B1-182.1HC/182.1LC_93VGLTG (Antibody 28) showed ultra-potent neutralization IC50 between 1.4-11.5 ng/ml for D614G, B.1.617.2, B.1.351 and BA.1. The improvement for BA.1 was >17-fold over B1-182.1. This antibody also potently neutralized BA.4/5 at 28.8 ng/mL under conditions where the parental antibody did not neutralize. Its potency is 25-fold better than S2E12.
B1-182.1HC/182.1LC_95LTG (Antibody 29) showed ultra-potent neutralization IC50 between 3.9-20.6 ng/mL for D614G, B.1.617.2, B.1.351 and BA.1. The improvement for BA.1 was >9-fold over B1-182.1. This antibody weakly neutralized BA.4/5.
B1-182.1HC/182.1LC_96TG (Antibody 30) showed ultra-potent neutralization IC50 between 3.8-20.6 ng/ml for D614G, B.1.617.2, B.1.351 and BA.1. The improvement for BA.1 was >9-fold over B1-182.1. This antibody did not neutralize BA.4/5.
B1-182.1HC/182.1LC_96+G (Antibody 31) showed ultra-potent neutralization against D614G, B.1.617.2 and B.1.351, but did not improve BA.1 neutralization over B1-182.1 and did not neutralize BA.4/5
B1-182.1HC_F100cS/182.1LC_94GLTG (Antibody 32) showed ultra-potent neutralization IC50 between 1.8-4.2 ng/mL for D614G, B.1.617.2, B.1.351 and BA.1. The improvement for BA.1 was >46-fold over B1-182.1. This antibody weakly neutralized BA.4/5.
B1-182.1HC_F100cS/182.1LC_93VGLTG (Antibody 33) showed ultra-potent neutralization of BA.1 with an IC50 of 22.8 ng/ml (>9-fold improved over B1-182.1). This antibody did not neutralize BA.4/5.
A23-58.1HC_Y100cS/58.1LC (Antibody 34) weakly neutralized BA.1.
A23-58.1HC_Y100cA/58.1LC (Antibody 35) showed ultra-potent neutralization IC50 between 17.3-27.8 ng/mL for BA.1, BA.1.1, BA.2 and BA.2.12.1. The improvement for BA.1 was >6-fold over A19-58.1. This antibody did not neutralize BA.4/5.
A23-58.1HC/58.1LC_S51G (Antibody 36) showed highly potent neutralization IC50 of 28.9 ng/ml for BA.1 (3-fold improvement over A19-58.1) and 50.2 ng/mL for BA.2.12.1. This antibody did not neutralize BA.4/5.
A23-58.1HC/58.1LC_F88Y (Antibody 37) showed ultra-potent neutralization IC50 of 3.4 ng/ml for BA.1 (29-fold improvement over A19-58.1) and 10.1 ng/mL for BA.2.12.1. This antibody did not neutralize BA.4/5.
A23-58.1HC/58.1LC_G93V (Antibody 38) showed ultra-potent neutralization IC50 of 5.5 ng/ml for BA.1 (18-fold improvement over A19-58.1) and high potency neutralization at 31.6 ng/ml for BA.2.12.1. This antibody did not neutralize BA.4/5.
A23-58.1HC/58.1LC_T94G (Antibody 39) showed ultra-potent neutralization IC50 of 14.8 ng/ml for BA.1 (7-fold improvement over A19-58.1) and 13.7 ng/ml for BA.2.12.1. This antibody did not neutralize BA.4/5.
A23-58.1HC/58.1LC_S95L (Antibody 40) showed ultra-potent neutralization IC50 of 6.1 ng/ml for BA.1 (16-fold improvement over A19-58.1) and 7.2 ng/mL for BA.2.12.1. This antibody did not neutralize BA.4/5.
A23-58.1HC/58.1LC_P96T (Antibody 41) showed similar neutralization for to A19-58.1 for BA.1 and BA.2.12.1. It did not neutralize BA.4/5.
A23-58.1HC/58.1LC_94GLT (Antibody 42) showed ultra-potent neutralization IC50 of 13.7 ng/mL for BA.1 (7-fold improvement over A19-58.1) and 25 ng/mL for BA.2.12.1. This antibody did not neutralize BA.4/5.
A23-58.1HC/58.1LC_93VGLT (Antibody 43) showed ultra-potent neutralization IC50 between 0.9-4.8 ng/mL for D614G, B.1.617.2, B.1.351 and BA.1. The improvement for BA.1 was >20-fold over A19-58.1. This antibody did not neutralize BA.4/5.
A23-58.1HC/58.1LC_94GLTG (Antibody 44) showed ultra-potent neutralization IC50 between 1.8-3.6 ng/mL for D614G, B.1.617.2, B.1.351, BA.1 and BA.2.12.1. The improvement for BA.1 was >27-fold over A19-58.1. This antibody was ultra-potent against BA.4/5 with an IC50 of 4.7 ng/ml and is >150-fold better than S2E12.
A23-58.1HC/58.1LC_93VGLTG (Antibody 45) showed ultra-potent neutralization IC50 between 2.7-5.8 ng/mL for D614G, B.1.617.2, B.1.351, BA.1 and BA.2.12.1. The improvement for BA.1 was >30-fold over A19-58.1. This antibody was ultra-potent against BA.4/5 with an IC50 of 4.7 ng/ml and is >235-fold better than S2E12.
A23-58.1HC/58.1LC_95LTG (Antibody 46) showed ultra-potent neutralization IC50 between 1.4-21.4 ng/ml for D614G, B.1.617.2, B.1.351 and BA.1. The improvement for BA.1 was 5-fold over A19-58.1. This antibody weakly neutralized BA.4/5.
A23-58.1HC/58.1LC_96TG (Antibody 47) showed high to ultra-potent neutralization IC50 between 1.3-29.4 ng/mL for D614G, B.1.617.2, B.1.351 and BA.1. The improvement for BA.1 was >3-fold over A19-58.1. This antibody did not neutralize BA.4/5.
A23-58.1HC/58.1LC_96+G (Antibody 48) showed no improvement over A19-58.1 for D614G, B.1.617.2, B.1.351 and BA.1. This antibody did not neutralize BA.4/5.
A23-58.1HC/58.1LC_F88Y-94GLTG (Antibody 49) showed high to ultra-potent neutralization IC50 between 2.3-41.7 ng/mL for D614G, B.1.617.2, B.1.351, BA.1 and BA.2.12.1. The improvement for BA.1 was >3-fold over A19-58.1. This antibody was moderately potent against BA.4/5 with an IC50 of 164 ng/ml and is >4-fold better than S2E12.
A23-58.1HC/58.1LC_F88Y-93VGLTG (Antibody 50) showed ultra-potent neutralization IC50 between 4.7-9.9 ng/mL for D614G, B.1.617.2, B.1.351, BA.1 and BA.2.12.1. The improvement for BA.1 was >10-fold over A19-58.1. This antibody was highly-potent against BA.4/5 with an IC50 of 38.8 ng/mL and is >18-fold better than S2E12.
A23-58.1HC_Y100cA/58.1LC_S95L (Antibody 51) showed ultra-potent neutralization IC50 of 19.4 ng/ml for BA.1 (5-fold improvement over A19-58.1) and 29.6 ng/ml for BA.2.12.1. This antibody did not neutralize BA.4/5.
A23-58.1HC_Y100cA/58.1LC_93VGLT (Antibody 52) did not show improved neutralization for BA.1 and BA.2.12.1. It did not neutralized BA.4/5.
A23-58.1HC_Y100cA/58.1LC_94GLTG (Antibody 53) showed high to ultra-potent neutralization IC50 between 2.3-19.3 ng/mL for D614G, B.1.617.2, B.1.351, BA.1 and BA.2.12.1. The improvement for BA.1 was >25-fold over A19-58.1. This antibody was moderately potent against BA.4/5 with an IC50 of 144 ng/ml and is 5-fold better than S2E12.
A23-58.1HC_Y100cA/58.1LC_93VGLTG (Antibody 54) showed ultra-potent neutralization IC50 between 1.4-24.2 ng/mL for D614G, B.1.617.2, B.1.351, BA.1 and BA.2.12.1. The improvement for BA.1 was >6-fold over A19-58.1. This antibody was highly-potent against BA.4/5 with an IC50 of 21 ng/ml and is 34-fold better than S2E12.
A23-58.1HC/S2E12LC (Antibody 56) showed high to ultra-potent neutralization IC50 between 2.2-56.6 ng/ml for D614G, B.1.617.2, B.1.351, BA.1, BA.1.1, BA.2 and BA.2.12.1. The improvement for BA.1 was 10-fold over A19-58.1. This antibody was ultra-potent against BA.4/5 with an IC50 of 9.9 ng/ml and is >70-fold better than S2E12.
A23-58.1HC_Y100cA/S2E12LC (Antibody 57) showed high to ultra-potent neutralization IC50 between 1.4-35.1 ng/mL for D614G, B.1.617.2, B.1.351, BA.1, BA.1.1, BA.2 and BA.2.12.1. The improvement for BA.1 was 20-fold over A19-58.1. This antibody was ultra-potent against BA.4/5 with an IC50 of 13.5 ng/ml and is >50-fold better than S2E12.
S2E12HC/A23-58.1LC (Antibody 58). This antibody showed worse BA.1 neutralization than A19-58.1 and S2E12 and did not neutralize BA.4/5
B1-182.1HC/S2E12LC (Antibody 59) showed ultra-potent neutralization IC50 between 0.8-11.5 ng/mL for D614G, B.1.617.2, B.1.351, BA.1, BA.1.1, BA.2 and BA.2.12.1. The improvement for BA.1 was 20-fold over B1-182.1. This antibody was highly-potent against BA.4/5 with an IC50 of 49.7 ng/ml and is >14-fold better than S2E12.
B1-182.1HC_F100cS/S2E12LC (Antibody 60) showed ultra-potent neutralization IC50 between 1.2-14.9 ng/ml for D614G, B.1.617.2, B.1.351, BA.1, BA.1.1, BA.2 and BA.2.12.1. The improvement for BA.1 was >14-fold over B1-182.1. This antibody did not neutralize BA.4/5.
S2E12H/B1-182.1LC (Antibody 61) did not improve neutralization of BA.1 and did not neutralize BA.4/5

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An isolated monoclonal antibody or antigen binding fragment thereof, comprising a heavy chain variable (V_{H}) region and a light chain variable region (V_{L}) comprising a heavy chain complementarity determining region (HCDR)1, a HCDR2, and a HCDR3, and a light chain complementarity determining region (LCDR)1, a LCDR2, and a LCDR3 of the V_{H} and V_{L} set forth as any one of:
a) SEQ ID NOs: 1 and 5, respectively (B1-182.1HC/182.1LC_94GLTG);
b) SEQ ID NOs: 1 and 8, respectively (B1-182.1HC/182.1LC_93VGLTG);
c) SEQ ID NOs: 10 and 12, respectively (A23-58.1HC/58.1LC_94GLTG);
d) SEQ ID NOs: 10 and 13, respectively (A23-58.1HC/58.1LC_93VGLTG);
e) SEQ ID NOs: 10 and 14, respectively (A23-58.1HC/58.ILC_F88Y-93VGLTG);
f) SEQ ID NOs: 15 and 13, respectively (A23-58.1HC_Y100cA/58.1LC_93VGLTG);
g) SEQ ID NOs: 10 and 17, respectively (A23-58.1HC/S2E12LC);
h) SEQ ID NOs: 15 and 17, respectively (A23-58.1HC_Y100cA/S2E12LC);
i) SEQ ID NOs: 1 and 17, respectively (B1-182.1HC/S2E12LC);
j) SEQ ID NOs: 18 and 20, respectively (B1-182.1HC_GGStoNVV/182.1LC);
k) SEQ ID NOs: 22 and 20, respectively (B1-182.1HC_GStoVV/182.1LC);
l) SEQ ID NOs: 24 and 20, respectively (B1-182.1HC_S100aV/182.1LC);
m) SEQ ID NOs: 26 and 20, respectively (B1-182.1HC_F100cY/182.1LC);
n) SEQ ID NOs: 28 and 20, respectively (B1-182.1HC_Y96N/182.1LC);
o) SEQ ID NOs: 30 and 20, respectively (B1-182.1HC_G-StoN-V/182.1LC);
p) SEQ ID NOs: 32 and 20, respectively (B1-182.1HC_YSFtoNVY/182.1LC);
q) SEQ ID NOs: 34 and 20, respectively (B1-182.1HC_Y-StoN-V/182.ILC);
r) SEQ ID NOs: 36 and 20, respectively (B1-182.1HC_GGtoNV/182.ILC);
s) SEQ ID NOs: 38 and 20, respectively (B1-182.1HC_YGGtoNNV/182.1LC);
t) SEQ ID NOs: 40 and 20, respectively (B1-182.1HC_-GSFto-VVY/182.1LC);
u) SEQ ID NOs: 42 and 20, respectively (B1-182.1HC_G100V/182.1LC);
v) SEQ ID NOs: 44 and 20, respectively (B1-182.1HC_G99N/182.1LC);
w) SEQ ID NOs: 46 and 20, respectively (B1-182.1HC_F100cS/182.ILC);
x) SEQ ID NOs: 1 and 50, respectively (B1-182.1HC/182.1LC_G93V);
y) SEQ ID NOs: 1 and 52, respectively (B1-182.1HC/182.1LC_N94G);
z) SEQ ID NOs: 1 and 54, respectively (B1-182.1HC/182.1LC_S95L);
aa) SEQ ID NOs: 1 and 58, respectively (B1-182.1HC/182.1LC_94GLT);
bb) SEQ ID NOs: 1 and 62, respectively (B1-182.1HC/182.1LC_95LTG);
cc) SEQ ID NOs: 1 and 64, respectively (B1-182.1HC/182.1LC_96TG);
dd) SEQ ID NOs: 1 and 66, respectively (B1-182.1HC/182.1LC_96+G);
ee) SEQ ID NOs: 46 and 5, respectively (B1-182.1HC_F100S/182.1LC_94GLTG);
ff) SEQ ID NOs: 46 and 8, respectively (B1-182.1HC_F100cS/182.1LC_93VGLTG);
gg) SEQ ID NOs: 68 and 70, respectively (A23-58.1HC_Y100cS/58.ILC);
hh) SEQ ID NOs: 15 and 70, respectively (A23-58.1HC_Y100cA/58.ILC);
ii) SEQ ID NOs: 10 and 72, respectively (A23-58.1HC/58.1LC_S51G);
jj) SEQ ID NOs: 10 and 73, respectively (A23-58.1HC/58.1LC_F88Y);
kk) SEQ ID NOs: 10 and 74, respectively (A23-58.1HC/58.1LC_G93V);
ll) SEQ ID NOs: 10 and 76, respectively (A23-58.1HC/58.1LC_T94G);
mm) SEQ ID NOs: 10 and 77, respectively (A23-58.1HC/58.1LC_S95L);
nn) SEQ ID NOs: 10 and 79, respectively (A23-58.1HC/58.1LC_P96T);
oo) SEQ ID NOs: 10 and 81, respectively (A23-58.1HC/58.1LC_94GLT);
pp) SEQ ID NOs: 10 and 82, respectively (A23-58.1HC/58.1LC_93VGLT);
qq) SEQ ID NOs: 10 and 83, respectively (A23-58.1HC/58.1LC_95LTG);
rr) SEQ ID NOs: 10 and 85, respectively (A23-58.1HC/58.ILC_96TG);
ss) SEQ ID NOs: 10 and 89, respectively (A23-58.1HC/58.1LC_F88Y-94GLTG);
tt) SEQ ID NOs: 15 and 77, respectively (A23-58.1HC_Y100cA/58.1LC_S95L);
uu) SEQ ID NOs: 15 and 12, respectively (A23-58.1HC_Y100cA/58.1LC_94GLTG); or
vv) SEQ ID NOs: 46 and 17, respectively (B1-182.1HC_F100cS/S2E12LC);
and wherein the monoclonal antibody specifically binds to a severe acute respiratory syndrome coronavirus 2 (SARS-CoV-2) spike protein.

2. The isolated monoclonal antibody or antigen binding fragment of claim 1, wherein the HCDR1, the HCDR2, the HCDR3, the LCDR1, the LCDR2, and the LCDR3 comprise the amino acids sequences set forth as:
a) SEQ ID NOs: 2, 3, 4, 6, GAS, and 7, respectively;
b) SEQ ID NOs: 2, 3, 4, 6, GAS, and 9, respectively;
c) SEQ ID NOs: 2, 3, 11, 6, SAS, and 7, respectively;
d) SEQ ID NOs: 2, 3, 11, 6, SAS, and 9, respectively;
e) SEQ ID NOs: 2, 3, 16, 6, SAS, and 9, respectively;
f) SEQ ID NOs: 2, 3, 11, 6, GAS, and 9, respectively;
g) SEQ ID NOs: 2, 3, 16, 6, GAS, and 9, respectively;
h) SEQ ID NOs: 2, 3, 4, 6, GAS, and 9, respectively;
i) SEQ ID NOs: 2, 3, 19, 6, GAS, and 21, respectively;
j) SEQ ID NOs: 2, 3, 23, 6, GAS, and 21, respectively;
k) SEQ ID NOs: 2, 3, 25, 6, GAS, and 21, respectively;
l) SEQ ID NOs: 2, 3, 27, 6, GAS, and 21, respectively;
m) SEQ ID NOs: 2, 3, 29, 6, GAS, and 21, respectively;
n) SEQ ID NOs: 2, 3, 31, 6, GAS, and 21, respectively;
o) SEQ ID NOs: 2, 3, 33, 6, GAS, and 21, respectively;
p) SEQ ID NOs: 2, 3, 35, 6, GAS, and 21, respectively;
q) SEQ ID NOs: 2, 3, 37, 6, GAS, and 21, respectively;
r) SEQ ID NOs: 2, 91, 39, 6, GAS, and 21, respectively;
s) SEQ ID NOs: 2, 3, 41, 6, GAS, and 21, respectively;
t) SEQ ID NOs: 2, 3, 43, 6, GAS, and 21, respectively;
u) SEQ ID NOs: 2, 3, 45, 6, GAS, and 21, respectively;
v) SEQ ID NOs: 2, 3, 47, 6, GAS, and 21, respectively;
w) SEQ ID NOs: 2, 3, 4, 6, GAS, and 51, respectively;
x) SEQ ID NOs: 2, 3, 4, 6, GAS, and 53, respectively;
y) SEQ ID NOs: 2, 3, 4, 6, GAS, and 55, respectively;
z) SEQ ID NOs: 2, 3, 4, 6, GAS, and 59, respectively;
aa) SEQ ID NOs: 2, 3, 4, 6, GAS, and 63, respectively;
bb) SEQ ID NOs: 2, 3, 4, 6, GAS, and 65, respectively;
cc) SEQ ID NOs: 2, 3, 4, 6, GAS, and 67, respectively;
dd) SEQ ID NOs: 2, 3, 47, 6, GAS, and 7, respectively;
ee) SEQ ID NOs: 2, 3, 47, 6, GAS, and 9, respectively;
ff) SEQ ID NOs: 2, 3, 69, 6, SAS, and 71, respectively;
gg) SEQ ID NOs: 2, 3, 16, 6, SAS, and 71, respectively;
hh) SEQ ID NOs: 2, 3, 11, 6, GAS, and 71, respectively;
ii) SEQ ID NOs: 2, 3, 11, 6, SAS, and 71, respectively;
jj) SEQ ID NOs: 2, 3, 11, 6, SAS, and 75, respectively;
kk) SEQ ID NOs: 2, 3, 11, 6, SAS, and 53, respectively;
ll) SEQ ID NOs: 2, 3, 11, 6, SAS, and 78, respectively;
mm) SEQ ID NOs: 2, 3, 11, 6, SAS, and 80, respectively;
nn) SEQ ID NOs: 2, 3, 11, 6, SAS, and 59, respectively;
oo) SEQ ID NOs: 2, 3, 11, 6, SAS, and 61, respectively;
pp) SEQ ID NOs: 2, 3, 11, 6, SAS, and 84, respectively;
qq) SEQ ID NOs: 2, 3, 11, 6, SAS, and 86, respectively;
rr) SEQ ID NOs: 2, 3, 11, 6, SAS, 7, respectively;
ss) SEQ ID NOs: 2, 3, 16, 6, SAS, and 78, respectively;
tt) SEQ ID NOs: 2, 3, 16, 6, SAS, and 7, respectively; or
uu) SEQ ID NOs: 2, 91, 47, 6, GAS, and 9, respectively.

3. The isolated monoclonal antibody or antigen binding fragment of claim 1, wherein the V_{H} and the V_{L} comprise the amino acid sequences at least 90% identical to the amino acid sequences set forth as:
a) SEQ ID NOs: 1 and 5, respectively;
b) SEQ ID NOs: 1 and 9, respectively;
c) SEQ ID NOs: 10 and 12, respectively;
d) SEQ ID NOs: 10 and 13, respectively;
e) SEQ ID NOs: 10 and 14, respectively;
f) SEQ ID NOs: 15 and 13, respectively;
g) SEQ ID NOs: 10 and 17, respectively;
h) SEQ ID NOs: 15 and 17, respectively;
i) SEQ ID NOs: 1 and 17, respectively;
j) SEQ ID NOs: 18 and 20, respectively;
k) SEQ ID NOs: 22 and 20, respectively;
l) SEQ ID NOs: 24 and 20, respectively;
m) SEQ ID NOs: 26 and 20, respectively;
n) SEQ ID NOs: 28 and 20, respectively;
o) SEQ ID NOs: 30 and 20, respectively;
p) SEQ ID NOs: 32 and 20, respectively;
q) SEQ ID NOs: 34 and 20, respectively;
r) SEQ ID NOs: 36 and 20, respectively;
s) SEQ ID NOs: 38 and 20, respectively;
t) SEQ ID NOs: 40 and 20, respectively;
u) SEQ ID NOs: 42 and 20, respectively;
v) SEQ ID NOs: 44 and 20, respectively;
w) SEQ ID NOs: 46 and 20, respectively;
x) SEQ ID NOs: 1 and 50, respectively;
y) SEQ ID NOs: 1 and 52, respectively;
z) SEQ ID NOs: 1 and 54, respectively;
aa) SEQ ID NOs: 1 and 58, respectively;
bb) SEQ ID NOs: 1 and 62, respectively;
cc) SEQ ID NOs: 1 and 64, respectively;
dd) SEQ ID NOs: 1 and 66, respectively;
ee) SEQ ID NOs: 46 and 5, respectively;
ff) SEQ ID NOs: 46 and 8, respectively;
gg) SEQ ID NOs: 68 and 70, respectively;
hh) SEQ ID NOs: 15 and 70, respectively;
ii) SEQ ID NOs: 10 and 72, respectively;
jj) SEQ ID NOs: 10 and 73, respectively;
kk) SEQ ID NOs: 10 and 74, respectively;
ll) SEQ ID NOs: 10 and 76, respectively;
mm) SEQ ID NOs: 10 and 77, respectively;
nn) SEQ ID NOs: 10 and 79, respectively;
oo) SEQ ID NOs: 10 and 81, respectively;
pp) SEQ ID NOs: 10 and 82, respectively;
qq) SEQ ID NOs: 10 and 83, respectively;
rr) SEQ ID NOs: 10 and 85, respectively;
ss) SEQ ID NOs: 10 and 89, respectively;
tt) SEQ ID NOs: 15 and 77, respectively;
uu) SEQ ID NOs: 15 and 12, respectively; or
vv) SEQ ID NOs: 46 and 17, respectively.

4. The isolated monoclonal antibody or antigen binding fragment of claim 1, wherein the V_{L} comprises SEQ ID NO: 17 or SEQ ID NO: 20.

5. The isolated monoclonal antibody or antigen binding fragment of claim 1, wherein the VA comprises SEQ ID NO: 1, SEQ ID NO: 10 or SEQ ID NO: 15.

6. The isolated monoclonal antibody or antigen binding fragment of claim 1, comprising a human framework region.

7. The isolated monoclonal antibody or antigen binding fragment of claim 1, wherein the V_{H} and the V_{L} comprise the amino acid sequences set forth as:
a) SEQ ID NOs: 1 and 5, respectively;
b) SEQ ID NOs: 1 and 8, respectively;
c) SEQ ID NOs: 10 and 12, respectively;
d) SEQ ID NOs: 10 and 13, respectively;
e) SEQ ID NOs: 10 and 14, respectively;
f) SEQ ID NOs: 15 and 13, respectively;
g) SEQ ID NOs: 10 and 17, respectively;
h) SEQ ID NOs: 15 and 17, respectively;
i) SEQ ID NOs: 1 and 17, respectively;
j) SEQ ID NOs: 18 and 20, respectively;
k) SEQ ID NOs: 22 and 20, respectively;
l) SEQ ID NOs: 24 and 20, respectively;
m) SEQ ID NOs: 26 and 20, respectively;
n) SEQ ID NOs: 28 and 20, respectively;
o) SEQ ID NOs: 30 and 20, respectively;
p) SEQ ID NOs: 32 and 20, respectively;
q) SEQ ID NOs: 34 and 20, respectively;
r) SEQ ID NOs: 36 and 20, respectively;
s) SEQ ID NOs: 38 and 20, respectively;
t) SEQ ID NOs: 40 and 20, respectively;
u) SEQ ID NOs: 42 and 20, respectively;
v) SEQ ID NOs: 44 and 20, respectively;
w) SEQ ID NOs: 46 and 20, respectively;
x) SEQ ID NOs: 1 and 50, respectively;
y) SEQ ID NOs: 1 and 52, respectively;
z) SEQ ID NOs: 1 and 54, respectively;
aa) SEQ ID NOs: 1 and 58, respectively;
bb) SEQ ID NOs: 1 and 62, respectively;
cc) SEQ ID NOs: 1 and 64, respectively;
dd) SEQ ID NOs: 1 and 66, respectively;
ee) SEQ ID NOs: 46 and 5, respectively;
ff) SEQ ID NOs: 46 and 8, respectively;
gg) SEQ ID NOs: 68 and 70, respectively;
hh) SEQ ID NOs: 15 and 70, respectively;
ii) SEQ ID NOs: 10 and 72, respectively;
jj) SEQ ID NOs: 10 and 73, respectively;
kk) SEQ ID NOs: 10 and 74, respectively;
ll) SEQ ID NOs: 10 and 76, respectively;
mm) SEQ ID NOs: 10 and 77, respectively;
nn) SEQ ID NOs: 10 and 79, respectively;
oo) SEQ ID NOs: 10 and 81, respectively;
pp) SEQ ID NOs: 10 and 82, respectively;
qq) SEQ ID NOs: 10 and 83, respectively;
rr) SEQ ID NOs: 10 and 85, respectively;
ss) SEQ ID NOs: 10 and 89, respectively;
tt) SEQ ID NOs: 15 and 77, respectively;
uu) SEQ ID NOs: 15 and 12, respectively; or
vv) SEQ ID NOs: 46 and 17, respectively.

8. The isolated monoclonal antibody of claim 1, wherein the antibody comprises a human constant domain.

9. The isolated monoclonal antibody of claim 1, wherein the antibody is a human antibody.

10. The isolated monoclonal antibody of claim 1, wherein the antibody is an IgG.

11. The isolated monoclonal antibody of claim 1, comprising a recombinant constant domain comprising a modification that increases the half-life of the antibody.

12. The isolated monoclonal antibody of claim 9, wherein the modification increases binding to the neonatal Fc receptor.

13. The antibody or antigen binding fragment of claim 1, wherein the antibody neutralizes a SARS-CoV-2 BA.4 variant or BA.5 variant.

14. The antigen binding fragment of claim 1.

15. The antigen binding fragment of claim 14, wherein the antigen binding fragment is a Fv, Fab, F(ab′)₂, scFV or a scFV₂ fragment.

16. The monoclonal antibody or antigen binding fragment of claim 1, conjugated to a detectable marker.

17. A bispecific antibody comprising the monoclonal antibody or antigen binding fragment of claim 1.

18. The bispecific antibody of claim 17, wherein the bispecific antibody is a dual variable domain immunoglobulin.

19. An isolated nucleic acid molecule encoding the monoclonal antibody or antigen binding fragment of any one of claim 1 the V_{H} or V_{L} of the antibody, the antigen binding fragment, or a dual variable domain immunoglobulin comprising the antigen binding fragment.

21. The isolated nucleic acid molecule of claim 19, wherein the nucleic acid molecule is a cDNA sequence encoding the V_{H} or V_{L}.

21. The nucleic acid molecule of claim 19, operably linked to a promoter.

22. A vector comprising the nucleic acid molecule of claim 19.

23. A host cell comprising the nucleic acid molecule or vector of claim 19.

24. A pharmaceutical composition for use in inhibiting a coronavirus infection, comprising an effective amount of the isolated monoclonal antibody or antigen binding fragment of claim 1; a bispecific antibody comprising the monoclonal antibody or antigen binding fragment; a nucleic acid molecule encoding the monoclonal antibody, the antigen binding fragment, of the bispecific antibody; or a vector comprising the nucleic acid molecule; and
a pharmaceutically acceptable carrier.

25. A method of producing a monoclonal antibody or antigen binding fragment that specifically binds to a coronavirus spike protein, comprising:
expressing one or more nucleic acid molecules encoding the monoclonal antibody or antigen binding fragment of claim 1 in a host cell; and
purifying the antibody or antigen binding fragment.

26. A method of detecting the presence of a coronavirus in a biological sample from a subject, comprising:
contacting the biological sample with an effective amount of the monoclonal antibody or antigen binding fragment of claim 1 under conditions sufficient to form an immune complex; and
detecting the presence of the immune complex in the biological sample, wherein the presence of the immune complex in the biological sample indicates the presence of the coronavirus in the sample.

27. The method of claim 26, wherein detecting the presence of the immune complex in the biological sample indicates that the subject has a SARS-CoV-2 infection.

28. A method of inhibiting a coronavirus infection in a subject, comprising administering an effective amount of the monoclonal antibody or the antigen binding fragment of claim 1, a nucleic acid molecule coding the monoclonal antibody or the antigen binding fragment, a vector comprising the nucleic acid molecule, or a pharmaceutical composition comprising the monoclonal antibody, the antigen binding fragment, the nucleic acid molecule, or the vector to the subject, wherein the subject has or is at risk of a coronavirus infection.

29. The method of claim 28, wherein the coronavirus is SARS-CoV-2.

30. The method of claim 29, wherein the SARS-CoV-2 is the BA.4 or BA.5 variant.

31. (canceled)

32. (canceled)
